(19) United States
(12) Patent Application Publication
(10) Pub. No.: US 2003/0199071 A1
(43) Pub. Date: Oct. 23, 2003

(54) Mutant proteins, high potency inhibitory antibodies and fimch crystal structure

(76) Inventors: Solomon Langermann, Baltimore, MD (US); Scott J. Hultgren, Town and Country, MO (US); Chia-Suei Hung, St. Louis, MO (US); Julie Bouckaert, St. Louis, MO (US)

Correspondence Address: PENNIE AND EDMONDS, 1155 AVENUE OF THE AMERICAS, NEW YORK, NY, 100362711

(21) Appl. No.: 10/015,085

(22) Filed: Dec. 10, 2001

Related U.S. Application Data
(60) Provisional application No. 60/254,353, filed on Dec. 8, 2000.
(60) Provisional application No. 60/301,878, filed on Jun. 29, 2001.

(51) Int. Cl. C12N 9/24; G06F 19/00; G01N 33/48; G01N 33/50

(52) U.S. Cl. 435/200; 702/19

(57) ABSTRACT

The present invention provides bacterial immunogenic agents for administration to humans and non-human animals to stimulate an immune response. It particularly relates to the vaccination of mammalian species, especially human patients, with variants of the E. coli FimCH protein that elicit antibodies that have better functional inhibitory activity than antibodies raised against wild type protein. In particular, such variants include mutations that promote a more open confirmation of the FimH protein, particularly in regions involved in mannose binding, to expose regions previously poorly exposed and mutations that abolish a significantly reduce mannose binding. In another aspect, the invention provides antibodies against such proteins and protein complexes that may be used in passive immunization to protect or treat pathogenic bacterial infections. The present invention also provides machine readable media embedded with the three-dimensional atomic structure coordinates of FimCH bound to mannose, and subsets thereof, and methods of using the crystal structure to provide candidate amino acid residues for mutation.

This application claims priority to U.S. Provisional Patent Application No. 60/254,353, filed Dec. 8, 2000, and U.S. Provisional Patent Application No. 60/301,878, filed Jun. 29, 2001, the content of each of which is incorporated herein by reference in its entirety.

### 1. FIELD OF THE INVENTION

The invention relates to methods of producing antibodies, preferably antibodies that inhibit binding of a protein to its binding partner. Further, the methods include producing antibodies having enhanced functional inhibitory activity against a protein, for example, that inhibit binding of the protein to a binding partner, by immunizing with a mutant form of the protein that elicits antibodies with greater inhibitory activity than those antibodies elicited by the wild type protein. In one example, mutant proteins are designed using the crystal structure of purified FimCH bound to mannose. Mutant proteins are expressed and used as antigens to elicit antibodies. Thus, this crystal structure, including its coordinates, and methods of designing vaccines and antibodies using information from the crystal structure are included herein. In particular embodiments, this invention relates to mutant bacterial adhesin proteins and active fragments thereof for use in the prevention, diagnosis and treatment of bacterial induced diseases such as those of the urinary tract. The invention encompasses use of mutant proteins as immunogenic agents in vaccine compositions to stimulate an immune response in humans and animals. The invention also encompasses the administration of antibodies to said mutant proteins to humans and animals in an effective amount, to treat, prevent or manage disease or infection. More specifically, the invention relates to the administration of purified mutant adhesin proteins or antibodies directed against said mutant adhesin proteins to a mammalian species as a mechanism to protect the vaccine or antibody recipient against infection by pathogenic bacterial species, including all types of Enterobacteriaceae.

### 2. BACKGROUND OF THE INVENTION

Urinary tract infections (herein, “UTI”) present a disease process that is mediated (or assisted or otherwise induced) by the attachment of bacteria to cells. Escherichia coli is the most common pathogen of the urinary tract, accounting for more than 85% of cases of asymptomatic bacteriuria, acute cystitis and acute pyelonephritis, as well as greater than 60% of recurrent cystitis, and at least 35% of recurrent pyelonephritis infections. Furthermore, approximately 25%-30% of women experience a recurrent E. coli urinary tract infection within the first 12 months following an initial infection but after a second or third infection the rate of recurrence increases to 60%-75%. Given the high incidence, continued persistence, and significant expense associated with E. coli UTI, there is a need for a prophylactic treatment to reduce susceptibility to this disease.

Despite the overall prevalence of UTI in women, there have been few efforts to apply novel strategies in order to treat and/or prevent these diseases. Commonly, conventional antibiotics are used to treat these infections, such as treatment with penicillins, cephalosporins, aminoglycosides, sulfonamides and tetracyclines; in the special case of UTI, urinary antiseptics such as nitrofarantoin and nalidixic acid are employed, too. However, emerging antibiotic resistance will in the future hamper the ability to successfully treat UTI. Multiple antibiotic resistance among these uropathogens is increasing.

While many factors contribute to the acquisition and progression of E. coli UTI, it is generally accepted that colonization of the urinary epithelium is a required step in the infection process. In a typical course of E. coli urinary tract infection, bacteria originate from the bowel, ascend into the bladder, and adhere to the bladder mucosa where they multiply and establish an infection (cystitis) before ascending into the ureters and kidneys. Disruption or prevention of pilus-mediated attachment of E. coil to urinary epithelia may prevent or retard the development of UTI. In this regard, a number of studies have pointed to a role for pili in mediating attachment to host uroepithelial cells.

The initiation and persistence of many bacterial infections such as those described above is thought to require the presentation of adhesins on the surface of the microbe in accessible configurations which promote binding events that dictate whether extracellular colonization, internalization or other cellular responses will occur. Adhesins are often components of the long, thin, filamentous, heteropolymeric protein appendages known as pili, fimbriae, or fibrillae (these three terms will be used interchangeably herein). The bacterial attachment event is often the result of a stereo-chemical fit between an adhesin frequently located at the pilus tip and specific receptor architectures on host cells, often comprising carbohydrate structures in membrane associated glycoconjugates.

Uropathogenic strains of E. coli express P and type 1 pili that bind to receptors present in uroepithelial cells. The adhesin present at the tip of the P pilus, PapG, binds to the Galα(1-4)Gal moiety present in the globoseries of glycolipids. Alternatively, the type 1 adhesin, FimH, binds D-mannose present in glycolipids and glycoproteins. Type 1 pili are thought to be important in initiating colonization of the bladder and inducing cystitis, whereas P pili are thought to play a role in ascending infections and the ensuing pyelonephritis.

With regard to type 1 pili, tip adhesins and other ancillary subunits also have been identified. FimH is the D-mannose-binding adhesin that promotes attachment of type 1 piliated bacteria to host cells via mannose-containing glycoproteins or eukaryotic cell surfaces. FimC is its periplasmic chaperone protein. It has recently been reported that such chaperones can direct formation of the appropriate native structure of the corresponding adhesin or pilin by inserting a specific fold of the chaperone protein in place of a missing domain or helical strand of the chaperone or pilin. Thus, FimH proteins tend to have their native structure in the presence of such a chaperone but not in its absence (Choudhury et al., 1999, Science 285:1061; Sauer et al., 1999, Science 285:1058). In addition, recent publications have indicated that the required chaperone strand can be inserted into the adhesin or pilin protein, such as FimH, to provide the missing structure and produce the correct native structure.

Sokurenko et al. (1995, J. Bacteriol. 177:3680-86) had found that quantitative variations in mannose-sensitive adhesion of E. coli are due primarily to structural differences in the FimH adhesin. Further research has shown that the ability of the FimH lectins to interact with monomannosyl residues strongly correlates with their ability to mediate E. coli adhesion to uroepithelial cells so that certain phenotypic variants of type 1 fimbriae may contribute more than others to the virulence of E. coli in the urinary tract. (Sokurenko et al., 1997, J. Biol. Chem. 272:17880-6). Heretofore random point mutations in FimH genes that increase binding of the adhesin to mono-mannose residues (structures abundant in the oligosaccharide moieties of urothelial glycoproteins) had been found to confer increased virulence in the mouse urinary tract (Sokurenko et al., 1998, Proc. Natl. Acad. Sci. USA 95:8922-6).

Antibodies directed against purified whole type 1 or P pili protect against cystitis and pyelonephritis, respectively, in both murine and primate models for these diseases. See Abraham et al., 1985, Infect Immun. 48:625; Roberts et al., 1994, Proc. Natl. Acad. Sci. (USA) 91:11889; and O'Hantey et al., 1985, J. Clin. Invest. 74: 347. However, such protection is limited to either homologous E. coli strains from which the pili used as immunogens were derived, or to a small subset of serologically cross-reactive heterologous strains. Therefore, vaccines composed predominantly of the major structural proteins of pili (i.e., PapA or FimA) appear to be of limited value because antibodies developed against these highly variable proteins are specific for the strains used for immunization.

Vaccination techniques have been developed wherein the vaccine composition is delivered to the subject directly at mucosal tissues, such as gut associated lymphoid tissue, nasopharyngeal lymphoid tissue and bronchial-associated lymphoid tissue, thereby providing localized immunity. Mucosal humoral immunity has been generally thought to come from the secreted form of immunoglobulin, IgA. However, to date, there are no reports of systemic administration of a FimH vaccine composition to a primate which stimulates a humoral immune response sufficient to provide protective immunity at mucosal tissues in humans, with respect to urogenital tract infections. FimH is highly conserved not only among uropathogenic strains of E. coli, but also among a wide range of gram-negative bacteria. For example, all Enterobacteriaceae produce FimH. Thus, vaccines incorporating the FimH antigen should exhibit a broad spectrum of protection.

In addition to vaccination, inhibitory antibodies to FimE may be used in a passive immunization approach to elicit protection from infection. This type of approach has been successful used to combat respiratory syncytial virus (RSV) infection. Newborns that were given antibodies directed against RSV intravenously and intramuscularly had decreased incidence of RSV infection. This same group of investigators then examined the ability of hyperimmune serum or purified antibody to protect cotton rats and primates against RSV infection (Prince et al., 1985, Virus Res. 3:193-206; Prince et al., 1990, J. Virol. 64:3091-3092; Hemming et al, 1985, J. Infect. Dis. 152:1083-1087; Prince et al., 1983, Infect. Immun. 42:81-87; and Prince et al., 1985, J. Virol. 55:517-520). Results of these studies suggested that RSV inhibitory antibody given prophylactically inhibited respiratory tract replication of RSV in cotton rats. When given therapeutically, RSV antibody reduced pulmonary viral replication both in cotton rats and in a nonhuman primate model.

While other antigens have been utilized to produce antibodies for diagnosis and for the prophylaxis and/or treatment of bacterial urinary tract infections, there is a need for improved or more efficient vaccines and inhibitory antibodies for use in primates, and more particularly in humans. Such vaccines and inhibitory antibodies should have an improved or enhanced effect in preventing bacterial infections mediated by adhesins and pili sufficient to prevent or treat UTI in humans.

### 3. BRIEF SUMMARY OF THE INVENTION

Traditional approaches of generating antibody responses to proteins, particularly to inhibit protein function, such as binding to a binding partner, have focused on targeting antibody responses to either a conserved immunogenic linear epitope, a conformational epitope that mimics native protein structure, or a surface epitope outside of the binding site. The antibody's blocking effect results from agglutination or steric hindrance. The present invention is based, in part, on the inventors' discovery that mutant forms of the bacterial adhesin FimH, which include one or more mutations in a region of FimH critical to mannose binding, induces antibodies with a greater functional inhibitory activity (in this case binding of FimH to mannose or epithelial cells) than those antibodies induced by wild type FimH. Although not intending to be bound by any mechanism of action, the mutant FimH is predicted to adopt a more open conformation in a region critical for mannose binding such that residues that were poorly exposed in the wild type protein can be exploited as epitopes in the mutant protein. Antibodies directed to these once poorly accessible epitopes are highly inhibitory to the adhesin binding to its cellular receptor.

Accordingly, the present invention relates to methods for inducing antibodies having enhanced functional inhibitory activity, particularly enhanced ability to block binding of a protein to its binding partner, by immunization with a mutant form of the protein (i.e., having one or more amino acid modifications relative to the wild type protein or some other reference protein, which may be another mutant protein), whereby the antibodies elicited by the mutant protein have greater functional inhibitory activity than antibodies elicited by the wild-type or reference protein. In particular embodiments, the protein antigen has one or more mutations relative to the wild type or reference protein, which mutations are in regions of the protein involved in protein function (e.g., ligand or receptor binding) and which regions may be poorly exposed to solvent and/or poorly accessible for antibody production in vivo in the wild type protein. The mutations may result in exposing otherwise poorly exposed epitopes that serve as highly potent targets for functional, inhibitory antibodies. In other embodiments, the protein antigen has one or more mutations relative to the wild type protein, which mutations abolish or significantly reduce protein function (for example, but not by way of limitation, binding to a binding partner). In yet other embodiments, the protein antigen has one or more mutations relative to the wild type protein, which mutations result in a protein comprising peptides that bind more tightly to major histocompatibility complex (MHC) molecules resulting in enhanced antigen presentation.

The invention relates to production of high potency inhibitory antibodies against any protein that has a binding partner, for example, against a ligand associated with a receptor-ligand pair, particularly ligands on pathogens involved in binding to host cell receptors. Using pathogen ligands it is possible to develop vaccines that induce antibodies that inhibit binding of the pathogen to host cell receptors, thus preventing infection. Peptides and proteins that elicit antibodies with greater inhibitory activity and antibodies with greater inhibitory activity are advantageous in that they provide greater protection against infection (or whatever therapeutic or prophylactic effect is desired).

A particular embodiment of the invention provides mutant adhesin proteins and peptides that elicit antibodies that have greater activity in inhibiting binding of the adhesin protein, and/or the pathogen associated therewith, to the corresponding cellular receptor of the adhesin protein; as well as antibodies elicited by immunization with such mutant adhesin proteins and peptides. In one embodiment the adhesin molecule is PapG and the binding partner is a Galα(1-4)Gal.

In a preferred embodiment, the invention provides mutant E. coli FimH proteins and peptides that elicit antibodies that more effectively inhibit binding of FimH to mannose than antibodies elicited by wild type FimH (or even other reference mutants of FimH). In particular embodiments, the mutations involve one or more amino acid modifications (e.g., insertions, deletions and, preferably, substitutions) in the canyon region of the FimH molecule, which region is involved in mannose binding. In certain embodiments, the amino acid modifications promote a more open conformation of the FimH protein to expose regions that are poorly exposed in the wild type FimH molecule. In other embodiments, the amino acid modifications significantly reduce or abolish FimH-mannose binding. Preferably, the mutations are made in one or more of amino acid residues 1, 13, 46, 47, 48, 52, 54, 62, 67, 75, 133, 135, 137, 138, 140, 142, 154, 156, and 161 of the FimH amino acid sequence depicted in FIG. 1 and in SEQ ID NO: 4 (or the corresponding residue in a FimH variant or other adhesin molecule as determined by sequence alignment, see e.g., FIG. 3). In a preferred embodiment, the amino acid modification (preferably substitution) is at residue 54, 133, or 135 of the amino acid sequence of FimH ( FIG. 1 and SEQ ID NO: 4). In more preferred embodiments, the amino acid residue at position 54 can be substituted with asparagine or alanine; the residue at amino acid position 133 can be substituted with lysine, arginine, glutamate, or histidine; and/or the amino acid residue at position 135 can be substituted with aspartic acid. Such mutant proteins and peptides are particularly useful as vaccines for the prevention of UTI. Further, the invention encompasses molecules having two or more mutations wherein one mutation is of amino acid residue 54, 133, or 135 of the FimH amino acid sequence.

Also encompassed by the invention are vaccine compositions comprising the mutant proteins and polypeptides, and antibodies produced by immunizing with such mutant proteins and polypeptides, as well as methods of vaccination, treatment and prophylaxis using the proteins, polypeptides and antibodies of the invention.

In another embodiment, the antibodies directed against the mutant protein can be administered directly as passive immunization. The present invention is based, in part, on the development of methods for achieving or inducing a prophylactically or therapeutically effective serum titer of an antibody or fragment thereof that immunospecifically binds to a mutant antigen of a pathogen of interest in a mammal by passive immunization with such an antibody or fragment thereof. The present invention also includes the identification of antibodies with higher inhibitory activity which result in increased efficacy for prophylactic or therapeutic uses such that lower serum titers are prophylactically or therapeutically effective, thereby permitting administration of low dosages and/or less frequent administration as compared to other antibody therapeutics.

The present invention provides methods of preventing, neutralizing, treating and ameliorating one or more symptoms associated with a pathogen infection in a subject comprising administering to said subject one or more antibodies or fragments thereof which immunospecifically bind to one or more pathogen antigens and display in increased inhibitory activity. Because a lower serum titer of such antibodies or fragment thereof is therapeutically or prophylactically more effective than the effective serum titer of known antibodies, low to moderate doses of said antibodies or antibody fragments can be used to achieve a serum titer effective for the prevention, neutralization, treatment and the amelioration of symptoms associated with a pathogen infection. The use of low doses of antibodies or fragments thereof which immunospecifically bind to one or more pathogen antigens reduces the likelihood of adverse effects. Further, the increased inhibitory activity of the antibodies of the invention or fragments thereof enable less frequent administration of said antibodies or antibody fragments than previously thought to be necessary for the prevention, neutralization, treatment or the amelioration of symptoms associated with a pathogen infection.

The invention further includes co-crystals of a purified FimCH complex bound to a mannose in crystalline form. The invention encompasses the use of the three-dimensional structural representation of this co-crystal to design and/or screen mutant proteins, for example as vaccines, to produce antibodies with these mutant proteins or to design other molecules as therapeutic or prophylactic candidates for drug development. The designing or screening can be conducted using computers and computational programs or actual synthesis and in vitro and/or in vivo screening. The invention includes the use of the atomic coordinates representing the three-dimensional structure and a machine-readable medium embedded with information that corresponds to a three-dimensional structural representation of the FimCH-mannose complex.

In one aspect, the invention provides crystalline forms of polypeptides corresponding to FimCH bound to a mannose sugar. The FimCH complex of the crystalline form can be a wild type FimCH complex or a mutant FimCH complex. The mutant FimCH complex can comprise a mutant FimC or a mutant FimH or both. For example, the mutant FimCH complex can comprise a truncated mutant of FimC or a truncated mutant of FimH, or both. In certain embodiments of the invention, the mutant FimCH complex can be any mutant FimCH complex described herein. In the co-crystals, the mannose sugar can be any mannose sugar including, for example, mannopentaose, methyl-alpha-D-mannopyranoside, alpha-D-mannopyranoside, mannotriose, an oligomannoside, a dimannoside, etc.

The crystals of the invention include native crystals, in which the crystallized FimCH is substantially pure; heavy-atom derivative crystals, in which the crystallized FimCH is in association with one or more heavy-metal atoms; and co-crystals, in which the crystallized FimCH is in association with one or more compounds, including but not limited to, cofactors, ligands, substrates, substrate analogs, inhibitors, allosteric effectors, etc. to form a crystalline co-complex. Preferably, such compounds bind a catalytic or active site. The co-crystals may be native co-crystals, in which the co-complex is substantially pure, or they may be heavy-atom derivative co-crystals, in which the co-complex is in association with one or more heavy-metal atoms.

In one embodiment, wild-type FimCH alpha-D-mannopyranoside co-crystals of the invention are generally characterized by a unit cell of a=138.077+/−0.2 Å, b=138.130+/−0.2 Å, c=215.352+/−0.2 Å, α=90, β=90.005, γ=90 and are preferably of diffraction quality. In another embodiment of the invention, FimCH Q133N methyl-alpha-D-mannopyranoside co-crystals of the invention crystals of the invention are generally characterized by a unit cell of a=138.349+/−0.2 Å, b=138.334+/−0.2 Å, c=213.212+/−0.2 Å, α=90.000, β=89.983, γ=90.000 and are preferably of diffraction quality. In more preferred embodiments, the crystals of the invention are of sufficient quality to permit the determination of the three-dimensional X-ray diffraction structure of the crystalline polypeptide to high resolution, preferably to a resolution of greater than about 3 Å, typically in the range of about 1 Åto about 3 Å, about 1.5 Å to about 3 Å, or about 2 Å to about 3 Å.

The invention also provides methods of making the crystals of the invention. Generally, crystals of the invention are grown by dissolving substantially pure polypeptide in an aqueous buffer that includes a precipitant at a concentration just below that necessary to precipitate the polypeptide. Water is then removed by controlled evaporation to produce precipitating conditions, which are maintained until crystal growth ceases.

Co-crystals of the invention are prepared by soaking a native crystal prepared according to the above method in a liquor comprising the compound of the desired co-complex. Alternatively, the co-crystals may be prepared by co-crystallizing the polypeptide in the presence of the compound according to the method discussed above.

Heavy-atom derivative crystals of the invention may be prepared by soaking native crystals or co-crystals prepared according to the above method in a liquor comprising a salt of a heavy atom or an organometallic compound. Alternatively, heavy-atom derivative crystals may be prepared by crystallizing a polypeptide comprising selenomethionine and/or selenocysteine residues according to the methods described previously for preparing native crystals.

In another aspect, the invention provides machine-computer-readable media embedded with the three-dimensional structural information obtained from the crystals of the invention, or portions or substrates thereof. Such three-dimensional structural information will typically include the atomic structure coordinates of the crystallized polypeptide or co-complex, or the atomic structure coordinates of a portion thereof such as, for example, an active or binding site, but may include other structural information, such as vector representations of the atomic structures coordinates, etc. The types of machine- or computer-readable media into which the structural information is embedded typically include magnetic tape, floppy discs, hard disc storage media, optical discs, CD-ROM, electrical storage media such as RAM or ROM, and hybrids of any of these storage media. Such media further include paper on which is recorded the structural information that can be read by a scanning device and converted into a three-dimensional structure with an OCR. The machine readable media of the invention may further comprise additional information that is useful for representing the three-dimensional structure, including, but not limited to, thermal parameters, chain identifiers, and connectivity information.

The invention is illustrated by way of a working example demonstrating the crystallization and characterization of crystals, the collection of diffraction data, and the determination and analysis of the three-dimensional structure of FimCH .

The atomic structure coordinates and machine readable media of the invention have a variety of uses. For example, the coordinates are useful for solving the three-dimensional X-ray diffraction and/or solution structures of other proteins, including mutant FimCH, co-complexes comprising FimCH, and unrelated proteines, to high resolution. Structural information may also be used in a variety of molecular modeling and computer-based screening applications to, for example, intelligently design mutants of the crystallized FimCH that have altered biological activity and to computationally design and identify compounds that bind the polypeptide or a portion or fragment of the polypeptide, such as the active site.

3.1 Definitions

The term “analog” as used herein refers to a polypeptide that possesses a similar or identical function as a particular protein (e.g., a FimH polypeptide or FimCH polypeptide complex), or a fragment thereof, but does not necessarily comprise a similar or identical amino acid sequence or structure of that protein complex or a fragment thereof. A polypeptide that has a similar amino acid sequence refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of the protein or protein complex or a fragment thereof as described herein; (b) a polypeptide encoded by a nucleotide sequence that hybridizes under stringent conditions to a nucleotide sequence encoding a protein or protein complex of the invention, or fragment thereof, as described herein of at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding the protein or protein complex of the invention or a fragment thereof as described herein. A polypeptide with similar structure to a protein or protein complex of the invention or a fragment thereof as described herein refers to a polypeptide that has a similar secondary, tertiary or quaternary structure of said protein or protein complex or a fragment thereof as described herein. The structure of a polypeptide can be determined by methods known to those skilled in the art, including but not limited to, X-ray crystallography, nuclear magnetic resonance, and crystallographic electron microscopy.

The term “derivative” as used herein refers to a polypeptide that comprises an amino acid sequence of a protein (e.g., FimH) or protein complex e.g., FimCH) of the invention or a fragment thereof as described herein that has been altered by the introduction of amino acid residue substitutions, deletions or additions. The term “derivative” as used herein also refers to a protein or protein complex of the invention or a fragment thereof that has been modified, i.e., by the covalent attachment of any type of molecule to the polypeptide. For example, but not by way of limitation, a protein or protein complex or a fragment thereof may be modified, e.g., by glycosylation, acetylation, pegylation, phosphorylation, amidation, derivatization by known protecting/blocking groups, proteolytic cleavage, linkage to a cellular ligand or other protein, etc. A derivative of a protein or protein complex or a fragment thereof may be modified by chemical modifications using techniques known to those of skill in the art, including, but not limited to specific chemical cleavage, acetylation, formylation, metabolic synthesis of tunicamycin, etc. Further, a derivative of a protein or protein complex or a fragment thereof may contain one or more non-classical amino acids. A polypeptide derivative possesses a similar or identical function as a protein or protein complex or a fragment thereof described herein.

The term “fragment” as used herein refers to a peptide or polypeptide comprising an amino acid sequence of at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of a protein of the invention, such as FimH.

An “isolated” or “purified” polypeptide or polypeptide complex of the invention or fragment thereof is substantially free of cellular material or other contaminating proteins from the cell or tissue source from which the protein is derived, or substantially free of chemical precursors or other chemicals when chemically synthesized. The language “substantially free of cellular material” includes preparations of a polypeptide or polypeptide complex in which the polypeptide or polypeptide complexes separated from cellular components of the cells from which it is isolated or recombinantly produced. Thus, a polypeptide or polypeptide complex that is substantially free of cellular material includes preparations of polypeptide or polypeptide complex having less than about 30%, 20%, 10%, or 5% (by dry weight) of heterologous protein (also referred to herein as a “contaminating protein”). When the polypeptide or polypeptide complex is recombinantly produced, it is also preferably substantially free of culture medium, i.e., culture medium represents less than about 20%, 10%, or 5% of the volume of the protein preparation. When the polypeptide or polypeptide complex is produced by chemical synthesis, it is preferably substantially free of chemical precursors or other chemicals, i.e., it is separated from chemical precursors or other chemicals which are involved in the synthesis of the protein. Accordingly such preparations of the polypeptide or polypeptide complex have less than about 30%, 20%, 10%, 5% (by dry weight) of chemical precursors or compounds other than the polypeptide or polypeptide complex of interest. In a preferred embodiment, polypeptides or polypeptide complexes or fragments thereof of the invention are isolated or purified.

An “isolated” nucleic acid molecule is one which is separated from other nucleic acid molecules which are present in the natural source of the nucleic acid molecule but excludes when the nucleic acid is present as part of a cDNA library. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. “Plasmids” are designated by a lower case p preceded and/or followed by capital letters and/or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan.

The term “attachment domain” refers to the portion of a polypeptide that mediates binding between the polypeptide and a second moiety. The second moiety can comprise cell surface polypeptides and/or polysaccharides. The attachment domain for a FimH polypeptide, which is a type 1 adhesin protein produced by E. coli, is depicted in FIG. 2E.

The term “canyon region” refers to the region of the FimH polypeptide (or related adhesin) whose surface comprises residues 1, 13, 46, 47, 48, 52, 54, 133, 135, 137, 138, 140, and 142 of FimH (FIG. 2) as surface residues of the canyon structure or corresponding residues of a FimH variant or other adhesin as determined by sequence alignment and/or structural comparison.

The term “associated ligand” as used herein refers to a ligand that has an inherent function associated with the recited protein (e.g., binding, such as receptor-ligand binding) and, preferably, does not include an antigen-antibody relationship. As an example, an associated ligand to PapG is a Galα(1-4)Gal moiety. As another example, an associated ligand to FimH is a mannose moiety.

The term “periplasmic chaperone” is defined as a protein localized in the periplasm of bacteria that is capable of forming complexes with a variety of chaperone-binding proteins via recognition of a common binding epitope (or epitopes). Chaperones perform several functions. They serve as templates upon which proteins exported from the bacterial cell into the periplasm fold into their native conformations. Association of the chaperone-binding protein with the chaperone also serves to protect the binding proteins from degradation by proteases localized within the periplasm, increases their solubility in aqueous solution, and leads to their sequentially correct incorporation into an assembling pilus. Chaperone proteins are a class of proteins in gram negative bacteria that are involved in the assembly of pili by mediating such assembly, but are not incorporated into the structure. PapD is the periplasmic chaperone protein mediating the assembly of pili for P piliated bacteria and FimC is the periplasmic chaperone protein that mediates assembly of type 1 pili in bacteria.

The term “fusion protein” as used herein refers to a polypeptide that comprises an amino acid sequence of a polypeptide or fragment thereof and an amino acid sequence of a heterologous polypeptide (e.g., FimH conjugated to FimC).

The term “FimH antigen” refers to a FimH polypeptide or fragment thereof to which an antibody or antibody fragment immunospecifically binds. A FimH antigen also refers to an analog or derivative of a FimH polypeptide or fragment thereof to which an antibody or antibody fragment immunospecifically binds.

The term “FimCH complex” refers to a complex containing both a FimH and a FimC polypeptide preferably in a 1:1 ratio in the complex.

The terms “pili,” “fimbriae,” and “fibrillae” are used herein to refer to heteropolymeric protein structures located on the extracellular surface of bacteria, most commonly gram-negative bacteria. Typically these structures are anchored in the outer membrane. Throughout this specification the terms pilus, pili, fimbriae and fibrilla will be used interchangeably.

The term “substantially similar structure” as used herein refers to a mutant FimH that, although in a more open conformation, retains the general conformation of the wild type protein.

The term “antibodies or fragments that immunospecifically bind to a FimH antigen” as used herein refers to antibodies or fragments thereof that specifically bind to a FimH polypeptide or a fragment of a FimH polypeptide and do not non specifically bind to other polypeptides. Antibodies or fragments that immunospecifically bind to a FimH polypeptide or fragment thereof may have cross-reactivity with other antigens. Preferably, antibodies or fragments that immunospecifically bind to a FimH polypeptide or fragment thereof do not cross-react with other antigens. Antibodies or fragments that immunospecifically bind to a FimH polypeptide can be identified, for example, by immunoassays or other techniques known to those of skill in the art.

The term “Fab fragment” as used herein refers to a fragment of an antibody corresponding to an intact light chain associated with a V_{H}-C_{γ}1 fragment of the heavy chain. Although these fragments retain the ability to bind antigen, they are no longer bivalent and thus have lost the ability to aggregate antigen. Fab fragments may be generated by any technique known to those of skill in the art. For example, Fab fragments of the invention may be produced by proteolytic cleavage of immunoglobulin molecules using enzymes such as papain. Techniques to recombinantly produce Fab fragments can also be employed using methods known in the art such as those disclosed in PCT publication WO 92/22324; Mullinax et al., 1992, BioTechniques 12:864-869; and Sawai et al., 1995, AJRI 34:26-34; and Better et al., 1988, Science 240:1041-1043 (said references incorporated herein by reference in their entireties).

The term “functional inhibitory activity” (in some cases “inhibitory activity”) means the ability of an antibody to inhibit or reduce the binding of a protein for a binding partner. For example, the functional, inhibitory activity of an anti-FimH antibody is the ability of the antibody to inhibit or reduce the binding of FimH to a mannose moiety (e.g., mono- or tri-mannose).

The term “passive immunization” as used herein refers to the administration of immune serum or purified antibodies or fragments thereof directly to a patient. Immune serum or purified antibodies can be given prophylactically to inhibit infection or therapeutically to reduce or eliminate infection. This is distinguished from immunization of a patient with a protein to direct an in vivo immune response to produce antibodies.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleic acid positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268. modified as in Karlin and Altschul, 1993, Proc. Natl. Acad. Sci. U.S.A. 90:5873-5877. Such an algorithm is incorporated into the NBLAST and XBLAST programs of Altschul et. al., 1990, J. Mol. Biol. 215:403. BLAST nucleotide searches can be performed with the XBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score—50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (e.g., http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gasps. In calculating percent identity, typically only exact matches are counted.

The term “selenomethionine mutant” as used herein refers to a mutant which includes at least one selenomethionine (SeMet) residue, typically by substitution of a Met residue of the wild-type sequence with a SeMet residue, or by addition of one or more SeMet residues at one or both termini. Preferred SeMet mutants are those in which each Met residue is substituted with a SeMet residue.

The term “cysteine mutant” as used herein refers to a mutant in which at least one cysteine residue of the wild-type sequence is replaced with another residue, preferably with a Ser (S) residue. The term can also refer to a mutant in which a non-cysteine residue, preferably a Ser (S) residue, of the wild-type sequence is replaced with a cysteine residue.

The term “selenocysteine mutant” as used herein refers to a mutant which includes at least one selenocysteine (SeCys) residue, typically by substitution of a Cys residue of the wild-type sequence with a SeCys residue, or by addition of one or more SeCys residues at one or both termini. The term can also refer to a cysteine mutant in which at least one Cys residue is substituted with a SeCys residue. Preferred SeCys mutants are those in which each Cys residue is substituted with a SeCys residue.

The term “crystal” as used herein refers to a composition comprising a polypeptide in crystalline form. The term “crystal” includes native crystals, heavy-atom derivative crystals and co-crystals, as defined herein.

The term “native Crystal” as used herein refers to a crystal wherein the polypeptide is substantially pure. As used herein, native crystals do not include crystals of polypeptides comprising amino acids that are modified with heavy atoms, such as crystals of selenomethionine mutants, selenocysteine mutants, etc.

The term “heavy-atom derivative crystal” as used herein refers to a crystal wherein the polypeptide is in association with one or more heavy-metal atoms. As used herein, heavy-atom derivative crystals include native crystals into which a heavy metal atom is soaked, as well as crystals of selenomethionine mutants and selenocysteine mutants.

The term “co-crystal” as used herein refers to a composition comprising a co-complex, as defined above, in crystalline form. Co-crystals include native co-crystals and heavy-atom derivative co-crystals.

The term “diffraction quality crystal” as used herein refers to a crystal that is well-ordered and of a sufficient size, i.e., at least 10 μm, preferably at least 50 μm, and most preferably at least 100 μm in its smallest dimension such that it produces measurable diffraction to at least 3 Å resolution, preferably to at least 2 Å resolution and most preferably to at least 1.5 Å resolution or lower. Diffraction quality crystals include native crystals, heavy-atom derivative crystals, and co-crystals.

The term “unit cell” as used herein refers to the smallest and simplest volume element (i.e., parallelpiped-shaped block) of a crystal that is completely representative of the unit or pattern of the crystal, such that the entire crystal can be generated by translation of the unit cell. The dimensions of the unit cell are defined by six numbers: dimensions a, b and c and angles α, β and γ (Blundel et al., 1976, Protein Crystallography, Academic Press.). A crystal is an efficiently packed array of many unit cells.

The term “triclinic unit cell” as used herein refers to a unit cell in which a≠b≠c and α≠β≠γ.

The term “monoclinic unit cell” as used herein refers to a unit cell in which a≠b≠c; α=γ=90°; and β≠90°, defined to be ≧90 °.

The term “orthorhombic unit cell” as used herein refers to a unit cell in which a≠b≠c; and α=β=γ=90°.

The term “tetragonal unit cell” as used herein refers to a unit cell in which a=b≠c; and α=β=γ=90°.

The term “trigonal/rhombohedral unit cell” as used herein refers to a unit cell in which a=b=c; and α=β=γ90°.

The term “trigonal/hexagonal unit cell” as used herein refers to a unit cell in which a=b=c; α=β=90°; and γ=120°.

The term “cubic unit cell” as used herein refers to a unit cell in which a=b=c; and α=β=γ=90°.

The term “crystal lattice” as used herein refers to the array of points defined by the vertices of packed unit cells.

The term “space group” as used herein refers to the set of symmetry operations of a unit cell. In a space group designation (e.g., C2) the capital letter indicates the lattice type and the other symbols represent symmetry operations that can be carried out on the unit cell without changing its appearance.

The term “asymmetric unit” as used herein refers to the largest aggregate of molecules in the unit cell that possesses no symmetry elements that are part of the space group symmetry, but that can be juxtaposed on other identical entities by symmetry operations.

The term “crystallographically-related dimer” as used herein refers to a dimer of two molecules wherein the symmetry axes or planes that relate the two molecules comprising the dimer coincide with the symmetry axes or planes of the crystal lattice.

The term “non-crystallographically-related dimer” as used herein refers to a dimer of two molecules wherein the symmetry axes or planes that related the two molecules comprising the dimer do not coincide with the symmetry axes or planes of the crystal lattice.

The term “isomorphous replacement” as used herein refers to the method of using heavy-atom derivative crystals to obtain the phase information necessary to elucidate the three-dimensional structure of a crystallized polypeptide (Blundel et al., 1976, Protein Crystallography, Academic Press.).

The terms “multi-wavelength anomalous dispersion” or “MAD” as used herein refers to a crystallographic technique in which X-ray diffraction data are collected at several different wavelengths from a single heavy-atom derivative crystal, wherein the heavy atom has absorption edges near the energy of incoming X-ray radiation. The resonance between X-rays and electron orbitals leads to differences in X-ray scattering from absorption of the X-rays (known as anomalous scattering) and permits the locations of the heavy atoms to be identified, which in turn provides phase information for a crystal of a polypeptide. A detailed discussion of MAD analysis can be found in Hendrickson, 1985, Trans. Am. Crystallogr. Assoc., 21:11; Hendrickson et al., 1990, EMBO J. 9:1665; and Hendrickson, 1991, Science 4:91.

The terms “single wavelength anomalous dispersion” or “SAD” as used herein refers to a crystallographic technique in which X-ray diffraction data are collected at a single wavelength from a single native or heavy-atom derivative crystal, and phase information is extracted using anomalous scattering information from atoms such as sulfur or chlorine in the native crystal or from the heavy atoms in the heavy-atom derivative crystal. The wavelength of X-rays used to collect data for this phasing technique need not be close to the absorption edge of the anomalous scatterer. A detailed discussion of SAD analysis can be found in Brodersen et al., 2000, Acta Cryst., D56:431-4-1.

The terms “single isomorphous replacement with anomanous scattering” or “SIRAS” as used herein refers to a crystallographic technique that combines isomorphous replacement and anomalous scattering techniques to provide phase information for a crystal of a polypeptide. X-ray diffraction data are collected at a single wavelength, usually from a single heavy-atom derivative crystal. Phase information obtained only from the location of the heavy atoms in a single heavy-atom derivative crystal leads to an ambiguity in the phase angle, which is resolved using anomalous scattering from the heavy atoms. Phase information is therefore extracted from both the location of the heavy atoms and from anomalous scattering of the heavy atoms. A detailed discussion of SIRAS analysis can be found in North, 1965, Acta Cryst. 18:212-216; Matthews, 1966, Acta Cryst. 20:82-86.

The term “molecular replacement” as used herein refers to the method of calculating initial phases for a new crystal of a polypeptide whose structure coordinates are unknown by orienting and positioning a polypeptide whose structure coordinates are known within the unit cell of the new crystal so as to best account for the observed diffraction pattern of the new crystal. Phases are then calculated from the oriented and positioned polypeptide and combined with observed amplitudes to provide an approximate Fourier synthesis of the structure of the polypeptides comprising the new crystal. (Lattman, 1985, Methods in Enzymology 115:55-77; Rossmann, 1972, “The Molecular Replacement Method,” Int. Sci. Rev. Ser. No. 13, Gordon & Breach, New York.).

The term “having substantially the same three-dimensional structure” as used herein refers to a polypeptide that is characterized by a set of atomic structure coordinates that have a root mean square deviation (r.m.s.d.) of less than or equal to about 2 Å, or less than or equal to about 1 Å, when superimposed onto the atomic structure coordinates of Table 14 when at least about 50% to 100% of the Cα atoms of the coordinates are included in the superposition.

The term “Cα” as used herein refers to the alpha carbon of an amino acid residue.

### 4. BRIEF DESCRIPTION OF THE DRAWINGS

FIGS. 1 A-D: Wild type FimC and FimH nucleic and amino acid sequence. (A) nucleic acid sequence of FimC (SEQ ID NO: 1); (B) amino acid sequence of FimC (SEQ ID NO: 2); (C) nucleic acid sequence of FimH (SEQ ID NO: 3); (D) amino acid sequence of FimH (SEQ ID NO: 4) (from Choudhury et al. 1999, Science 285:1061 incorporated herein by reference).

FIGS. 2 A-E: Crystal structure of FimCH chaperone-adhesin complex bound to α-D-mannose. (A) Overall structure of FimCH with the two domains of the chaperone FimC (black) and the pilin domain of FimH (gray). As demonstrated previously, the receptor-binding domain of FimH is an elongated eleven-stranded β-barrel comprised of residues Phe1 to Thr158, and is connected via a flexible linker to the pilin domain of FimH. (B) The bound mannose receptor is shown at a 90° rotation of the receptor binding domain shown in (A). The mannose, the mannose-interacting residues, and the residues of the hydrophobic ridge around the pocket are shown in ball-and stick model. (C) Stereo presentation of omit electron density at 4 σ(Fₒ-F_{c}) for the α-D-mannoside bound in pocket of FimH. The interacting amino acids are shown in ball-and-stick with hydrogen bonds shown by dotted lines. (D) The receptor binding domain of FimH displaying the electrostatic potential surface, with positively and negatively charged residues shaded and hydrophobic residues labeled. (E) The tip of the FimH receptor binding domain is shown.

FIG. 3: Alignment of deduced amino acid sequences of the FimH lectin-binding domain from representative clinical isolates. The regions involved in mannose binding are shown highlighted in gray. The other positions shown were found to be heterogenous among throughout all the FimH sequences examined. The sequences that are not shown were found to be conserved also among all isolates. UTI strain J96 was used as the consensus sequence. Amino acid residues that are identical to that of J96 were indicated by “.” while the residues different from the consensus were indicated.

FIG. 4: FimH mutants were complexed with FimC, another type 1 pilus protein. Wild type FimC was found to associate with wild type FimH, the vaccine composition of wild type FimH, FimH N46A, FimH N46D, FimH Q133K, and FimH D140E equally well as assayed by ELISA using an anti-FimC antibody. closed circles=FimH N46A; open circles=FimH D140E; closed triangles=FimH Q133K; open triangles=Fim H N46D; closed squares=vaccine composition of wild type FimH; open squares=wild type FimH.

FIGS. 5 A-B: Binding of purified FimCH complexes to mono-mannose coated beads and their elution by methyl-a-D-mannopyranosides. (A) A Coomassie-stained SDS-PAGE gel shows that most FimH mutants still retained the ability to bind mono-mannose coated beads (“bound”). (A) A Coomassie-stained SDS-PAGE gel shows that bound mutant FimH proteins were eluted-off with methyl-α-D-mannopyranosides (“eluted”). (B) The ratio of bound to eluted FimH protein. Asterisk indicates no FimH was bound to the bead initially.

FIGS. 6 A-B: Binding of purified FimCH complexes to mannose as assayed by ELISA. Comparison of different mutant FimCH proteins in their ability to bind (A) mono-mannose and (B) tri-mannose. In upper panels, closed square with unbroken line=WT control, closed diamond with dotted line=N46D, closed circle=D54A, closed triangle=D54N, closed square with dashed line=S62A, opened circle=Q133K, closed upside down triangle=Q133N, closed diamond with dashed line=Q133A, half filled diamond=N135D, bottom filled square=N135A, top filled square=D140, star=D104A, and open triangle=D104E. In lower panels, closed circle and open square=WT control, open circle=I13A, closed upside down triangle=Y48A, open upside down triangle=I52A, closed square=Q133E, closed diamond=Q133H, open diamond=Q133R, filled triangle=N135D, open triangle=Y137A.

FIGS. 7 A-I: Mutant FimH expressing E. coli binding to mannose. Comparison of different mutant FimCH proteins in their ability to bind (A) monomnnose and (B) trimannose. Comparison of monomnnose and trimannose binding of PmmB66 expressing wild type FimCH with (C) untransfected PmmB66; (D) PmmB66 expressing FimCH N46A; (E) PmmB66 expressing FimCH N46D; (F) PmmB66 expressing FimCH D140E; (G) PmmB66 expressing FimCH Q133K; and (H) PmmB66 expressing FimCH S62A. (I) Mutant FimH expressing E. coli binding to control plates coated with the polyclonal anti-E. coli antibody. In panels A, B, and I, closed circle=PmmB66FimH, open circle=WT FimH, filled triangle=N46A, open triangle=N46D, closed square=D140E, open square=Q133K, diamond=S62A. In panels C-H, closed triangle=WT FimH binding to mono-mannose, open triangle=WT FimH binding to tri-mannose, closed circle=FimH binding to mono-mannose, open circle=FimH binding to tri-mannose.

FIGS. 8 A-B: Binding and invasion of 5637 cells. (A) AAEC185/pUT2002 bacteria complemented with different FimH variants did not exhibit any significant binding to 5637 cells with the exception of FimCH S62A and FimCH N46D mutants. Results were obtained from at least two different infection experiments with duplicate wells in each experiment. X-axis represents the percent cell association of total input bacteria, which includes both the surface bound and invaded bacteria. (B) Bound bacteria expressing mutant FimH proteins showed a similar degree of invasion into 5637 cells. Results shown are from one representative experiment.

FIGS. 9 A-K: Binding of type 1 piliated-bacteria to human bladder sections. AAEC185/pUT2002 bacteria complemented with (A) WT; (C) S62A; (E) N46A; (F) N46D; (H) D54A; (I) Q133A; and (J) Q133K, FimH expression and (K) vector control plasmids were used in the binding assay. Binding of (B) WT; (D) S62A; and (G) N46D can be inhibited by methyl-α-D-mannopyranosides.

FIGS. 10 A-C: Results from an ELISA of levels of anti-FimH specific IgG polyclonal antibodies in serum of vaccinated mice. Titers are shown as endpoint dilutions which are measured by an ELISA where FimH T3 (a histidine-tagged fusion protein composed of the first 165 amino acids of FimH) is the capture antigen and the detection antibody is specific to IgG. A booster immunization was given 3 weeks after the initial immunization. Doses of protein at each injection were either 4.0, 1.6, 0.64, and 0.26 μg (as indicated). Wild type FimCH was used as an immunogen for vaccination and resulting antibody titers were compared to those seen for mutant protein: (A) FimCH N46D; (B) FimCH D140E; and (C) FimCH Q133K. WT FimCH is depicted by open symbols while indicated mutant FimCH is depicted by closed symbols. square=4 ug, circle=1.6 ug, triangle=0.64 ug, diamond=0.026 ug. star=MF 59 adjuvant alone.

FIGS. 11 A-C: Hemagglutination assay inhibition by polyclonal antibodies. E. coli was preincubated with increasing dilutions of a polyclonal antibody raised against the indicated FimCH complex. The FimCH complex on the bacteria was tested for its ability to bind the mannose present on the erythrocytes in the presence of the polyclonal antibody. Decreased mean channel fluorescence in the presence of the antibody indicated that the polyclonal antibody inhibited FimCH binding in this assay. Preincubation with polyclonal antibodies raised against (A) FimCH Q133 E, FimCH Q133H, and WT FimCH and (C) FimCH N135D, FimCH Q133R, and WT FimCH inhibited bacteria binding to the erythrocytes very strongly. (B and D) Control antiserum from animals that were either not immunized or immunized with MF59 adjuvant alone showed no inhibition.

FIGS. 12 A-E: Polyclonal antibody inhibition of E. coli NU14 binding to J82 human bladder cells as measured by multiple channel fluorescence (MOF) in log2 scale. Polyclonal antibodies raised against the indicated mutant or wild type FimCH protein were preincubated with bacteria cells before addition to bladder cells for binding: (A) anti-FimCH N46D (8 week sera used after a boost at week 4); (B) anti-FimCH D140E (8 week sera used after a boost at week 4); and (C) anti-FimCH Q133K (8 week sera used after a boost at week 4). For wild type FimCH and FimCH Q133K, an additional boost at week 18 was given. Inhibitory assays were done with antisera from week 16 (darker bar) and week 20(lighterbar): (D) anti FimCH; and (E) anti-FimCH Q133K.

FIG. 13: Passive immunization with polyclonal antibodies generated with mutant FimCH protein. Mice were administered 1 mg of polyclonal antibody 4 hours prior to a large bolus challenge with E. coli Nu14. After 48 hours, mice were sacrificed to harvest the bladders. The number of CFUs were determined. A decrease in the number of CFUs indicates that the passive immunization had a protective ability.

FIG. 14: Hemagglutination assay inhibition by monoclonal antibody (MAB). E. coli was preincubated with increasing dilutions of the indicated MAB clone. The FimCH complex on the bacteria was tested for its ability to bind the mannose present on the erythrocytes in the presence of the MAB. Decreased mean channel fluorescence indicated that the MAB clone was inhibitory in this assay. Preincubation with clone 1A7 inhibited bacteria binding to the erythrocytes very strongly. Clones IC10 and 3E 1 also inhibited bacteria binding when the MABs were supplied in larger quantities. Clones 1F2, 2B2, and 1 C8 did not show an inhibitory activity.

FIG. 15: Hemagglutination assay inhibition by MAB clone 1A7. E. coli was preincubated with increasing dilutions of MAB clone 1A7. The FimCH complex on the bacteria was tested for its ability to bind the mannose present on the erythrocytes in the presence of the MAB. Decreased mean channel fluorescence indicated that the MAB clone was inhibitory in this assay. (A) Preincubation with clone 1A7 inhibited bacteria binding to the erythrocytes very strongly. (B) Controls showed that this inhibitory activity was due to preincubation with MAB clone 1A7.

FIG. 16: Tri-mannose binding inhibition by MAB. An ELISA assay was used to measure the ability of the FimCH complex on bacteria to bind tri-mannose in the presence of the MAB. A decrease in OD₄₅₀ indicated that bacteria were inhibited from binding to the tri-mannose. Both MAB clone 1A7 and 1C10 inhibited binding while MAB clone 1C8 did not closed circle=1A7, open circle=1C8, upside down triangle=1C10, triangle=anti B19 negative control.

FIG. 17: Hemagglutination assay inhibition by Fab fragments E. coli was preincubated with increasing dilutions of the indicated Fab fragment. The FimCH complex was tested for its ability to bind the mannose present on the erythrocytes in the presence of the Fab fragment. Decreased mean channel fluorescence indicates that the Fab fragment was inhibitory in this assay.

FIG. 18: Passive immunization with MABs generated with mutant FimCH protein. Mice were administered 1 mg of MAB 4 hours prior to a large bolus challenge with E. coli Nu14. After 48 hours, mice were sacrificed to harvest the bladders. The number of CFUs were determined. A decrease in the number of CFUs indicates that the passive immunization had a protective ability.

FIGS. 19 A-B: Ball-and-stick presentation of changes in the structure of the mannose binding pocket between (A) wild type FimCH and (B) Q133N FimCH. Hydrogen bonds are shown as dotted lines and aromatic contacts are shown as dashed lines. Water molecules are labeled as W1 and/or W2.

### 5. DETAILED DESCRIPTION OF THE INVENTION

The present invention is based, in part, on the inventors' discovery that certain mutant forms of the bacterial adhesin FimH, which have one or more mutations in a canyon region of FimH critical to mannose binding, induced antibodies with a greater functional inhibitory activity (in this case inhibiting binding of FimH to mannose or epithelial cells) than those antibodies induced by wild type FimH. Although not intending to be bound by any mechanism of action, the mutant FimH is predicted to adopt a more open conformation in a region critical for mannose binding such that residues that were poorly exposed in the wild type protein can be exploited as epitopes in the mutant protein. Antibodies directed to these once inaccessible epitopes are highly inhibitory to the adhesin.

Accordingly, the present invention relates to methods for inducing antibodies having enhanced functional inhibitory activity, particularly enhanced ability to block binding of a protein to its binding partner, by immunization with a mutant form of the protein (i.e., having one or more amino acid modifications relative to the wild type protein or some other related reference protein, which may be another mutant protein), whereby the antibodies elicited by the mutant protein have greater functional inhibitory activity than antibodies elicited by the wild-type or reference protein. In particular embodiments, the protein antigen has one or more mutations relative to the wild type or reference protein, which mutations are in regions of the protein involved in protein function (e.g., ligand or receptor binding) and which regions may be poorly exposed to solvent and/or poorly accessible for antibody production in vivo in the wild type protein. The mutations may result in exposing otherwise buried epitopes that serve as highly potent targets for functional, inhibitory antibodies. In other embodiments, the protein antigen has one or more mutations relative to the wild type protein, which mutations abolish or significantly reduce protein function (for example, but not by way of limitation, binding to a binding partner). In yet other embodiments, the protein antigen has one or more mutations relative to the wild type protein or reference protein, which mutations result in a protein comprising peptides that bird more tightly to MHC molecules resulting in enhanced antigen presentation.

The invention relates to production of high potency inhibitory antibodies against any protein that has a binding partner, for example, against a ligand associated with a receptor-ligand pair, particularly ligands on pathogens involved in binding to host cell receptors. Using pathogen ligands is it possible to develop vaccines that induce antibodies that inhibit binding of the pathogen to host cell receptors, thus preventing infection. Additionally, the antibodies directed against the pathogen protein can be administered directly as passive immunization. Peptides and proteins that elicit antibodies with greater inhibitory activity and antibodies with greater inhibitory activity are advantageous in that they provide greater protection against infection (or whatever therapeutic or prophylactic effect is desired).

Each of the above-described peptides and proteins can be designed or generated using information from the complex of FimCH-mannose in crystalline form, such information includes but is not limited to the three-dimensional structure. Thereafter, antibodies to the novel mutant peptides or proteins can be generated.

5.1 Mutant Proteins as Antigens for High Potency Inhibitory Antibodies

The present invention relates to methods for inducing antibodies having enhanced functional inhibitory activity, particularly enhanced ability to block binding of a protein to its binding partner, by immunization with a mutant form of the protein (i. e., having one or more amino acid modifications relative to the wild type protein or some other related reference protein, which may be another mutant protein), whereby the antibodies elicited by the mutant protein have greater functional inhibitory activity than antibodies elicited by the wild-type or reference protein.

In particular embodiments, the protein antigen has one or more mutations relative to the wild type or reference protein, which mutations are in regions of the protein involved in protein function (e.g., ligand or receptor binding) and which regions are poorly exposed to solvent and/or poorly accessible for antibody production in vivo in the wild type protein. The mutations may result in exposing otherwise poorly exposed epitopes that serve as highly potent targets for functional, inhibitory antibodies. Such residues can be identified by any means known in the art, preferably, by computer modeling, to identify residues critical for a particular protein conformation, which residues, when modified (preferably, substituted with another amino acid residue), result in a more open protein conformation. In preferred embodiments, the more open protein conformation exposes or one or more regions of the protein that are poorly exposed in the wild type or reference protein, more preferably, these one or more regions are involved (in some aspects, critical for) protein binding to a binding pair. Preferably, the amino acid residue that is substituted differs in hydrophobicity, polarity, size, or charge from the amino acid present at that position in the wild type or reference protein. Additionally, libraries of random mutants can be generated at one or more residues identified by modeling or other methods to be critical for protein conformation, particularly in regions important in protein binding to a binding partner (e.g., ligand binding to an associated receptor), and/or the mutation of which is predicted to expose otherwise poorly exposed regions, preferably those involved in protein binding. Such libraries of randomly mutated proteins can be screened using methods well known in the art for mutant proteins that elicit antibodies that have higher functional inhibitory activity than the antibodies elicited by a wild type or reference protein.

In other embodiments, the protein antigen has one or more mutations (i.e., amino acid modifications) relative to the wild type protein, which mutations abolish or significantly reduce protein function (for example, but not by way of limitation, binding to a binding partner). The residues to be mutated can be identified by any method known in the art for identifying residues critical for ligand binding, for example, but not by way of limitation, protein modeling and mutational analysis. Preferably, the amino acid residue that is substituted differs in hydrophobicity, polarity, size, or charge from the amino acid present at that position in the wild type or reference protein. Additionally, libraries of random mutants can be generated at one or more residues identified by modeling or other methods to be critical for ligand binding. Such libraries of randomly mutated protein can be screened for mutant proteins that have reduced or no binding activity and/or the ability to elicit antibodies that have higher functional inhibitory activity than the antibodies elicited by a wild type or reference protein.

In yet other embodiments, the protein antigen has one or more mutations relative to the wild type protein, which mutations result in a protein comprising peptides that bind more tightly to MHC molecules resulting in enhanced antigen presentation.

The mutant proteins of the invention may have any number of mutations relative to the corresponding wild type protein or reference protein as long as they elicit antibodies that have greater functional inhibitory activity than antibodies elicited by the wild type or reference protein. In certain embodiments, the protein contains 1, 2, 3, 4, 5, 6, 7, 8, 9, 10, 15, 20 or more than 25 mutations. In certain embodiments, the protein also contains mutations relative to the wild type or reference protein that do not affect (or even decrease) the ability of the protein to elicit antibodies with a greater functional inhibitory activity than those elicited by the wild type or reference protein, as long as the mutant protein is able to elicit such high potency inhibitory antibodies. The invention also includes fragments of the mutant proteins that elicit antibodies with greater inhibitory activity than the wild type or reference protein and/or than the corresponding fragment of the wild type or reference protein.

The invention relates to producing mutants of any protein that is a member of a binding pair, including proteins that bind non-protein molecules, such as carbohydrates including lectins, lipids, steroids, non-peptide hormones, or other small molecules. In particular, such proteins are members of a ligand-receptor pair. Either the ligand or the receptor may be the antigen that is mutated. Such mutated ligand or receptor can then be used to raise antibodies with enhanced activity to block ligand-receptor binding. In a preferred embodiment, the binding pair is not an antigen-antibody binding pair.

In preferred embodiments, the invention relates to methods for inducing antibodies having enhanced functional inhibitory activity, particularly enhanced ability to block binding of a pathogenic protein to its host cell receptor, by immunization with a mutant form of the pathogenic protein (i.e., having one or more amino acid modifications relative to the wild type or reference protein), whereby the antibodies elicited by the mutant pathogenic protein have greater functional inhibitory activity than antibodies elicited by the wild-type protein. In particular embodiments, the pathogenic protein antigen has one or more mutations relative to the wild type or reference pathogenic proteins which mutations result in exposing regions of the protein which are poorly exposed to solvent and/or not accessible for antibody production in vivo in the wild type protein. By way of example but not limitation, the mutations may result in exposing otherwise poorly exposed epitopes that serve as highly potent targets for antibodies that inhibit binding of pathogenic proteins to host cell receptors.

A particular embodiment of the invention provides methods for inducing antibodies having enhanced ability to block binding of a parasitic ligand to its host cell receptor, by immunization with a mutant form of the parasitic ligand (i.e., having one or more amino acid modifications relative to the wild type or reference ligand), whereby the antibodies elicited by the mutant ligand have greater functional inhibitory activity than antibodies elicited by the wild-type or reference ligand. In particular embodiments, the parasitic ligand has one or more mutations relative to the wild type or reference parasitic ligand, which mutations result in exposing regions which are poorly exposed to solvent and/or poorly accessible for antibody production in vivo in the wild type ligand.

Highly preferred embodiments of the invention provide methods for inducing antibodies having enhanced ability to block binding of a microbial adhesin protein to its host cell receptor, by immunization with a mutant form of the adhesin protein, which mutants induce of antibodies with greater inhibitory activity than antibodies elicited by the wild-type adhesin protein. In particular embodiments, the adhesin protein has one or more mutations relative to the wild type or a reference adhesin, which mutations result in exposing regions of the protein which are poorly exposed in the wild type protein. In other embodiments, the mutations significantly reduce or abolish binding of the adhesin to its host cell surface receptor.

Accordingly, the present invention also relates to antibodies that target protein binding interactions including but not limited to examples such as antibodies that target αVβ3 integrin, FimH, FimCH, and RSV. Embodiments provide antibodies that immunospecifically bind a member of a binding pair. The binding pair can be any two molecules that specifically interact with each other. In specific embodiments, the one member of the binding pair is an antigen of an infectious disease agent (i.e., a molecule on the surface of an infectious disease agent) or a cellular receptor for an infectious disease agent. Such antigens of infectious disease agents include FimH of E. coli, and antigens of HSV-2, gonococcus, Treponema pallidum, Chlamydia trachomatis or human papillomavirus The first member of the binding pair can also be a cancer antigen (i.e., a molecule expressed on the surface of a cancer cell). Such cancer antigens include human milk fat globule antigen (HMFG), an epitope of polymorphic epithelial mucin antigen (PEM), or a human colon carcinoma-associated protein antigen.

The invention further provides methods of treatment or prevention using the antibodies of the invention as discussed herein. For example, peptides to elicit antibodies or antibodies directed to an infectious agent or a cellular receptor for an infectious disease agent or a cancer antigen can be used in the treatment or prevention of an infectious disease or a cancer associated with the expression of the particular antigen of the infectious disease agent or the cellular receptor for the infectious disease agent.

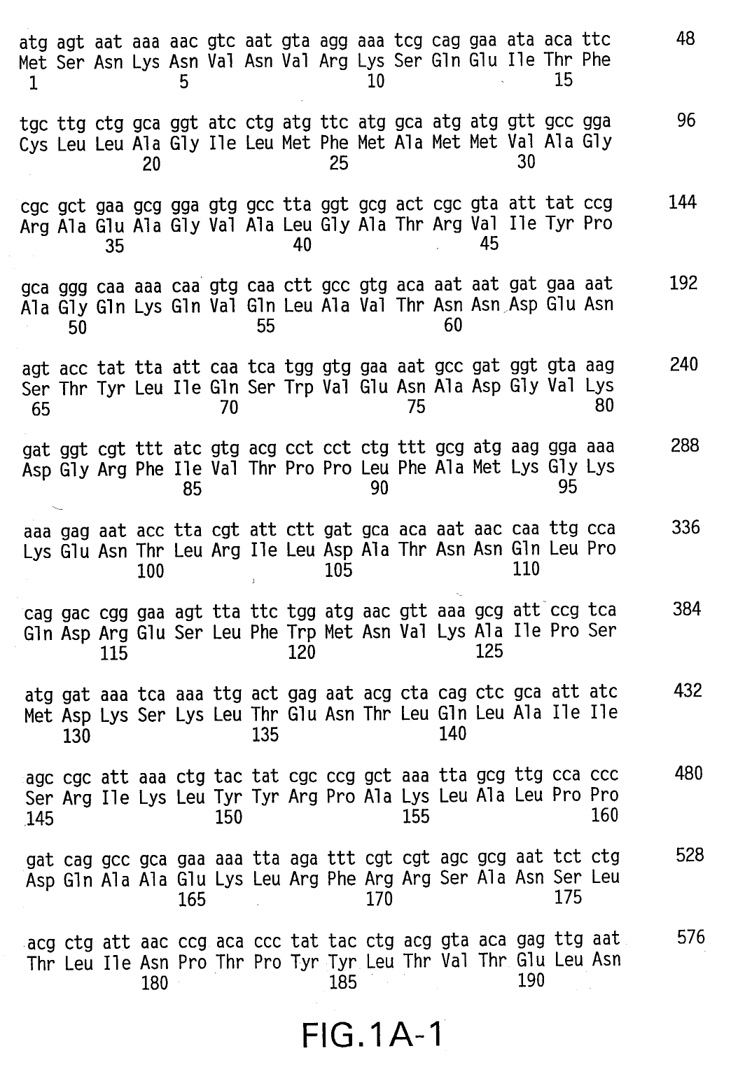

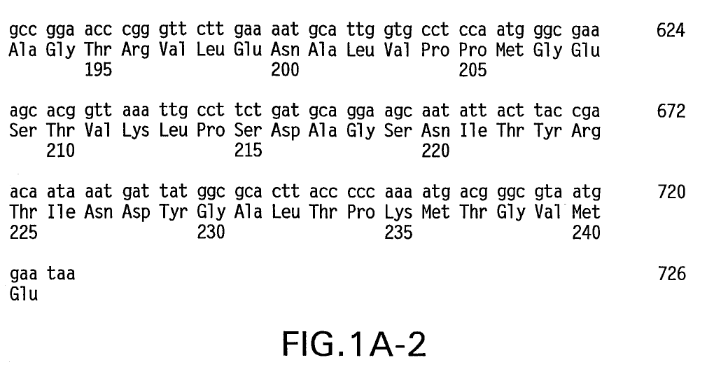

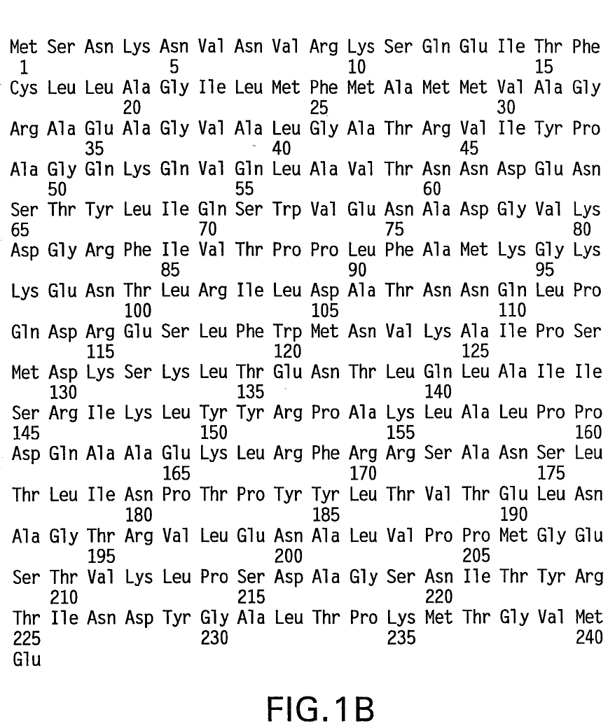

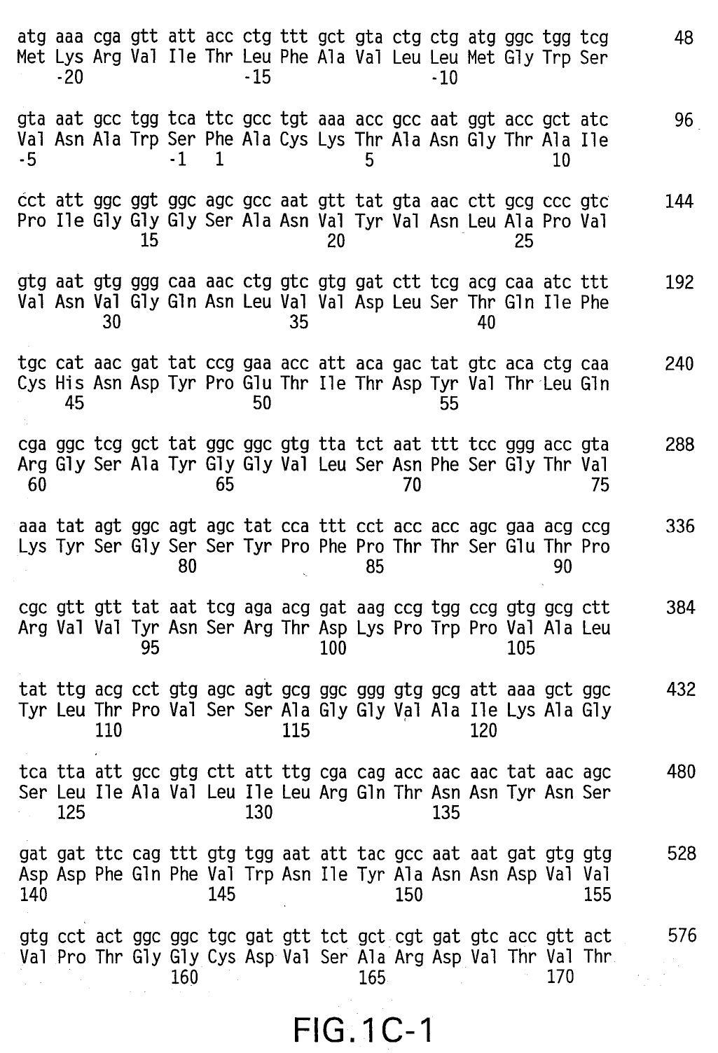

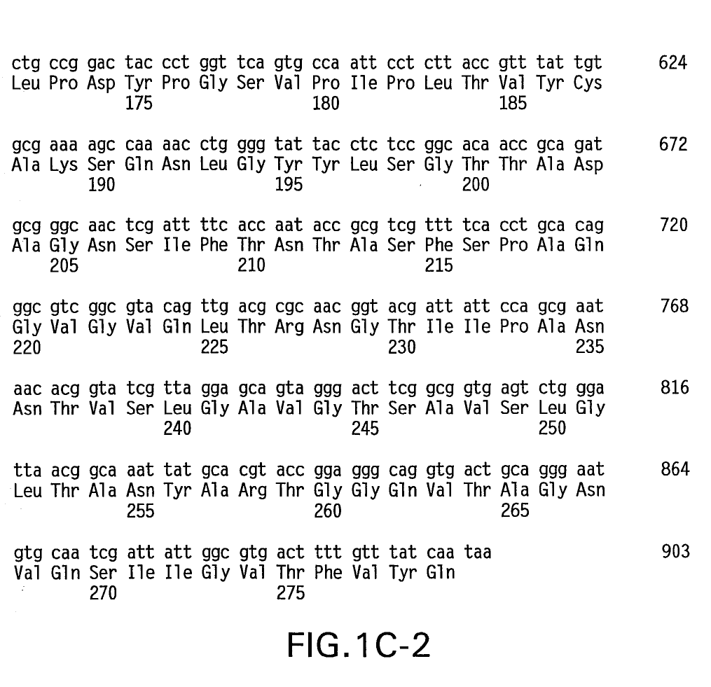

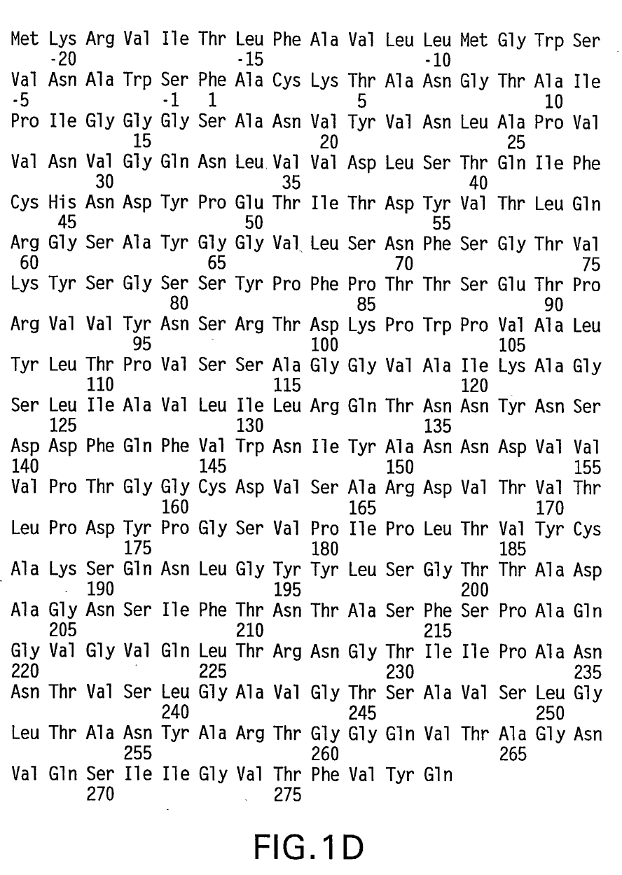

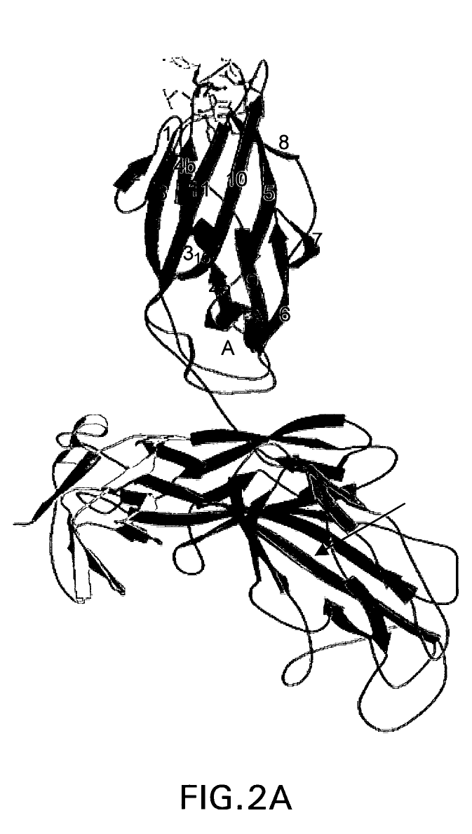

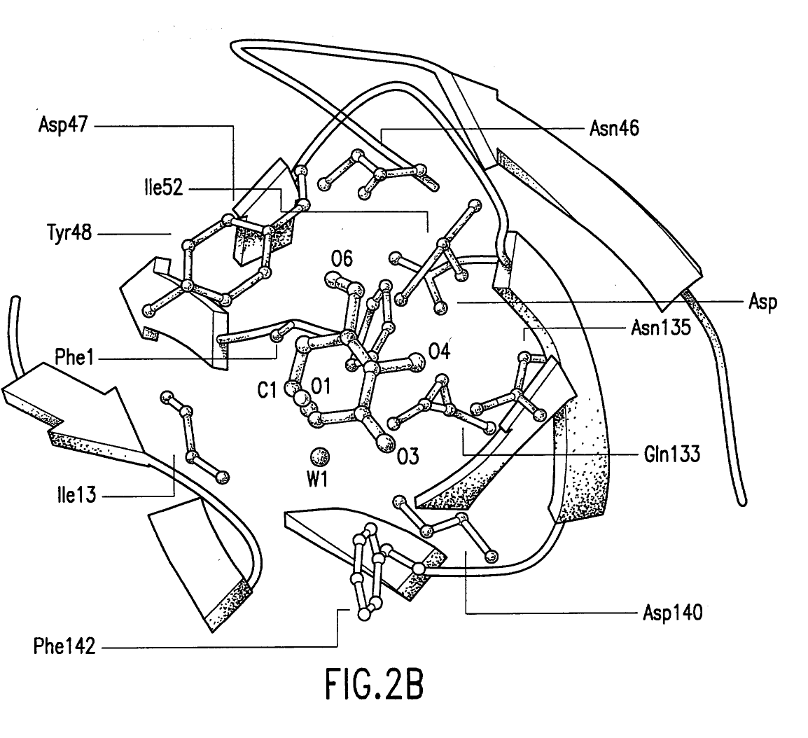

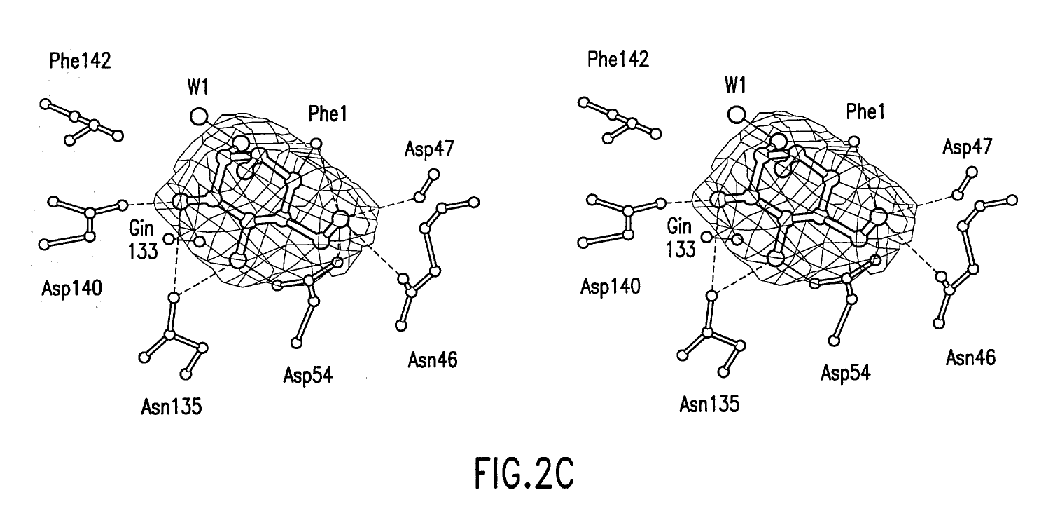

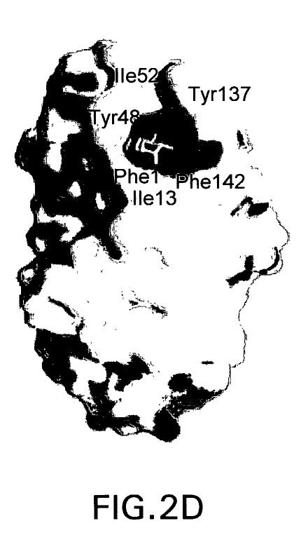

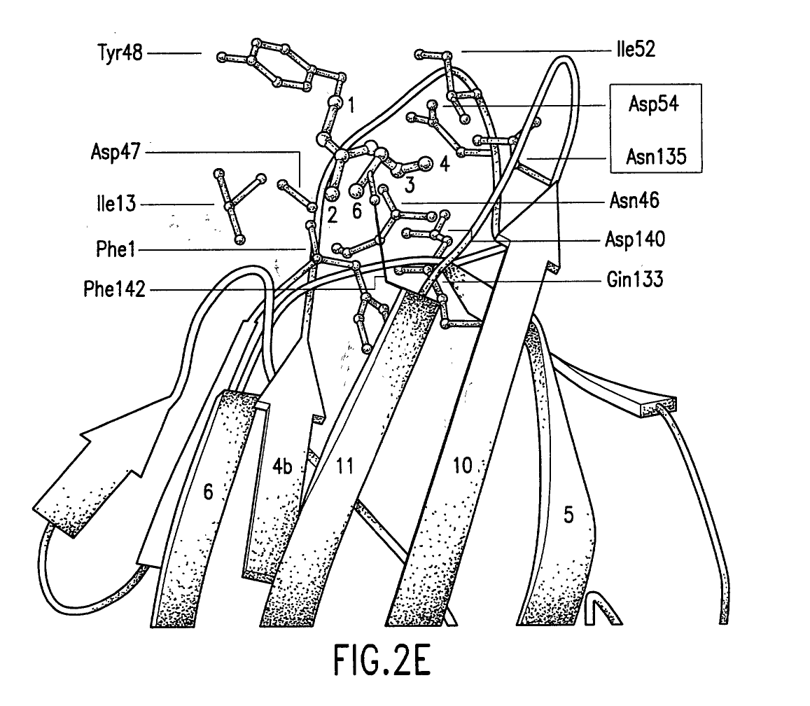

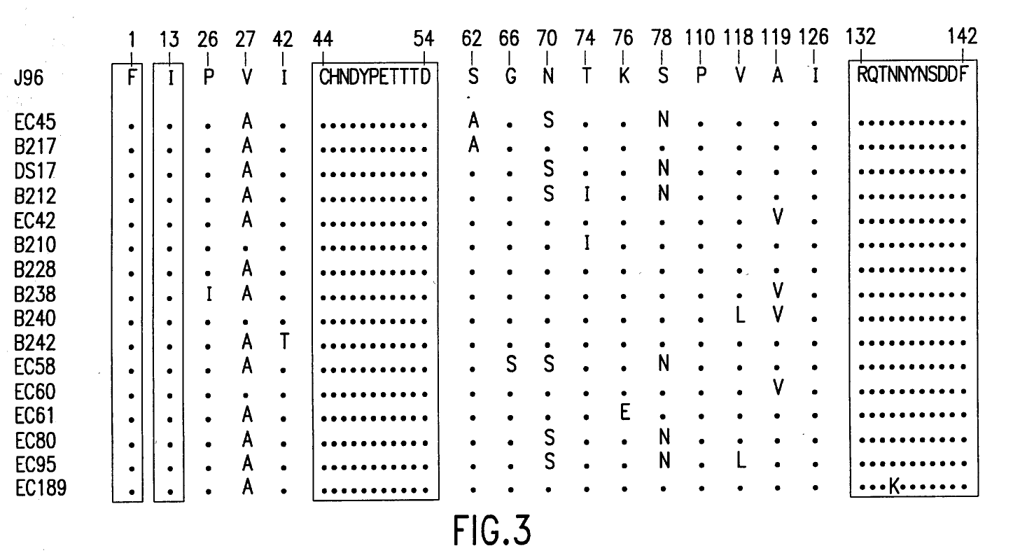

In a preferred embodiment of the invention, antibodies to mutant adhesin proteins are generated to inhibit binding of adhesins to cellular receptors. In particular, FimH proteins are responsible for the adhesin binding of type 1 pili to bladder epithelial cells. Accordingly, the invention provides mutant forms of FimH (relative to the FimH amino acid sequence of FIG. 1 (SEQ ID NO: 3) or corresponding FimH variant of FIG. 3) or other bacterial adhesin (e.g., PapG) that elicit antibodies that have greater inhibitory activity (that prevents binding of the bacteria or the isolated adhesin to the cellular receptor (mannose moieties in the case of FimH) or host cell (bladder epithelial cells in the case of FimH) than antibodies elicited by wild type or a reference FimH or other bacterial adhesin. Without being limited by theory, the invention provides mutant forms of FimH in which the canyon region of FimH, which is involved in mannose binding, adopts a more open conformation, exposing regions that are poorly exposed in wild type FimH. FimH residues involved in maintaining the canyon structure and/or that, when mutated, would result in exposing poorly exposed regions in the wild type FimH may be identified by any method known in the art. For example, such residues may be identified by protein modeling. The crystal structure for the FimCH complex is depicted in Choudhury et al 1999, Science 285:1061-1066, which is hereby incorporated by reference in its entirety. More importantly, the crystal structure of the mannose binding pocket of FimH has been determined by co-crystallizing a highly purified FimCH chaperone-adhesin complex together with D-mannose (see FIG. 2).

In other embodiments, mutant FimH proteins, or other bacterial adhesins, are provided where one or more amino acid modifications are introduced into the FimH protein that significantly reduce or abolish binding of FimH to mannose or the other bacterial adhesin to its cell surface receptor. In either embodiment, the residues to be modified may be identified through protein modeling and/or analysis of site specific on naturally occurring or any other mutants to identify residues that, when mutated, alter protein structure or binding of the protein to its cellular receptor. In certain embodiments, libraries of mutant adhesins having random mutations at one or more residues are screened for mutant adhesins in which poorly exposed mutant regions are exposed, mutant adhesins that lack or have significantly reduced binding to the cellular receptor, and/or mutant adhesins that can elicit antibodies that have greater functional inhibitory activity than antibodies elicited by the wild type or reference adhesin.

In preferred embodiments, the mutant protein of the invention is a mutant FimH protein having one or more amino acid modifications (preferably substitutions) at one or more of residues 1, 2, 3, 4, 10, 11, 12, 13, 14, 15, 16, 42, 43, 44, 45, 46, 47, 48, 49, 50, 51, 52, 53, 54, 55, 56, 57, 58, 59, 77, 78, 89, 90, 91, 92, 93, 94, 95, 96, 97, 98, 99, 100, 101, 102, 103, 104, 105, 130, 131, 132, 133,134, 135, 136, 137, 138, 139, 140, 141, 142, 143, 144, 145 or 146 of the FimH amino acid sequence in FIG. 1 (SEQ ID NO: 3) (the residue numbers discussed herein all refer to the residues as numbered on the FimH sequence of FIG. 1, unless specifically noted and intend to include corresponding residues in a variant of FimH, as determined by sequence alignment with the amino acid sequence in FIG. 1). In a more preferred embodiment, the amino acid modifications (preferably substitutions) are made at one or more of residues 1, 2, 13, 44, 45, 46, 47, 48, 49, 50, 51, 52, 53, 54, 55, 56, 57, 77, 78, 101, 131, 132, 133, 134, 135, 136, 137, 138, 139, 140, 141, 142, 143 or 144 of the amino acid sequence of FimH in FIG. 1 (SEQ ID NO: 3). In yet another embodiment, the amino acid modifications (preferably substitutions) are made at one or more of residues 1, 45, 46, 47, 52, 53, 54, 55, 56, 93, 94, 95, 133, 134 or 135 of the amino acid sequence of FimH ( FIG. 1). In another embodiment, the amino acid modifications (preferably substitutions) are made at one or more of residues 1, 3, 44, 54, 133, 135, 140, 142 and 144 of the amino acid sequence of FimH ( FIG. 1). In a preferred embodiment, the amino acid modification (preferably substitution) is at residue 54, 133, or 135 of the amino acid sequence of FimH ( FIG. 1), more preferably where the residue at position 54, 133, or 135 is substituted with a charged residue (in other embodiments substituted with an amino acid having greater steric effects than the wild type residue). In more preferred embodiments, the amino acid residue at position 54 can be substituted with asparagine or alanine; the residue at amino acid position 133 can be substituted with lysine, arginine, glutamate, or histidine; and/or the amino acid residue at position 135 can be substituted with aspartic acid. In other embodiments, the FimH amino acid modifications are in canyon region of FimH, preferably where the canyon region has a surface of residues 1, 13, 46, 47, 48, 52, 54, 133, 135, 137, 138, 140and 142.

In one embodiment, the site of one or more of the amino acid modifications occurs at a residue that interacts with mannose e.g., as determined by molecular modeling using the crystal structure provided in FIG. 2, or the crystal structure in Choudhury et al. 1999, (Science 285:1061-1066, incorporated by reference herein in its entirety) or both. Further, the mutations can similarly be made by modeling based upon related crystal structures such as that disclosed herein as FIG. 2 and in U.S. application Ser. No. 09/637,216 filed Aug. 11, 2000, entitled “Anti-Bacterial Compounds Directed vs Pilus Biogenesis, Adhesion and Activity; Co-crystals of Pilus Subunits and Methods of Use” by Hultgren et al., which is herein incorporated by reference.

For example, the modification is made at one or more residues 1, 46, 47, 54, 133, 135, 140, and 142 of FimH (SEQ ID NO: 3), which interact with mannose as shown in Table 1.

TABLE 1
FimH Amino Acid Residues Which Interact with Mannose

| residue position | amino acid residue |
|---|---|
| 1 | phenylalanine (F) |
| 46 | asparagine (N) |
| 47 | aspartic acid (D) |
| 54 | aspartic acid (D) |
| 133 | glutamine (Q) |
| 135 | asparagine (N) |
| 140 | aspartic acid (D) |
| 142 | phenylalanine (F) |

In another embodiment, the site of one or more of the amino acid modifications occurs within the hydrophobic ring surrounding the mannose-binding pocket of FimH. For example, residues 13, 48, 52, and 142 of FimH (SEQ ID NO: 3), as shown in Table 2.

TABLE 2
FimH Amino Acid Residues of the Hydrophobic Ring

| residue position | amino acid residue |
|---|---|
| 13 | isoleucine (I) |
| 48 | tyrosine (Y) |
| 52 | isoleucine (I) |
| 142 | phenylalanine (F) |

In one embodiment, the site of one or more of the amino acid modifications occurs within about 15 angstroms from the a carbon residue 54 of FimH, e.g., as determined by molecular modeling using the crystal structure provided in FIG. 2 and in Choudhury et al. 1999, (Science 285:1061-1066, incorporated by reference herein in its entirety). For example, the modification is made at one or more residues 1, 2, 3, 4, 10, 11, 12, 13, 14, 15, 16, 42, 43, 44, 45, 46, 47, 48, 49, 50, 51, 52, 53, 54, 55, 56, 57, 58, 59, 77, 78, 89, 90, 91, 92, 93, 94, 95, 96, 97, 98, 99, 100, 101, 102, 103, 104, 105, 130, 131, 132, 133, 134, 135, 136, 137, 138, 139, 140, 141, 142, 143, 144, 145 and 146 of FimH (SEQ ID NO: 3). (see Table 3)

TABLE 3
Residues 15 angstroms from the α carbon of residue 54 in FimH

| residue position | wild type amino acid |
|---|---|
| 1 | phenylalanine (F) |
| 2 | alanine (A) |
| 3 | cysteine (C) |
| 4 | lysine (K) |
| 10 | alanine (A) |
| 11 | isoleucine (I) |
| 12 | proline (P) |
| 13 | isoleucine (I) |
| 14 | glycine (G) |
| 15 | glycine (G) |
| 16 | glycine (G) |
| 42 | isoleucine (I) |
| 43 | phenylalanine (F) |
| 44 | cysteine (C) |
| 45 | histidine (H) |
| 46 | asparagine (N) |
| 47 | aspartic acid (D) |
| 48 | tyrosine (Y) |
| 49 | proline (P) |
| 50 | glutamic acid (E) |
| 51 | asparagine (N) |
| 52 | isoleucine (I) |
| 53 | threonine (T) |
| 54 | aspartic acid (D) |
| 55 | tyrosine (Y) |
| 56 | valine (V) |
| 57 | threonine (T) |
| 58 | leucine (L) |
| 59 | glutamine (Q) |
| 78 | serine (S) |
| 89 | glutamic acid (E) |
| 90 | threonine (T) |
| 91 | proline (P) |
| 92 | arginine (R) |
| 93 | valine (V) |
| 94 | valine (V) |
| 95 | tyrosine (Y) |
| 96 | asparagine (N) |
| 97 | serine (S) |
| 98 | arginine (R) |
| 99 | threonine (T) |
| 101 | lysine (K) |
| 102 | proline (P) |
| 103 | tryptophan (W) |
| 104 | proline (P) |
| 105 | valine (V) |
| 130 | isoleucine (I) |
| 131 | leucine (L) |
| 132 | arginine (R) |
| 133 | glutamine (Q) |
| 134 | threonine (T) |
| 135 | asparagine (N) |
| 136 | asparagine (N) |
| 137 | tyrosine (Y) |
| 138 | asparagine (N) |
| 139 | serine (S) |
| 140 | aspartic acid (D) |
| 141 | aspartic acid (D) |
| 142 | phenylalanine (F) |
| 143 | glutamine (Q) |
| 144 | phenylalanine (F) |
| 145 | valine (V) |
| 146 | tryptophan (W) |

In another embodiment, the site of one or more of the amino acid modifications occurs within about 10 angstroms from the a carbon residue 54 of FimH. For example, residues 1, 2, 13, 44, 45, 46, 47, 48, 49, 50, 51, 52, 53, 54, 55, 56, 57, 77, 78, 101, 131, 132, 133, 134, 135, 136, 137, 138, 139, 140, 141, 142, 143 and 144 of FimH (SEQ ID NO: 3) (see Table 4)

TABLE 4
Residues 10 angstroms from the α carbon of residue 54 in FimH

| residue position | wild type amino acid |
|---|---|
| 1 | phenylalanine (F) |
| 2 | alanine (A) |
| 13 | isoleucine (I) |
| 44 | cysteine (C) |
| 45 | histidine (H) |
| 46 | asparagine (N) |
| 47 | aspartic acid (D) |
| 48 | tyrosine (Y) |
| 49 | proline (P) |
| 50 | glutamic acid (E) |
| 51 | asparagine (N) |
| 52 | isoleucine (I) |
| 53 | threonine (T) |
| 54 | aspartic acid (D) |
| 55 | tyrosine (Y) |
| 56 | valine (V) |
| 57 | threonine (T) |
| 91 | proline (P) |
| 92 | arginine (R) |
| 93 | valine (V) |
| 94 | valine (V) |
| 95 | tyrosine (Y) |
| 96 | asparagine (N) |
| 97 | serine (S) |
| 98 | arginine (R) |
| 99 | threonine (T) |
| 101 | lysine (K) |
| 131 | leucine (L) |
| 132 | arginine (R) |
| 133 | glutamine (Q) |
| 134 | threonine (T) |
| 135 | asparagine (N) |
| 136 | asparagine (N) |
| 137 | tyrosine (Y) |
| 138 | asparagine (N) |
| 139 | serine (S) |
| 140 | aspartic acid (D) |
| 141 | aspartic acid (D) |
| 142 | phenylalanine (F) |
| 143 | glutamine (Q) |
| 144 | phenylalanine (F) |

In another embodiment, the site of one or more of the amino acid modifications occurs within about 5 angstroms from the a carbon of residue 54 of FimH. For example, the modification is at one or more of residues 1, 45, 46, 47, 52, 53, 54, 55, 56, 93, 94, 95, 133, 134 and 135 of FimH (SEQ ID NO: 3). (see Table 5)

TABLE 5
Residues 5 angstroms from the α carbon of residue 54 in FimH

| residue position | wild type amino acid |
|---|---|
| 1 | phenylalanine (F) |
| 45 | histidine (H) |
| 46 | asparagine (N) |
| 47 | aspartic acid (D) |
| 52 | isoleucine (I) |
| 53 | threonine (T) |
| 54 | aspartic acid (D) |
| 55 | tyrosine (Y) |
| 56 | valine (V) |
| 93 | valine (V) |
| 94 | valine (V) |
| 95 | tyrosine (Y) |
| 133 | glutamine (Q) |
| 134 | threonine (T) |
| 135 | asparagine (N) |

In another embodiment, the amino acid modifications are made within 15, 10 and 5 angstroms of the α-carbon of residues 1, 13, 46, 47 48, 54, 133, 135, 140 or 142 of the FimH binding domain.

5.2 Prophylactic and Therapeutic Uses

The present invention encompasses methods of treatment and prophylaxis and therapies which involve administering mutant proteins or polypeptides to an animal, preferably a mammal, and most preferably a human, for preventing, treating, or ameliorating symptoms associated with a disease, disorder, or infection. Prophylactic and therapeutic compounds of the invention include, but are not limited to, mutant proteins, polypeptides, antibodies elicited by the mutant proteins and polypeptides and nucleic acids encoding the proteins and antibodies. Proteins and antibodies may be provided in pharmaceutically acceptable compositions as known in the art or as described herein.

Methods of the invention include methods of treatment and prophylaxis involving administration of a mutant polypeptide or protein of the invention that elicits high potency inhibitory antibodies that inhibit or reduce protein binding, particularly where the protein binding is relevant to some disease or disorder. For example, peptides which elicit antibodies and the resulting antibodies which disrupt or prevent the interaction between an antigen and its binding partner may be administered to an animal, preferably a mammal and most preferably a human, to treat, prevent or ameliorate one or more symptoms associated with infection.

In a specific embodiment, the methods of the invention produce antibodies that prevent a viral or bacterial antigen from binding to its binding partner (e.g., host cell receptor) by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to antigen binding to its host cell receptor in the absence of said antibodies.

Peptides and proteins that elicit antibodies which do not prevent a viral or bacterial antigen from binding its host cell receptor but inhibit or downregulate viral or bacterial replication can also be administered to an animal to treat, prevent or ameliorate one or more symptoms associated with a viral or bacterial infection. The ability of an antibody to inhibit or downregulate viral or bacterial replication may be determined by techniques described herein or otherwise known in the art. For example, the inhibition or downregulation of viral replication can be determined by detecting the viral titer in the animal.

Examples of pathogen host cell receptor interactions that may be disrupted in methods of the invention include, but are not limited to, those in Table 6.

TABLE 6

| Pathogen | Cellular Receptor |
|---|---|
| B-lymphotropic papovavirus (LAV) | LAV receptor on B-cells |
| Bordetella pertussis | Adenylate cyclase |
| Borna Disease virus (BDV) | BDV surface glycoproteins |
| Bovine coronavirus | N-acetyl-9-O-acetylneuraminic acid receptor |
| Choriomeningitis virus | CD4+ |
| Dengue virus | Highly sulphated type Heparin sulphate |
| | p65 |
| E. coli | Galα(1-4)Gal-containing receptors |
| | mannose-containing receptors |
| Ebola | CD16b |
| Echovirus 1 | Integrin VLA-2 receptor |
| Echovirus-11 (EV) | EV receptor |
| Endotoxin (LPS) | CD14 |
| Enteric bacteria | Glycoconjugate receptors |
| Enteric Orphan virus | alpha/beta T-cell receptor |
| Enteroviruses | Decay-accelerating factor receptor |
| Feline leukemia virus | Extracellular envelope glycoprotein (Env-SU) receptor |
| Foot and mouth disease virus | Immunoglobulin Fc receptorPoxvirusM-T7 |
| Gibbon ape leukemia virus | GALV receptor |
| (GALV) | |
| Gram-negative bacteria | CD14 receptor |
| Heliobacter pylori | Lewis(b) blood group antigen receptor |
| Hepatitis B virus (HBV) | T-cell receptor |
| Herpes Simplex Virus | Heparin sulphate glycoaminoglycan receptor |
| | Fibroblast growth factor receptor |
| HIV-1 | CC-Chemokine receptor CCR5 |
| | CD11a |
| | CD2 |
| | G-protein coupled receptor |
| | CD4 |
| Human cytomegalovirus | Heparin sulphate proteoglycan |
| | Annexin II |
| | CD13 (aminopeptidase N) |
| Human coronovirus | Human aminopeptidase N receptor |
| Influenza A, B & C | Hemagglutinin receptor |
| Legionella | CR3 receptor |
| | Protein kinase receptor |
| | Galactose N-acetylgalactosamine (Gal/GalNAc)- |
| | inhibitable lectin receptor |
| | Chemokine receptor |
| Leishmania mexicana | Annexin I |
| Listeria monocytogenes | ActA protein |
| Measles virus | CD46 receptor |
| Meningococcus | Meningococcal virulence associated Opa receptors |
| Morbilliviruses | CD46 receptor |
| Mouse hepatitis virus | Carcinoembryonic antigen family receptors |
| | Carcinoembryonic antigen family Bgla receptor |
| Murine leukemia virus | Envelope glycoproteins |
| Murine gamma herpes virus | gamma interferon receptor |
| Murine retrovirus | Glycoprotein gp70 |
| | Rmc-1 receptor |
| Murine coronavirus mouse | Carcinoembryonic antigen family receptors |
| hepatitis virus | |
| Mycobacterium avium-M | Human Integrin receptor alpha v beta 3 |
| Neisseria gonorrhoeae | Heparin sulphate proteoglycan receptor |
| | CD66 receptor |
| | Integrin receptor |
| | Membrane cofactor protein |
| | CD46 |
| | GM1 |
| | GM2 |
| | GM3 |
| | CD3 |
| | Ceramide |
| Newcastle disease virus | Hemagglutinin-neuraminidase protein |
| | Fusion protein |
| Parvovirus B19 | Erythrocyte P antigen receptor |
| Plasmodium falciparum | CD36 receptor |
| | Glycophorin A receptor |
| Pox Virus | Interferon gamma receptor |
| Pseudomonas | KDEL receptor |
| Rotavirus | Mucosal homing alpha4beta7 receptor |
| Samonella typhiurium | Epidermal growth factor receptor |
| Shigella | α5β1 integrin protein |
| Streptococci | Nonglycosylated J774 receptor |
| T-helper cells type 1 | Chemokine receptors including: |
| | CXCR1-4 |
| | CCR1-5 |
| | CXCR3 |
| | CCR5 |
| T-cell lymphotropic virus 1 | gp46 surface glycoprotein |
| Vaccinia virus | TNFRp55 receptor |
| | TNFRp75 receptor |
| | Soluble Interleukin-1 β receptor |

In a specific embodiment, an antibody inhibits or downregulates viral or bacterial replication by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to viral or bacterial replication in absence of said antibody.

Proteins and peptides that elicit antibodies and the resulting antibodies can also be used to prevent, inhibit or reduce the growth or metastasis of cancerous cells. In a specific embodiment, an antibody inhibits or reduces the growth or metastasis of cancerous cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the growth or metastasis in absence of said antibody. Examples of cancers include, but are not limited to, leukemia (e.g., acute leukemia such as acute lymphocytic leukemia and acute myclocytic leukemia), neoplasms, tumors (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumors, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, eminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma), heavy chain disease, metastases, or any disease or disorder characterized by uncontrolled cell growth.

Proteins and peptides that elicit antibodies and antibodies can also be used to reduce the inflammation experienced by animals, particularly mammals, with inflammatory disorders. In a specific embodiment, an antibody reduces the inflammation in an animal by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the inflammation in an animal in the not administered said protein, peptide or antibody. Examples of inflammatory disorders include, but are not limited to, rheumatoid arthritis and asthma, Peptides, proteins and antibodies of the invention can also be used to prevent the rejection of transplants. Antibodies can also be used to prevent clot formation. Further, peptides and proteins that elicit antibodies and antibodies that function as agonists of the immune response can also be administered to an animal, preferably a mammal, and most preferably a human, to treat, prevent or ameliorate one or more symptoms associated with the disease, disorder, or infection.

The compositions of this invention may also be advantageously utilized in combination with other monoclonal or chimeric antibodies, or with lymphokines or hematopoietic growth factors (such as, e.g., IL-2, IL-3, IL-7, and IL-9, which, for example, serve to increase the number or activity of effector cells which interact with the antibodies. The antibodies of this invention may also be advantageously utilized in combination with other monoclonal or chimeric antibodies, or with lymphokines or hematopoietic growth factors (such as, e.g., IL-2, IL-3, IL-7, and IL-9), which, for example, serve to increase the immune response. The compositions of this invention may also be advantageously utilized in combination with one or more drugs used to treat a disease, disorder, or infection such as, for example anti-cancer agents, anti-inflammatory agents anti-viral agents, or antibiotics. Examples of anti-cancer agents include, but are not limited to, isplatin, ifosfamide, paclitaxel, taxanes, topoisomerase I inhibitors (e.g., CPT-11, topotecan, 9-AC, and GG-2 11), gemcitabine, cisplatin, doxinedria, vinorelbine, oxaliplatin, 5-fluorouracil (5-FU), leucovorin, vinorelbine, temodal, and taxol. Examples of anti-viral agents include, but are not limited to, cytokines (e.g., IFN-α, IFN-β, IFN-γ), inhibitors of reverse transcriptase (e.g., AZT, 3TC, D4T, ddC, ddI, d4T, 3TC, adefovir, efavirenz, delavirdine, revirapine, abacavir, and other dideoxynucleosides or dideoxyfluoronucleosides), inhibitors of viral mRNA capping, such as ribavirin, inhibitors of proteases such HIV protease inhibitors (e.g., amprenavir, indinavir, nelfinavir, ritonavir, and saquinavir,), amphoteripin, in B, castanospermine as an inhibitor of glycoprotein processing, inhibitors of neuraminidase such as influenza virus neuraminidase inhibitors (e.g., zanamivir and oseltamivir), topoisomerase I inhibitors (e.g., camptothecins and analogs thereof), amantadine, and ramantadine. Examples of anti-inflammatory agents include, but are not limited to, nonsteroidal anti-inflammatory drugs such as COX-2 inhibitors (e.g., meloxicam, celecoxib, rofecoxib, flosulide, and SC-58635, and MK-966), ibuprofen and indomethacin, and steroids (e.g., deflazacort, dexamethasone and methylprednisolone).

In a specific embodiment, antibodies administered to animal are of a species origin or species reactivity that is the same species as that of the animal. Thus, in a preferred embodiment, human or humanized antibodies, or nucleic acids encoding human or human, are administered to a human patient for therapy or prophylaxis.

In a preferred embodiment, the present invention encompasses the administration of a mutant bacterial adhesin protein or fragment thereof preferably associated with a pathogenic bacteria. The mutant bacterial adhesin protein is preferably a type 1 pilus polypeptide. Fragments of the bacterial adhesin protein containing, for example, all or an immunogenic portion of the mutant attachment domain (preferably, a portion that binds cell surface residues and/or mannose) of the protein may also be administered. Such bacterial adhesin proteins also include analogs, homologs and variants thereof, preferably that retain decrease binding activity. In other embodiments, the mutant bacterial adhesin proteins are provided as part of a complex, for example, with a bacterial chaperone protein, as detailed below.

In preferred embodiments, the methods of the invention encompass administration of a mutant FimH protein, including variants, derivatives analogs and fragments thereof, preferably variants, derivatives, analogs and fragments that have decreased mannose binding activity and, preferably, are immunogenic. In one embodiment of the present invention, recombinantly produced mutant FimH proteins (as well as functional analogs) from bacteria that produce type 1 pili are contemplated.

In additional preferred embodiments, the methods of the invention encompass administration of an antibody or antigen binding fragment thereof directed to the mutant proteins that have inhibitory functions with respect to the infective properties of the pathogen (e.g., prevent binding of the pathogen to its cellular receptor). In one embodiment of the present invention, recombinantly produced antibodies are contemplated.

In preferred embodiments, the invention provides methods of treating or preventing a bacterial infection, particularly a urogenital tract infection, more particularly a UTI, caused by a gram negative bacterium of the family Enterobacteriaceae, especially E. coli. In other embodiments, the infection is caused by Staphylococcus saprophyticus or Staphylococcus aureus, Klebsiella spp, Proteus spp, Serratia spp, or Pseudomonas spp. In an alternative embodiment, the infection is caused by infection with unusual organisms such as parasites, e.g., Echinococcus, Schistosoma haematobium or mansoni, protozoa, e.g., Trichomonas, yeast such as Candida spp, Blastomyces spp, or Coccidio des immitis, or acid-fast organisms such as Mycobacterium tuberculosis. In preferred embodiments, the infection to be treated or prevented using the methods of the invention is a UTI, a bladder infection, a kidney infection, pyelonephritis, cystitis, and asymptomatic bacteriuria.

In one embodiment, the primate is a human. In another embodiment, the human subject is susceptible to a recurrence of UTI due to having had a prior UTI, particularly having had two, three or even more UTIs in one year, or has a familial susceptibility, e.g., genetic predisposition. In other embodiments, the human subject is pregnant and/or hospitalized, or is immuno-compromised due, for example, to a secondary disease, such as HIV or cancer, or having undergone therapies therefor, has an HIV infection or has a cancer, or is in remission therefrom. In a specific embodiment, the human subject has asymptomatic bactourea and, in particular embodiments, also is diabetic and/or is a pregnant woman. Reduced levels of IL-6 and/or IL-8 as compared to the normal levels of IL-6 and IL-8 in pregnant women have been correlated with difficulty in clearing urinary tract infections. Thus, the invention further includes treatment of pregnant women with reduced levels of IL-6 and/or IL-8. In another specific embodiment, the subject is at risk of developing end stage renal disease; accordingly, the invention further provides a method for preventing progression to end stage renal disease.

In a preferred embodiment, the compositions of the invention are administered parenterally, preferably via intramuscular, intravenous or subcutaneous injection; orally; transdermally; nasally; muscosally, including vaginaly, rectally, buccally, preferably the mucosal delivery is via a vaginal suppository; and finally via pulmonary delivery. Preferably, the compositions are not injected intraperitoneally The polypeptides and antibodies of the present invention may also be present in the form of a composition. Such compositions, where used for pharmaceutical purposes, will commonly have the polypeptide of the present invention suspended in a pharmacologically acceptable diluent or excipient, or they may be in lyophilized form. The methods of the invention encompass administering an effective amount of composition to elicit sufficient levels of antibodies, particularly IgGs, in serum and, preferably, in mucosal secretions, such as urine and/or genital secretions, to prevent bacterial infection, e.g., to reduce the incidence of such bacterial infections, or to treat or ameliorate the symptoms of bacterial infection.

5.3 Pharmaceutical Formulations and Administration of Mutant Proteins

The mutant polypeptides and fragments thereof described in herein are useful immunogens for preparing pharmaceutical compositions that stimulate the production of antibodies that inhibit the interaction of binding partners. This antibody inhibition is greater than that of antibodies raised against the corresponding non-mutant polypeptides.

The antibodies of the invention can be directed to any protein that has a binding partner. In preferred embodiments, the antibodies have enhanced functional inhibitory activity to block binding of a pathogenic protein to its host cell receptor. A particular embodiment of the invention provides antibodies having enhanced ability to block binding of a parasitic ligand to its host cell receptor. Highly preferred embodiments of the invention provide antibodies having enhanced ability to block binding of a microbial adhesin protein to its host cell receptor. In the most preferred embodiment, the microbial adhesion protein is FimH.

The pharmaceutical compositions useful herein also contain a pharmaceutically acceptable carrier, including any suitable diluent or excipient, which includes any pharmaceutical agent that does not itself induce the production of antibodies harmful to the primate receiving the composition, and which may be administered without undue toxicity.

In preferred embodiments, the pharmaceutical formulations of the invention comprise a FimH polypeptide (preferably, mutant FimH polypeptide of the invention), FimCH polypeptide complex (preferably where the FimH component is a mutant FimH of the invention) or fragments or variants thereof, and a pharmaceutically acceptable carrier or excipient. Pharmaceutically acceptable carriers include but are not limited to saline, buffered saline, dextrose, water, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co., N.J. current edition). The formulation should suit the mode of administration . In a preferred embodiment, the formulation is suitable for administration to humans, preferably is sterile, non-particulate and/or non-pyrogenic. In a preferred embodiment the pharmaceutical composition contains a citrate buffer, preferably, about 20 mM sodium citrate and 0.2 M NaCl, more preferably with a pH of 6.0, and an adjuvant, such as MF59C.1 (Chiron, Emeryville, Calif.).

The composition, if desired, can also contain minor amounts of wetting or emulsifying agents, or pH buffering agents. The composition can be a solid form, such as a lyophilized powder suitable for reconstitution, a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile diluent can be provided so that the ingredients may be mixed prior to administration.

The invention provides in one embodiment a thermally stable and/or chemically stable pharmaceutical composition that is suitable for reconstitution into an injectable sterile and particulate-free solution.

The invention also provides a pharmaceutical pack or kit comprising one or more containers filled with one or more of the ingredients of the vaccine formulations of the invention. In a preferred embodiment, the kit comprises two containers one containing the adhesin protein or protein complex and the other containing an adjuvant. Associated with such container(s) can be a notice in the form prescribed by a governmental agency regulating the manufacture, use or sale of pharmaceuticals or biological products, which notice reflects approval by the agency of manufacture, use or sale for human administration.

The invention also provides that mutant polypeptide, or polypeptide complex or fragments thereof are packaged in a hermetically sealed container such as an ampoule or sachette indicating the quantity of composition. In one embodiment, the composition is supplied as a liquid, in another embodiment, as a dry sterilized lyophilized powder or water free concentrate in a hermetically sealed container and can be reconstituted, e.g., with water or saline to the appropriate concentration for administration to a subject. Preferably, the composition is supplied as a dry sterile lyophilized powder in a hermetically sealed container at a unit dosage of preferably, 1 μg, 5 μg, 10 μg, 20 μg, 25 μg, 30 μg, 50 μg, 75 μg, 100 μg, 123 μg, 150 μg, or 200 μg. Alternatively, the unit dosage of the composition is less than 1 μg, (for example 0.5 μg or less, 0.25 μg or less, or 0.1 μg or less), or more than 123 μg, (for example 150 μg or more, 250 μg or more, or 500 μg or more).

The composition should be administered within 12 hours preferably within 6 hours, within 5 hours, within 3 hours, or within 1 hour after being reconstituted from the lyophylized powder.

In an alternative embodiment, a mutant polypeptide or fragment thereof is supplied in liquid form in a hermetically sealed container indicating the quantity and concentration of the polypeptide composition. Preferably, the liquid form of the mutant polypeptide or fragment thereof is supplied in a hermetically sealed container at least 50 μg/ml, more preferably at least 100 μg/ml, at least 200 μg/ml, at least 500 μg/ml, at least 1 mg/ml, and most preferably 490 μg/ml.

In a preferred embodiment, mutant polypeptide is stored in a 3 ml sterile vial containing 1.0 ml of vaccine formulated in 500 μg/ml of mutant polypeptide in 20 mM sodium citrate, 0.2 M NaCl at a pH of 6.0. In this formulation, the vial should contain a clear colorless liquid. The adjuvant is stored in a separate 3 ml vial containing 0.7 ml of adjuvant (MF59C.1; 39 mg/ml squalene, 4.7 mg/ml each Tween 80 and Span 85, 10 mM citrate in sterile water for injection at pH 6.5) and is typically a cloudy, white, turbid liquid. The diluent is supplied in another separate 3 ml vial containing 2.0 ml of 20 mM sodium citrate, 0.2 M NaCl at a pH of 6.0. The diluent is a clear, colorless liquid. Each of these vials should be stored in a refrigerator (2° C. to 8° C./36° F. to 46° C.).

In a preferred embodiment, the mutant polypeptide is prepared for injection into a subject immediately prior to the injection, i.e., mixed with diluent and adjuvant.

Doses of 1 μg, 5 μg, 25 μg and 123 μg of mutant polypeptide are preferably prepared for administration as follows:

For a 1 μg dose, gently invert several times one mutant polypeptide vaccine vial, three diluent vials and one adjuvant vial and let stand at room temperature for twenty minutes. Withdraw 0.5 ml from the vaccine vial into a 1.0 ml syringe and inject into a diluent vial. Immediately mix by gently swirling. Withdraw 0.5 ml using a new needle and inject into a second diluent vial. Immediately mix by gently swirling. Withdraw 0.5 ml using a new needle and inject into the third diluent vial. Immediately mix by gently swirling. Withdraw 0.7 ml using a new needle and inject into the adjuvant vial. Immediately mix by gently inverting the vial 5-10 times. Withdraw 0.7 ml into a new 1.0 ml syringe using a new needle. Disconnect the needle used to draw up the drug, attach a sterile 23 gauge, one inch needle for administration to the subject, and adjust the final volume in the syringe to 0.5 ml (eject any extra through the needle), label syringe and place in the labeled zip-lock bag. This 0.5 ml dose will contain approximately 1 μg of mutant polypeptide and MF59C.1 (approximately 10 mg squalene) in 15 mM sodium citrate and 0.1 M NaCl.

For a 5 μg dose, gently invert several times one vaccine vial, three diluent vials and one adjuvant vial and let stand at room temperature for twenty minutes. Withdraw 0.5 ml using a new needle and inject into a second diluent vial. Immediately mix by gently swirling. Withdraw 0.5 ml using a new needle and inject into the third diluent vial. Immediately mix by gently swirling. Withdraw 0.7 ml using a new needle and inject into the adjuvant vial. Immediately mix by gently inverting the vial 5-10 times. Withdraw 0.7 ml into a new 1.0 ml syringe using a new needle. Disconnect the needle used to draw up the drug, attach a sterile 23 gauge, one inch needle for administration to the subject, and adjust the final volume in the syringe to 0.5 ml (eject any extra through the needle), label syringe and place in the labeled zip-lock bag. This 0.5 ml dose will contain approximately 5 μg of the mutant polypeptide and MF59C.1 (approximately 10 mg squalene) in 15 mM sodium citrate and 0.1 M NaCl.

For a 25 μg dose, gently invert several times one vaccine vial, three diluent vials and one adjuvant vial and let stand at room temperature for twenty minutes. Withdraw 0.5 ml using a new needle and inject into the third diluent vial. Immediately mix by gently swirling. Withdraw 0.7 ml using a new needle and inject into the adjuvant vial. Immediately mix by gently inverting the vial 5-10 times. Withdraw 0.7 ml into a new 1.0 ml syringe using a new needle. Disconnect the needle used to draw up the drug, attach a sterile 23 gauge, one inch needle for administration to the subject, and adjust the final volume in the syringe to 0.5 ml (eject any extra through the needle), label syringe and place in the labeled zip-lock bag. This 0.5 ml dose will contain approximately 25 μg of the mutant polypeptide and MF59C.1 (approximately 10 mg squalene) in 15 mM sodium citrate and 0.1 M NaCl.

For a 123 μg dose, gently invert several times one vaccine vial, three diluent vials and one adjuvant vial and let stand at room temperature for twenty minutes. Withdraw 0.7 ml using a new needle and inject into the adjuvant vial. Immediatey mix by gently inverting the vial 5-10 times. Withdraw 0.7 ml into a new 1.0 ml syringe using a new needle. Disconnect the needle used to draw up the drug, attach a sterile 23 gauge, one inch needle for administration to the subject, and adjust the final volume in the syringe to 0.5 ml (eject any extra through the needle), label syringe and place in the labeled zip-lock bag. This 0.5 ml dose will contain approximately 123 μg of the mutant polypeptide , and MF59C.1 (approximately 10 mg squalene) in 15 mM sodium citrate and 0.1 M NaCl.

In another specific embodiment, 1, 5, 25 or 123 μg of the mutant polypeptide in 0.5 ml of MF59C.1, as prepared above, is injected slowly, i.e., 20 to 30 seconds, into the deltoid muscle of the upper arm of the subject at day 0, followed by a booster dose approximately one month, and a second booster, if necessary approximately 4-6 months, after the initial administration. The necessity of booster shots can be determined by measuring serum, urine or mucosal secretions for immunoglobulins specific to the polypeptide injected.

5.3.1 Adjuvants

The invention encompasses mutant proteins e.g., FimH compositions, for use in vaccines administered in conjunction with adjuvants, wherein the adjuvants can be mixed (before or simultaneously upon injection) with the mutant polypeptide composition or alternatively the adjuvant is not mixed with the mutant polypeptide composition but is separately co-administered with the mutant polypeptide composition.

Mutant polypeptide compositions are administered with one or more adjuvants. In one embodiment, the mutant polypeptide composition is administered together with a mineral salt adjuvants or mineral salt gel adjuvant. Such mineral salt and mineral salt gel adjuvants include, but are not limited to, aluminum hydroxide (ALHYDROGEL, REHYDRAGEL), aluminum phosphate gel, aluminum hydroxyphosphate (ADJU-PHOS), and calcium phosphate.

In another embodiment, the mutant polypeptide composition is administered with an immunostimulatory adjuvant. Such class of adjuvants, include, but are not limited to, cytokines (e.g., interleukin-2, interleukin-7, interleukin-12, granulocyte-macrophage colony stimulating factor (GM-CSF), interferon-γ, interleukin-1β (IL-1β), and IL-1β peptide or Sclavo Peptide), cytokine-containing liposomes, triterpenoid glycosides or saponins (e.g., QuilA and QS-21, also sold under the trademark STIMULON, ISCOPREP), Muramyl Dipeptide (MDP) derivatives, such as N-acetyl-muramyl-L-threonyl-D-isoglutamine (Threonyl-MDP, sold under the trademark TERMURTIDE), GMDP, N-acetyl-nor-muramyl-L-alanyl-D-isoglutamine, N-acetylmuramyl-L-alanyl-D-isoglutaminyl-I-alanine-2-(1′-2′-dipalmitoyl-sn-glycero-3-hydroxyphosphoryloxy)-ethylamine, muramyl tripeptide phosphatidylethanolamine (MTP-PE), unmethylated CpG dinucleotides and oligonucleotides, such as bacterial DNA and fragments thereof, LPS, monophosphoryl Lipid A (3D-MLA sold under the trademark MPL), and polyphosphazenes.

In another embodiment, the adjuvant used is a CpG adjuvant. Oligo-deoxynucleotides (ODN) containing unmethylated CpG dinucleotides within specific sequence contexts (CpG motifs) are detected, like bacterial or viral DNA, as a danger signal by the vertebrate immune system. CpG ODN synthesized with a nuclease-resistant phosphorothioate backbone have been shown to be a potent Th1-directed adjuvant in mice. In addition, an ODN with a TpC dinucleotide at the 5′ end followed by three 6 mer CpG motifs (5′-GTCGTT-3′) separated by TpT dinucleotides has shown high immunostimulatory activity for human, chimpanzee, and rhesus monkey leukocytes (Hartmann et al., 2000, J. Immun, 164:1617-1624).

In another embodiment, suitable adjuvants include, but are not limited to: aluminim hydroxide, N-acetyl-muramyl-L-threonyl-D-isoglutamine (thr MDP), -acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine.

In another embodiment, the adjuvant used is a particulate adjuvant, including, but not limited to, emulsions, e.g., squalene or squaline oil-in-water adjuvant formulations, such as SAF and MF59, e.g., prepared with block-copolymers, such as L-121 (polyoxypropylene/polyoxyethylene) sold under the trademark PLURONIC L-121, Liposomes, Virosomes, cochleates, and immune stimulating complex, which is sold under the trademark ISCOM. In a preferred embodiment, the adjuvant is MF59, MF59C or most preferably MF59C.1 (Chiron, Emeryville, Calif.) or a derivative thereof. Freund's Complete Adjuvant and Freund's Incomplete Adjuvant are also commonly used adjuvants in test animals, however these adjuvants are less preferred in primates, in particular for use in humans.

In another embodiment, a microparticulate adjuvant is used. Microparticulate adjuvants include, but are not limited to biodegradable and biocompatible polyesters, homo-and copolymers of lactic acid (PLA) and glycolic acid (PGA), poly(lactide-co-glycolides) (PLGA) microparticles, polymers that self-associate into particulates (poloxamer particles), soluble polymers (polyphosphazenes), and virus-like particles (VLPs) such as recombinant protein particulates, e.g., hepatitis B surface antigen (HbsAg).

Yet another class of adjuvants that may be used include mucosal adjuvants, including but not limited to heat-labile enterotoxin from Escherichia coli (LT), cholera holotoxin (CT) and cholera Toxin B Subunit (CTB) from Vibrio cholerae, mutant toxins (e.g. LTK63 and LTR72), microparticles, and polymerized liposomes. Additional examples of mucous targeting adjuvants are E. coli mutant heat-labile toxin LT's with reduced toxicity, live attenuated organisms that bind M cells of the gastrointestinal tract, such as V. cholera and Salmonella typhi, Mycobacterium bovis (BCG), in addition to mucosal targeted particulate carriers such as phospholipid artificial membrane vesicles, copolymer microspheres, lipophilic immune-stimulating complexes and bacterial outer membrane protein preparations (proteosomes).

In other embodiments, any of the above classes of adjuvants may be used in combination with each other or with other adjuvants. For example, non limiting examples of combination adjuvant preparations that can be used to administer the FimH compositions of the invention include liposomes containing immunostimulatory protein, cytokines, or T-cell and/or B-cell peptides, or microbes with or without entrapped IL-2 or microparticles containing enterotoxin. Other adjuvants known in the art are also included within the scope of the invention (Vaccine Design: The Subunit and Adjuvant Approach. Chap. 7, Michael F. Powell and Mark J. Newman (eds.), Plenum Press, New York, 1995, which is incorporated herein in its entirety).

The effectiveness of an adjuvant may be determined by measuring the induction of specific antibodies directed against the FimH composition formulated with the particular adjuvant. In a preferred embodiment, the adjuvant MF59C.1 is mixed with the vaccine composition, and MF59C.1 is at a dose of approximately 10 mg squalene, in 15 mM sodium citrate and 0.1 M NaCl.

5.3.2 Vaccine Administration

The invention provides methods of treatment, prophylaxes, and amelioration of one or more symptoms associated with pathogen infection by administering to a subject of an effective amount of a vaccine preparation comprising a protein of the invention or fragment thereof. The subject is preferably a mammal such as non-primate (e.g., cows, pigs, horses, cats, dogs, rats etc.) and a primate (e.g., monkey such as a cynomolgous monkey and a human). In a preferred embodiment, the subject is a human. In specific embodiments, the subject is a woman. The antibodies are particularly useful in women priviously infected with UTI, pregnant women, and sexually active women. Finally, women previously infected with sexually transmitted diseases or otherwise at risk of UTI are recipients of the antibodies of the invention. In another embodiment, the subject is a diabetic, preferably a diabetic woman. In another embodiment, diabetic subjects can be vacciniated with WT FimCH.

Vaccines are generally administered parenterally using methods known in the art, however, many methods of administration may be used including but not limited to oral, intradermal, intramuscular, intravenous, subcutaneous, transdermal, intranasal routes, via pulmonary delivery, via suppository ( e.g., vaginal suppository), via scarification (scratching through the top layers of skin, e.g., using a bifurcated needle). In a preferred embodiment, the vaccine is administered intramuscularly. In yet another embodiment administration is not intraperitoneal due to the substantial risks of first pass hepatic removal of the polypeptides and also because of risk of infection and adhesions.

Various delivery vehicles are known and can be used to administer the mutant polypeptide compositions of the invention or fragments thereof, e.g., encapsulation in liposomes, microparticles, microcapsules, recombinant cells capable of expressing the mutant polypeptide compositions, receptor-mediated endocytosis (see, e.g., Wu and Wu, 1987, J. Biol. Chem. 262:4429-4432), construction of a nucleic acid as part of a retroviral or other vector, for example, the pCGA139-1-1 vector as described herein which can be administered as a DNA vaccine or alternatively, the nucleic acid vector can be introduced into a host cell such that the host cell expresses and secretes the vaccine composition, e.g., the mutant polypeptide complex, and the host cell is subsequently implanted into the subject contained within a membrane suitable for human implantation.

Methods of administering a polypeptide or fragment thereof, or pharmaceutical composition include, but are not limited to, parenteral administration (e.g., intradermal, intramuscular, intravenous and subcutaneous), epidural, mucosal (e.g., intranasal and oral or pulmonary routes or by vaginal suppositories), and topically. In a specific embodiment, compositions of the present invention or fragments thereof are administered intramuscularly, intravenously, subcutaneously, or transdermally. The compositions may be administered by any convenient route, for example by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents. Administration can be systemic or local.

In yet another embodiment, the vaccine composition is administered in such a manner as to target mucous tissues in order to elicit an immune response at the site of immunization. For example, mucosa tissues such as gut associated lymphoid tissue (GALT) can be targeted for immunization by using oral administration of compositions which contain adjuvants with particular mucosa targeting properties. Additional mucosal tissues can also be targeted, such as nasopharyngeal lymphoid tissue (NALT) and bronchial-associated lymphoid tissue (BALT) (Langermann, 1996, Seminars in Gast. Dis., 7:12-18); Wizemann et al., 1999, Emerging Inf. Dis., 5:395-403; Service, 1994, Science, 265:1522-1524).

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering a an antibody of the invention or fragment thereof, care must be taken to use materials to which the FimH compositions does not absorb.

In another embodiment, the composition can be delivered in a vesicle, in particular a liposome (Langer, 1990, Science 249:1527-1533); Treat et al., 1989, in Liposomes in the Therapy of Infectious Disease and Cancer, Lopez-Berestein and Fidler (eds.), Liss, N.Y., pp. 353-365; Lopez-Berestein, ibid., pp. 317-327; see generally ibid.).

In yet another embodiment, the composition can be delivered in a controlled release system. In one embodiment, a pump may be used (Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used (e.g., Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, N.Y.; Ranger and Peppas, 1983, J. Macromol. Sci. Rev. Macromol. Chem. 23:61; Levy et al., 1985, Science 228:190; During et al., 1989, Ann. Neurol. 25:35 1; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. In yet another embodiment, a controlled release system can be placed in proximity of the therapeutic target, e.g., the urogenital tract thus requiring only a fraction of the systemic dose (e.g., Goodson, 1984, in Medical Applications of Controlled Release, supra, vol. 2, pp. 115-138).

Other controlled release systems are discussed in the review by Langer (1990, Science 249:1527-1533).

In a specific embodiment where the composition of the invention is a nucleic acid encoding a mutant polypeptide, a mutant polypeptide complex or a fragments thereof, the nucleic acid can be administered in vivo to promote expression of its encoded mutant polypeptide compositions, by constructing it as part of an appropriate nucleic acid expression vector and administering it so that it becomes intracellular, e.g., by use of a retroviral vector (U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (e.g., Joliot et al., 1991, Proc. Natl. Acad. Sci. USA 88:1864-1868), etc. Alternatively, a nucleic acid can be introduced intra-cellularly and incorporated within host cell DNA for expression by homologous recombination.

Accordingly, also provided by the invention is a method for vaccinating a primate against urogenital tract infection, which method comprises administering to the primate a purified nucleic acid containing a nucleotide sequence encoding a mutant peptide or peptide complex comprising a mutant type 1 pilin polypeptide associated with a bacterium that causes a urogenital tract infection, said nucleic acid being administered in an amount effective to produce immunoglobulin molecules that specifically bind the type 1 pilin attachment domain. Pharmaceutical compositions containing nucleic acids comprising nucleotide sequences encoding bacterial adhesin proteins, or fragments or complexes thereof, are also provided.

The dosage of the pharmaceutical formulation can be determined readily by the skilled artisan, for example, by first identifying doses effective to elecit a prophylactic or therapeutic immune response, e.g., by measuring the serum titer of vaccine specific immunoglobulins or by measuring the inhibitory ratio of serum samples, or urine samples, or mucosal secretions. In particular, doses that result in serum endpoint titers of at least 1:800, at least 1:1600, or at least 1:3200 and/or, which have at least 50% binding inhibition of E. coli to bladder cells, upon sample dilutions of at least 1:50, at least 1:100, at least 1:200, at least 1:400, at least 1:800, at least 1: 1600, or at least 1:3200, and most preferably at least 1:1600, or have detectable specific and, preferably inhibitory immunoglobulins in urine or mucosal secretions, as taught in Section 5.3.3, in an animal model, such as a Cynomolgus monkey, before identifying the optimal dosage in humans.

In preferred embodiments, a dose of the purified mutant FimCH complex of 1 μg, 5 μg, 10 μg, 20 μg, 30 μg, 50 μg, 75 μg, 100 μg, 123 μg, 150 μg, or 200 μg, or preferably 25 μg is administered. In other embodiments, the dosage is in the range of 0.25 μg to 1 μg, 1 μg to 5 μg, 1 μg to 10 μg, 1 μg to 20 μg, 1 μg to 50 μg, 1 μg to 75 μg, 1 μg to 100 μg, 1 μg to 150 μg, 1 μg to 200 μg. 5 μg to 10 μg, 10 μg to 15 μg, 10 μg to 20 μg, 15 μg to 25 μg, 20 μg to 30 μg, 30 μg to 50 μg, 25 μg to 75 μg, 50 μg to 100 μg, 75 μg to 125 μg, 50 μg to 125 μg, 50 μg to 200 μg, or 100 μg to 200 μg. For pediatric uses, a fractional dose of the pharmaceutical composition may be administered. For adult patients or patients with persistent infections, larger doses may also be used.

Vaccines of the invention may also be administered on a dosage schedule, for example, an initial administration of the vaccine composition with subsequent booster administrations. In particular embodiments, a second dose of the pharmaceutical composition is administered anywhere from two weeks to one year, preferably from one to six months, after the initial administration. Additionally, a third dose may be administered after the second dose and from three months to two years, or even longer, preferably 4 to 6 months, or 6 months to one year after the initial administration. The third dose may be optionally administered when no or low levels of specific immunoglobulins are detected in the serum and/or urine or mucosal secretions of the subject after the second dose. In a preferred embodiment, a second dose is administered approximately one month after the first administration and a third dose is administered approximately six months after the first administration. In another preferred embodiment, the second dose is administered six months after the first administration.

5.3.3 Determination of Vaccine Efficacy

Immunopotency of the pharmaceutical formulations can be determined by monitoring the immune response of a subject following immunization with a mutant protein composition, in particular the generation of immunoglobulins, particularly IgGs, which are detectable in the urine or mucosal secretions of the subject. Generation of a humoral response may be taken as an indication of a generalized immune response, other components of which, particularly cell-mediated immunity, may be important for protection against certain disorders. The disorder is UTI in a preferred embodiment. Vaccine efficacy for other mutant proteins for other indications may be determined by analogous methods using skill in the art.

Subjects can include any primate including Cynomolgus monkeys, chimpanzees and human subjects in well controlled clinical settings. In addition, bacteria causing UTI can be used to induce infection in primates experimentally. However, since many primates are a protected species, the antibody response to a vaccine of the invention can first be studied in a number of smaller, less expensive animals, with the goal of finding one or two best candidate viruses or best combinations of viruses to use in primate efficacy studies. As one example, UTI vaccines of the invention may be tested first in mice for the ability to induce an antibody response to mutant bacterial adhesin polypeptides or polypeptide complexes and to protect against bacterial challenge.

The methods of introduction of the vaccine in the test subjects may include oral, intradermal, intramuscular, intravenous, subcutaneous, intranasal or any other standard routes of immunization.

The immune response of the test subjects can be analyzed by various approaches such as: the reactivity of the resultant immune serum or urine, or mucosal secretions to E. coli pilus, as assayed by known techniques, e.g., enzyme linked immunosorbent assay (ELISA), immunoblots, radio-immunoprecipitations, etc.; or protection from UTI infections and/or attenuation of UTI symptoms in immunized hosts, for example, but not limited to, cystitis; or inhibition of binding of E. coli to cell surface residues, particularly mannose residues.

Urine and mucosa samples may be taken from the test subject every one or two weeks, and serum analyzed for inhibitory antibodies to E. coli Type 1 pilus using, e.g., a functional test for inhibitory activity such as measured by the ability to block binding of type 1 piliated bacteria (E. coli strain NU14) to transformed human bladder 182 cell line. The presence of antibodies specific for that particular mutant FimH may be assayed by ELISA using the mutant FimCH for capture protein.

Cynomolgus monkeys (Macaca fascicularis may be used to test for immunogenicity of FimH vaccine formulations of the invention. In a specific embodiment, monkeys each receive intramuscularly approximately 100 μg or other appropriate dose of the mutant adhesin in adjuvant. A control Cynomolgus monkey receives adjuvant alone. Blood is drawn weekly for 12 weeks, and serum is analyzed for functionally inhibitory antibodies to the adhesin. Urine and vaginal samples are taken to assess, by ELISA, or other antibody detection tests, particularly IgG secretion.

Furthermore, the antibodies that are produced in response to the vaccine can be assessed for functional activity, e.g., binding to the adhesin or inhibiting binding of type 1 pilin bacteria to urogenital tract cells.

A non-limiting example of a binding inhibition assay is as follows. Type 1 piliated NU14 E. coli are directly labeled with fluorescein isothiocyanate (FITC) and incubated with J82 bladder cells at a ratio of 250 bacteria/cell in the presence of preimmune or immunized serum and incubated for 30 minutes at 37° C. After multiple washes, samples are assayed by flow cytometry, and percent inhibition of bacterial binding to the cells is determined. The samples, such as serum samples, urine samples or vaginal wash samples, are diluted at 1:2, 1:4, 1:8, up to 1:3200 or more, and compared relative to preimmune samples from each subject, in order to identify an endpoint dilution when the binding inhibition is equal to or less than 50%. The binding ratio is defined as the ratio of the number of bacteria or the mean channel fluorescent (MCF) value which correlates with the number of bacteria (e.g. NU14) bound to a cell (e.g., J82) in the presence of a diluted sample from an immunized subject, relative to the number of bacteria which bind a cell in the presence of preimmune sample from a non-immunized subject.

Another non-limiting example of a binding inhibition assay is as follows. Briefly, Immulon-4 plates (Dynex Technologies, Inc., Chantilly, Va.) are coated with 2.5 μg/ml (100 ml/well) of tri-mannose-BSA (V-Labs, Covington, La.). Type 1-piliated NU14 E. coli are added to each well, incubated at 37° C. for 1 hour and after extensive washing, bound bacteria are detected with a 1:400 dilution of an anti-E. coli-HRP conjugated antibody (Biodesign, Kennebunk, Me.). OD₄₀₅ readings of these samples establish the full signal values (FSV) for binding to trimannose (approximately 2.0). Additional samples are run in the presence of 1:50 dilutions of serum to assess inhibition, where percent inhibition equals the FSV—the sample value/FSV×100. All samples are run in triplicate.

5.4 Pharmaceutical Formulations and Administration of Antibodies

The present invention is directed to antibody-based therapies which involve administering antibodies of the invention or fragments thereof to a mammal, preferably a human, for preventing, treating, or ameliorating symptoms associated with an infection. Prophylactic and therapeutic compounds of the invention include, but are not limited to, antibodies of the invention (including fragments, analogs and derivatives thereof as described herein) and nucleic acids encoding antibodies of the invention (including fragments, analogs and derivatives thereof and anti-idiotypic antibodies as described herein). Antibodies of the invention or fragments thereof may be provided in pharmaceutically acceptable compositions as known in the art or as described herein.

Antibodies of the present invention or fragments thereof that function as inhibitors of infection caused by a pathogen can be administered to a mammal, preferably a human, to treat, prevent or ameliorate one or more symptoms associated with infection. For example, antibodies or fragments thereof which disrupt or prevent the interaction between an antigen and its binding partner (e.g., host cell receptor) may be administered to a mammal, preferably a human, to treat, prevent or ameliorate one or more symptoms associated with a infection.

It is preferred to use high affinity and/or potent in vivo inhibiting antibodies and/or neutralizing antibodies that immunospecifically binds to a the pathogen antigen (e.g., FimH), for prevention of infection and therapy for infection. It is also preferred to use polynucleotides encoding high affinity and/or potent in vivo inhibiting antibodies and/or neutralizing antibodies that immunospecifically bind to the pathogen antigen.

In a specific embodiment, an antibody of the present invention or fragment thereof inhibits or decreases the pathogen's ability to infect a host by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to pathogen infection in absence of said antibodies or antibody fragments. In another embodiment, a combination of antibodies, a combination of antibody fragments, or a combination of antibodies and antibody fragments is used in the methods of the present invention. In a further embodiment, both the vaccines and antibodies can be used in combination to prevent, treat or manage disease or infection.

One or more antibodies of the present invention or fragments thereof that immunospecifically bind to one or more pathogen mutant antigens maybe used locally or systemically in the body as a therapeutic.

In one embodiment, a mammal, preferably a human, is administered a first dose of a therapeutic or pharmaceutical composition comprising less than 15 mg/kg, preferably less than 10 mg/kg, less than 5 mg/kg, less than 3 mg/kg, less than 1 mg/kg or less than 0.5 mg/kg of one or more antibodies of the invention or fragments thereof for the prevention of an infection in an amount effective to induce a serum titer of at least 1 μg/ml, preferably at least 2 μg/ml, at least 5 μg/ml, at least 10 μg/ml, at least 15 μg/ml, at least 20 μg/ml, or at least 25 μg/ml 20 days (preferably 25, 30, 35, 40 days) after the administration of the first dose and prior to the administration of a subsequent dose. Preferably, the serum titer of said antibodies or antibody fragments is less than 30 μg/ml 30 days after the administration of the first dose and prior to the administration of a subsequent dose.

The present invention encompasses sustained release formulations comprising one or more antibodies or fragments thereof which have increased in vivo half-lives.

5.4.1 Methods of Administration of Antibodies

The invention provides methods of treatment, prophylaxis, and amelioration of one or more symptoms associated with pathogen infection by administrating to a subject of an effective amount of antibody or fragment thereof, or pharmaceutical composition comprising an antibody of the invention or fragment thereof. In a preferred aspect, an antibody or fragment thereof is substantially purified (i.e., substantially free from substances that limit its effect or produce undesired side-effects). The subject is preferably a mammal such as non-primate (e.g., cows, pigs, horses, cats, dogs, rats etc.) and a primate (e.g., monkey such as a cynomolgous monkey and a human). In a preferred embodiment, the subject is a human. In specific embodiments, the subject is a woman. he antibodies are particularly useful in women previously infected with UTI, pregnant women and sexually active women. Finally, women previously infected with sexually transmitted diseases or otherwise at risk of UTI are recipients of the antibodies of the invention. In other embodiments, the subject is a diabetic, preferably a diabetic woman. In another embodiment, antibodies to WT FimCH can be administered to a diabetic subject.

Various delivery systems are known and can be used to administer an antibody of the invention or a fragment thereof, e.g., encapsulation in liposomes, microparticles, microcapsules, recombinant cells capable of expressing the antibody or antibody fragment, receptor-mediated endocytosis (see, e.g., Wu and Wu, 1987, J. Biol. Chem. 262:4429-4432), construction of a nucleic acid as part of a retroviral or other vector, etc. Methods of administering an antibody or fragment thereof, or pharmaceutical composition include, but are not limited to, parenteral administration (e.g., intradermal, intramuscular, intraperitoneal, intravenous and subcutaneous), epidural mucosal (e.g., intranasal, vaginal, buccal and oral routes), oral and topical. In a specific embodiment, antibodies of the present invention or fragments thereof, or pharmaceutical compositions are administered intramuscularly, intravenously, or subcutaneously. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5 985,309, 5,934,272, 5,874,064, 5,855,913, 5,290,540, and 4,880,078, and PCT Publication Nos. WO 92/19244, WO 97/32572, WO 97/44013, WO 98/31346, and WO 99/66903, each of which is incorporated herein by reference their entirety.

The invention also provides that an antibody or fragment thereof is packaged in a hermetically sealed container such as an ampoule or sachette indicating the quantity of antibody or antibody fragment. In one embodiment, the antibody or antibody fragment is supplied as a dry sterilized lyophilized powder or water free concentrate in a hermetically sealed container and can be reconstituted, e.g., with water or saline to the appropriate concentration for administration to a subject. Preferably, the antibody or antibody fragment is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, or at least 75 mg. The lyophilized antibody or antibody fragment should be stored at between 2 and 8° C. in its original container and the antibody or antibody fragment should be administered within 12 hours, preferably within 6 hours, within 5 hours, within 3 hours, or within 1 hour after being reconstituted. In an alternative embodiment, an antibody or fragment thereof is supplied in liquid form in a hermetically sealed container indicating the quantity and concentration of the antibody or antibody fragment. Preferably, the liquid form of the antibody or fragment thereof is supplied in a hermetically sealed container at least 1 mg/ml, more preferably at least 2.5 mg/ml, at least 5 mg/ml, at least 8 mg/ml, at least 10 mg/ml, at least 15 mg/kg, or at least 25 mg/ml.

In a specific embodiment, it may be desirable to administer the pharmaceutical compositions of the invention locally to the area in need of treatment; this may be achieved by, for example, and not by way of limitation, local topical administration, local infusion, by injection, or by means of an implant, said implant being of a porous, non-porous, or gelatinous material, including membranes, such as sialastic membranes, or fibers. Preferably, when administering a an antibody of the invention or fragment thereof, care must be taken to use materials to which the antibody or antibody fragment does not absorb.

In another embodiment, the composition can be delivered in a vesicle, in particular a liposome (see Langer, 1990, Science 249:1527-1533; Treat, et al., 1989, in Liposomes in the Therapy of Infectious Disease and Cancer, Lopez-Berestein and Fidler (eds.), Liss, New York, pp. 353-365; Lopez-Berestein, ibid., pp. 317-327; see generally ibid.).

In yet another embodiment, the composition can be delivered in a controlled release system. In one embodiment, a pump may be used (see Langer, supra; Sefton, 1987, CRC Crit. Ref. Biomed. Eng. 14:20; Buchwald etal., 1980, Surgery 88:507; Saudek et al., 1989, N. Engl. J. Med. 321:574). In another embodiment, polymeric materials can be used to achieve controlled release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989 J. Neurosurg. 7 1:105); U.S. Pat. No. 5,679,377; U.S. Pat. No. 5,916,597; U.S. Pat. No. 5,912,015; U.S. Pat. No. 5,989,463 ; U.S. Pat. No. 5,128,326; PCT Publication No. WO 99/15154; and PCT Publication No. WO 99/20253. In yet another embodiment, a controlled release system can be placed in proximity of the therapeutic target, i. e., the lungs thus requiring only a fraction of the systemic dose (see, e.g., Goodson, in Medical Applications of Controlled Release, supra, vol. 2, pp. 115-138 (1984)).

Other controlled release systems are discussed in the review by Langer (1990, Science 249:1527-1533).

In yet another embodiment, compositions comprising antibodies of the invention or fragments thereof are formulated for sustained release. Any technique known to one of skill in the art can be used to produce sustained release formulations comprising one or more antibodies of the invention or fragments thereof. See, e.g., U.S. Pat. No. 4,526,938, PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment where the composition of the invention is a nucleic acid encoding an antibody or antibody fragment, the nucleic acid can be administered in vivo to promote expression of its encoded antibody or antibody fragment, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox- like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

The present invention also provides pharmaceutical compositions. Such compositions comprise a prophylactically or therapeutically effective amount of an antibody or a fragment thereof, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical compositions administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of the antibody or fragment thereof, preferably in purified form together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocamne to ease pain, at the site of the injection.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

5.5 Recombinant Nucleic Acids

Nucleic acid sequences changes can be introduced by mutation thereby leading to changes in the amino acid sequence of the encoded protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential ” amino acid residue is required for biological activity. For example, amino acid residues that are not conserved or only semi-conserved among homologous of various species may be non-essential for activity and thus would be likely targets for alteration. Alternatively, amino acid residues that are conserved among the homologous of various species (e.g., murine and human) may be essential for activity and thus would not be likely targets for alteration.

Accordingly, another aspect of the invention pertains to nucleic acid molecules encoding a polypeptide of the invention that contain changes in amino acid residues. Such polypeptides differ in amino acid sequence from wild type protein. In one embodiment, the domain which interacts with the wild type protein's binding partner is mutated. For example, in the bacterial adhesin FimH, amino acid substitutions can be introduced into residues listed in Section 5.1 above.

An isolated nucleic acid molecule encoding a variant protein can be created by introducing one or more nucleotide substitutions, additions or deletions into the nucleotide sequence, such that one or more amino acid substitutions, additions or deletions are introduced into the encoded protein. Mutations can be introduced by standard techniques, such as site-directed mutagenesis and PCR-mediated mutagenesis. Briefly, PCR primers are designed that delete the trinucleotide codon of the amino acid to be changed and replace it with the trinucleotide codon of the amino acid to be included. This primer is used in the PCR amplification of DNA encoding the protein of interest. This fragment is then isolated and inserted into the full length cDNA encoding the protein of interest and expressed recombinantly. The resulting protein now includes the amino acid replacement.

Preferably, non-conservative amino acid substitutions are made at one or more amino acid residues. Non-conservative replacements are those that take place between families of amino acids that are unrelated in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur containing=cysteine and methionine. (See, for example, Biochemistry, 4th ed., Ed. by L. Stryer, W H Freeman and Co.: 1995).

Alternatively, mutations can be introduced randomly along all or part of the coding sequence, such as by saturation mutagenesis.

Mutagenesis may be performed in accordance with any of the techniques known in the art including, but not limited to, synthesizing an oligonucleotide having one or more modifications within the sequence to be modified. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered. A number of such primers introducing a variety of different mutations at one or more positions may be used to generated a library of mutants.

The technique of site-specific mutagenesis is well known in the art, as exemplified by various publications (see, e.g.,. Kunkel et al., Methods J. Enzymol., 154:367-82, 1987, which is hereby incorporated by reference in its entirety). In general, site-directed mutagenesis is performed by first obtaining a single-stranded vector or melting apart of two strands of a double stranded vector which includes within is sequence a DNA sequence which encodes the desired peptide. An oligonucleotide prime bearing the desired mutated sequence is prepared, generally synthetically. This primer is then annealed with the single-stranded vector, and subjected to DNA polymerizing enzymes such as T7 DNA polymerase, in order to complete the synthesis of the mutation-bearing strand. Thus, a heteroduplex is formed wherein one strand encodes the original non-mutated sequence and the second strand bears the desired mutation. This heteroduplex vector s then used to transform or transfect appropriate cells, such as E. coli cells, and clones are selected which include recombinant vectors bearing the mutated sequence arrangement. As will be appreciated, the technique typically employs a phage vector which exists in both a single stranded and double stranded form. Typical vectors useful in site-directed mutagenesis include vectors such as the M13 phage. These phage are readily commercially available and their use is generally well known to those skilled in the art. Double stranded plasmids are also routinely employed in site directed mutagenesis which eliminates the step of transferring the gene of interest from a plasmid to a phage.

Alternatively, the use of PCR™ with commercially available thermostable enzymes such as Taq DNA polymerase may be used to incorporate a mutagenic oligonucleotide primer into an amplified DNA fragment that can then be cloned into an appropriate cloning or expression vector. See, e.g., Tomic et al., 1987, Nucleic Acids Res., 18:1656; Upender et al., 1995, BioTechniques, 18:29-30, 32, 1995, for PCR™-mediated mutagenesis procedures, which are hereby incorporated in their entireties. PCR™ employing a thermostable ligase in addition to a thermostable polymerase may also be used to incorporate a phosphorylated mutagenic oligonucleotide into an amplified DNA fragment that may then be cloned into an appropriate cloning or expression vector (see e.g., Michael, 1994, BioTechniques, 16:410-2, which is hereby incorporated by reference in its entirety).

Following mutagenesis, the encoded protein can be expressed recombinantly and the activity of the protein can be determined. Those showing desired activity can then be further characterized. In the present invention, mutant proteins which lose or have decreased biological activity (e.g., binding) are of particular interest.

5.6 Protein Expression and Purification

The mutant adhesin proteins, complexes and fragments thereof (preferably mutant FimH proteins and polypeptides) maybe produced by any method available in the art.

Those skilled in the art will readily be able to purify such proteins, fragments or complexes by routine techniques.

One problem with utilizing such proteins has been that synthesis of the polypeptide, such as FimH, results in a protein that falls short of attaining its native in vivo structure. Thus, there is a difference between the in vivo conformation of such a protein and that attained by a purified recombinant form of such protein. The reason for this difference in conformation has been determined. In general, a pilin protein, such as an adhesin like FimH, has a native conformation that is at least partly determined by the in vivo interaction of such protein with an additional protein, here a periplasmic chaperone protein called FimC. The resulting FimC-FimH (or FimCH) complex is the form that presents the native FimH conformation as seen in vivo and thus by the immune system (Choudhury et al., 1999, Science 285, 1061; Sauer et al., 1999, Science 285:1058). Consequently , the methods and compositions of the invention include such complexes where said proteins are co-expressed, or otherwise formed in a combined state, with their respective periplasmic chaperone thereby yielding the native complex normally seen in vivo by the immune system following infection by a disease causing pathogen. Accordingly, the present invention further encompasses administration of such pilin complexes, i.e., complexes of FimC with a FimH polypeptide.

FimH complexes can be readily produced by recombinant methods in such a way as to incorporate therein the sequences provided by FimC in the FimCH complex, thus yielding a native structure for FimH, which structure is immunogenic in nature. In essence, the portion of the FimC molecule that binds to FimH and directs its native conformation is engineered into the FimH structure itself, at the appropriate location, to result in a native FimH structure. This portion of the FimC molecule that binds to FimH in the FimCH complex is called a “donor strand” and the mechanism of formation of the native FimH structure using only this additional strand from FimC has been referred to as “donor strand complementation.” Thus, the FimH complexes, can be produced in their “donor complemented” form to provide highly immunogenic structures for use in therapeutically effective vaccine compositions within the present invention. Such donor strand complemented forms are disclosed in detail in U.S. application Ser. No. 09/615,846, filed Jul. 13, 2000 and PCT/US00/19066, filed Jul. 13, 2000, both entitled “Donor Strand Complemented Pilus-Based Vaccines”, each of which is hereby incorporated by reference herein in its entirety.

Accordingly, in preferred embodiments, complexes of FimH and FimC are administered in the methods of the invention. Such complexes include FimH-FimC fusion proteins and complexes, preferably, containing an equimolar ratio of FimH and FimC. Any known FimC protein can be used in such complexes. Preferably the FimC protein is from the E. coli J96 isolate and has an amino acid sequence of FIG. 1. In a more preferred embodiment, a FimCH complex containing a FimH protein and a FimC protein in equimolar amounts is administered, preferably where the FimH protein has an amino acid sequence (with one or more amino acid modifications, as discussed above) of FIG. 1 and the FimC protein has an amino acid sequence of FIG. 1. As described infra, the FimCH complexes can be expressed from the same plasmid, preferably under the control of separate promoters, and isolated from the host cell, e.g., an E. coli host cell.

Complexes comprising the E. coli chaperone FimC and a FimH variant of the invention may be formed by co-expressing a FimH variant polypeptide, whose amino acid and nucleotide sequences are known in the art (such as the FimH having the amino acid sequence of FIG. 1) along with a FimC variant polypeptide, whose amino acid and nucleotide sequences are known in the art (such as the FimC having the amino acid sequence of FIG. 1), from a recombinant cell.

In addition, the FimC-mutant FimH complexes useful in vaccines can be recovered from the periplasmic spaces of cells of the indicated strains disclosed herein. These complexes are found in relatively large amounts in recombinant E. coli strains which express the FimC protein at levels in excess of those produced in wild type strains. A suitable recombinant strain is C600/pHJ9205, in which expression of FimC has been put under control of the arabinose promoter. Those skilled in the art will recognize that other promoter sequences that can be regulated easily may also be used. Of course, such cells are readily engineered to express one or more of the FimH variant polypeptides of the invention. An extract of periplasm is obtained by exposing the bacteria to lysozyme in the presence of a hypertonic sucrose solution. FimCH complexes can also be purified using conventional protein purification methods well known in the art.

In a similar manner, FimH fragments can be recombinantly produced either by having E. coli produce the full-length FimH and then fragmenting the protein or may be isolated by mannose-binding affinity purification. Thus, only fragments of the FimH protein that retain mannose binding are isolated. Preferably, such mannose-binding fragments have a label such as a his-tag included and may be purified by methods such as nickel chromatography.

In accordance with the foregoing, FimC of E. coli is available through the American Type Culture Collection (ATCC) as accession number Z37500. A FimH protein of E. coli is available as ATCC Accession No. 1361011.

The polynucleotides encoding the mutant protein or polypeptide above may have the coding sequence fused in frame to a marker sequence which allows for purification of the polypeptides of the present invention. The marker sequence maybe, for example, a hexa-histidine tag supplied by a pQE-9 vector to provide for purification of the mature polypeptides fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein (Wilson, et al., 1984, Cell, 37:767).

The proteins and polypeptide of the invention may be recombinantly produced in an E. coli species host. Mutant FimH may likewise be produced recombinantly by producing the appropriate donor strand complemented version of FimH, wherein the amino acid sequence of FimC that interacts with mutant FimH in the FimCH complex is itself engineered at the C-terminal end of mutant FimH to provide the native conformation without the need for the remainder of the FimC molecule to be present. Additionally, mutant FimH variants may also be utilized in the form of a complex comprising isolated domains thereof, especially mannose-binding domains and fragments, which domains or fragments may be linked together, either covalently or non-covalently, utilizing linking segments, such linking segments being formed of amino acid sequences or other oligomeric structures, including simple polymer structures, to provide an overall structure exhibiting immunogenic activity.

In producing said proteins, particularly the adhesin protein recombinantly, a preferred host is a species of bacteria that can be cultured under conditions such that the usher gene (if present) is not expressed. Further preferred is a host species that is missing the usher gene or has a defective usher gene. Even further preferred is a host which is missing the pilus proteins other than the FimH protein (and may also produce the chaperone, such as FimC). When an adhesin protein or a mannose binding fragment of such adhesin protein is to be produced in the absence of its chaperone protein (or to be separated from the chaperone after production), the mutant adhesin protein (or fragment) may be permitted to become properly folded in the presence of its chaperone protein and is then separated from the chaperone protein.

The present invention also relates to vectors which include polynucleotides encoding one or more of the mutant protein or polypeptides of the present invention, host cells which are genetically engineered with vectors of the invention, including host cells containing a nucleotide sequence encoding a protein of the invention operably linked to a heterologous promoter, and the production of such mutant adhesin proteins and/or chaperone proteins by recombinant techniques in an isolated and substantially immunogenically pure form.

Host cells are genetically engineered (transduced or transformed or transfected) with the vectors comprising a polynucleotide encoding a chaperone, mutant adhesin protein, or the like, which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the polynucleotides which encode such polypeptides. The culture condition, such as temperature, pH and the like, are those previously used with the host cell selected for expression, and will be apparent to the ordinarily skilled artisan.

Vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as retrovirus, vaccinia, adenovirus, fowl pox virus, and pseudorabies. However, any other vector may be used as long as it is replicable and viable in the host.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively linked to an appropriate expression control sequence(s) (promoter) to direct mRNA synthesis. As representative examples of such promoters, there may be mentioned: LTR or SV40 promoter, the E. coli. lac or trp, the phage lambda P_{L} promoter and other promoters known to control expression of genes in prokaryotic or eukaryotic cells or their viruses. The expression vector also contains a ribosome binding site for translation initiation and a transcription terminator. The vector may also include appropriate sequences for amplifying expression.

In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in prokaryotic cell culture, e.g., E. coli.

Optimal expression of a wild type FimCH complex has been achieved using a newly constructed single vector containing the FimH and FimC genes but having the advantage that each gene is under its own separate lac promoter. Thus, one lac promoter is 5′ with respect to FimC while the second lac promoter is 5′ to the FimH gene. This plasmid was successfully constructed using the common plasmid pUC19 as a background vector (Yannish-Perron, et al., 1985, Gene, 33:103-119). This new plasmid, when used to transform the host E. coli strain BL21 (as described in Phillips, et al., 1984, J. Bacteriol. 159:283-287) and then induced using IPTG at the mid-logarithmic stage of growth, gives maximal expression of the FimCH complex in the bacterial periplasmic space. This material is then extracted and purified by methods well known in the art, including those described herein. Such a plasmid can be constructed that encodes a wild type FimC in combination with a mutant FimH.

The vector containing the appropriate DNA sequence as hereinabove described, as well as an appropriate promoter or control sequence, may be employed to transform an appropriate host to permit the host to express the proteins.

As representative examples of appropriate hosts, there may be mentioned: bacterial cells, such as E. coli, Streptomyces, Salmonella typhimurium; fungal cells, such as yeast; insect cells such as Drosophila S2 and Spodoptera Sf9; animal cells such as CHO, COS or Bowes melanoma; adenoviruses; plant cells, etc. The selection of an appropriate host is deemed to be within the scope of those skilled in the art from the teachings herein.

Constructs for production of the adhesin proteins comprise a vector, such as a plasmid or viral vector, into which a sequence of the invention has been inserted, in a forward or reverse orientation. The construct may further comprise regulatory sequences, including, for example, a promoter, operably linked to the sequence. Large numbers of suitable vectors and promoters are known to those of skill in the art, and are commercially available. The following vectors are provided by way of example. Bacterial: pQE70, pQE60, pQE-9 (Qiagen, Inc.), pbs, pD10, phagescript, psiX174, pbluescript SK, pbsks, pNH8A, pNH16a, pNH18A, pNH46A (Stratagene); ptrc99a, pKK223-3 pKK233-3, pDR540, pRIT5 (Pharmacia). Eukaryotic: pWLNEO, pSV2CAT, pOG-4, pXT1, pSG (Stratagene) pSVK3, pBPV, pMSG, pSVL (Pharmacia). However, any other plasmid or vector may be used as long as they are replicable and viable in the host.

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P_{R}, P_{L} and TRP. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

The host cell for recombinant production can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., Battey, I., Basic Methods in Molecular Biology, (1986)).

The constructs in host cells can be used in a conventional manner to produce the gene product encoded by the recombinant sequence. Alternatively, the polypeptides of the invention can be synthetically produced by conventional peptide synthesizers.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts, as well as other methods in molecular biology, are described in Sambrook, et al., 1989, Molecular Cloning: A Laboratory Manual, Second Edition, Cold Spring Harbor, N.Y.; Wu et al., Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference.

Transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Examples include the SV40 enhancer on the late side of the replication origin bp 100 to 270, a cytomegalovirus early promoter enhancer, the polyoma enhancer on the late side of the replication origin, and adenovirus enhancers.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), a-factor, acid phosphatase, or heat shock proteins, among others. The heterologous structural sequence is assembled in appropriate phase with translation initiation and termination sequences. Optionally, the heterologous sequence can encode a fusion protein including an N-terminal identification peptide imparting desired characteristics, e.g., stabilization or simplified purification of expressed recombinant product.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega Biotec, Madison, Wis., U.S.A.). These pBR322 “backbone” sections are combined with an appropriate promoter and the structural sequence to be expressed.

Following transformation of a suitable host strain and growth of the host strain to an appropriate cell density, the selected promoter is induced by appropriate means (e.g., temperature shift or chemical induction) and cells are cultured for an additional period.

Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, a french press, mechanical disruption, or use of cell lysing agents, such methods are well know to those skilled in the art.

Various mammalian cell culture systems can also be employed to express recombinant protein. Examples of mammalian expression systems include the COS-7 lines of monkey kidney fibroblasts (described by Gluzman, 1981, Cell, 23:176) and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements.

The proteins and polypeptides can be recovered and/or purified from recombinant cell cultures by well-known protein recovery and purification methods. Such methodology may include ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. In this respect, chaperones may be used in such a refolding procedure. Finally, high performance liquid chromatography (HPLC) can be employed for final purification steps.

The polypeptides that are useful as immunogens in the present invention may be a naturally purified product (if a suitable naturally occurring mutant exists), or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated or may be non-glycosylated. Particularly preferred immunogens are FimH adhesin protein or fragments thereof since FimH is highly conserved among many bacterial species (see FIG. 3). Therefore, antibodies against FimH (or its mannose-binding fragments) should bind to FimH of other bacterial species (in addition to E. coli) and vaccines against E. coli FimH (or FimH mannose-binding fragments) should give protection against other bacterial infections in addition to E. coli infections (for example, against other Enterobacteriacea infections) (see, e.g., U.S. application Ser. No. 09/615,846 and PCT application No. PCT/US00/19066, both entitled “Donor Strand Complemented Pilus-Based Vaccines” and filed Jul. 13, 2000; U.S. application Ser. No. 09/616,702, filed Jul. 14, 2000, entitled “FimH Adhesin Based Vaccines” by Hultgren et al.; and U.S. Provisional Application No. 60/216,750, filed Jul. 7, 2000, entitled “FimH Adhesin Proteins” by Langermann et al.)

Procedures for the isolation of a periplasmic chaperone protein complexed with an adhesin protein are known in the art, as an example see Jones et al., (1993, Proc. Natl. Acad. Sci. USA 90:8397-8401). Further, the individually expressed adhesin proteins may be isolated by recombinant expression/isolation methods that are well-known in the art. Typical examples for such isolation may utilize an antibody to the protein or to a His tag or cleavable leader or tail that is expressing as part of the protein structure.

The FimCH polypeptides useful in forming the vaccine compositions of the present invention may conveniently be cloned using various cloning systems. The FimCH complex described therein is composed of a 52 kDa complex composed of two proteins: FimC (22.8 kDa) and FimH (29.1 kDa) in a 1:1 equimolar ratio. The FimCH complex is expressed from a pUC-based vector (pGCA139-1-1) with two separate lac-inducible promoters driving expression of the FimC and FimH genes, respectively. The FimC and the FimH genes in the pGCA139-1-1 vector were derived from uropathogenic E. coli isolate J96 and have the nucleotide sequences of FIG. 1.

The FimCH complex is produced in the periplasm of E. coli strain BL21 and is purified from periplasmic extracts by standard chromatographic methods. The FimCH protein has been formulated in a number of different buffers compatible with its solubility profile including 20 mM HEPES (pH 7.0), PBS (pH 7.0) and sodium citrate (pH 6.0) in 0.2 M NaCl. This sodium citrate/sodium chloride formulation enhances the stability of the FimCH complex and is also compatible with commonly used diluents.

Plasmid pCGA139-1-1 was constructed as a means of producing relatively large amounts of E. coli chaperone-adhesin complex, wild type FimCH. For use in the vaccine compositions disclosed herein, the wild type FimH is replaced with a mutant FimH.

The plasmid vector, pCGA139-1-1, contains the following genetic elements: (1) an E. coli FimC chaperone gene followed by (2) the FimH adhesin gene, both from E. coli strain J96 (a urinary tract infection (UTI) isolate) each preceded by its respective native signal sequence (nss); (3) a kanamycin resistance (kan^{r} or k^{r}) marker; (4) lacl^{q} which codes for a repressor protein that binds the lac promoter unless it is induced; (5) an inactivated beta-lactamase (bla) gene; (6) pUC origin of replication (ori); and (7) two lac promoters, one preceding the FimC signal and the other preceding that of FimH.

5.6.1 Fusion Proteins

In certain embodiments, the invention provides a polypeptide which is constructed as a fusion protein (e.g., covalently bonded to a different protein). The invention provides nucleic acids encoding such fusion proteins. In certain other embodiments of this invention, the nucleic acid encoding a fusion protein of the invention is operably linked to an appropriate promoter.

Fusion proteins in which a mutant FimH protein, preferably an adhesion or FimH, or a fragment of such a protein is fused to a heterologous protein are within the scope of this invention. In addition, fusion proteins can be made with antibodies of the invention or fragments thereof. Such proteins and peptides can be designed, for example, on the basis of the nucleotide sequences disclosed herein and/or on the basis of the amino acid sequences disclosed herein. Fusion proteins include, but are not limited to fusions to any amino acid sequence that allows the fusion protein to be anchored to the cell membrane; or fusions of the peptide to an enzyme, fluorescent protein, luminescent protein, or a flag epitope protein or peptide which provides a marker function.

In a specific embodiment, a polypeptide of the invention (or a nucleic acid encoding the polypeptide of the invention) is constructed as a chimeric or fusion protein. The polypeptide of the invention is joined at its amino- or carboxy-terminus via a peptide bond to an amino acid sequence of a different protein. In specific embodiments, the amino acid sequence of the different protein is at least 6, 10, 20 or 30 continuous amino acids of the different proteins or a portion of the heterologous protein that is functionally active. In specific embodiments, the amino acid sequence of the different protein is at least 50, 75, 100, or 150 continuous amino acids of the different proteins or a portion of the different protein that is functionally active. In one embodiment, such a chimeric protein is produced by recombinant expression of a nucleic acid encoding a polypeptide of the invention joined in-frame to a coding sequence for a different protein (e.g., such as a heparin binding domain). Such a chimeric product can be made by ligating the appropriate nucleic acid sequences encoding the desired amino acid sequences to each other by methods known in the art, in the proper coding frame, and expressing the chimeric product into the expression vehicle of choice by methods commonly known in the art.

Chimeric nucleic acids comprising portions of a nucleic acid encoding a polypeptide of the invention fused to any heterologous protein-encoding sequences may be constructed. In a specific embodiment, the fusion protein comprises an affinity tag such as a hexahistidine tag, or other affinity tag that may be used in purification, isolation, identification, or assay of expression. In another specific embodiment, the fusion protein comprises a protease cleavage site such as a metal protease or serine cleavage site.

Construction of fusion proteins for expression in bacteria or eukaryotic systems are well known in the art and such methods are within the scope of the invention.

Any fusion protein may be readily purified by utilizing an antibody specific for the fusion protein being expressed. For example, a system described by Janknecht et al. (1991, Proc. Natl. Acad. Sci. USA 88:8972-8976) allows for the ready purification of non-denatured fusion proteins expressed in human cell lines. In this system, the nucleic acid of interest is subcloned into a vaccinia recombination plasmid such that the open reading frame is translationally fused to an amino-terminal tag consisting of six histidine residues. Extracts from cells infected with recombinant vaccinia virus are loaded onto Ni²⁺·triloacetic acid-agarose columns and histidine-tagged proteins are selectively eluted with imidazole-containing buffers.

5.7 Antibodies Generated by the Vaccines of the Invention

Antibodies generated against mutant proteins of the invention by immunization with the vaccines formulations of the present invention also have potential uses in diagnostic assays, passive immunotherapy, and generation of antidiotypic antibodies.

Techniques described for the production of single chain antibodies (U.S. Pat. No. 4,946,778) can be adapted to produce single chain antibodies to immunogenic mutant polypeptide products of this invention. Also, transgenic mice may be used to express humanized antibodies to immunogenic mutant polypeptide products of this invention.

The vaccine formulations of the present invention can also be used to produce antibodies for use in passive immunotherapy, in which short-term protection of a host is achieved by the administration of pre-formed antibody directed against a heterologous organism.

More particularly, an isolated mutant polypeptide of the invention, or a fragment thereof, can be used as an immunogen to generate antibodies using standard techniques for polyclonal and monoclonal antibody preparation. The full-length mutant polypeptide or protein can be used or, alternatively, the invention provides antigenic peptide fragments for use as immunogens. The antigenic peptide of a mutant protein of the invention comprises at least 8 (preferably 10, 15, 20, or 30) amino acid residues, and encompasses an epitope of the mutant protein such that an antibody raised against the peptide forms a specific immune complex with the protein.

Preferred epitopes encompassed by an antigenic mutant protein are regions that are located on the surface of the protein, e.g., hydrophilic regions. In certain embodiments, the nucleic acid molecules of the invention are present as part of nucleic acid molecules comprising nucleotide sequences that contain or encode heterologous (e.g., vector, expression vector, or fusion protein) sequences. These nucleotides can then be used to express proteins which can be used as immunogens to generate an immune response, or more particularly, to generate polyclonal or monoclonal antibodies specific to the expressed protein.

An immunogen typically is used to prepare antibodies by immunizing a suitable subject, (e.g., rabbit, goat, mouse or other mammal). An appropriate immunogenic preparation can contain, for example, recombinantly expressed or chemically synthesized mutant polypeptide. The preparation can further include an adjuvant, such as Freund's complete or incomplete adjuvant, or similar immunostimulatory agent.

Accordingly, another aspect of the invention pertains to antibodies directed against a polypeptide of the invention. The term “antibody” as used herein refers to immunoglobulin molecules and immunologically active portions of immunoglobulin molecules, i. e., molecules that contain an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin.

The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared by immunizing a suitable subject with a mutant polypeptide of the invention as an immunogen. Preferred polyclonal antibody compositions are ones that have been selected for antibodies directed against a polypeptide or polypeptides of the invention. Particularly preferred polyclonal antibody preparations are ones that contain only antibodies directed against a polypeptide or polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. Alternatively, antibodies specific for a protein or polypeptide of the invention can be selected for (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those on the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

At an appropriate time after immunization, e.g, when the specific antibody titers are highest, antibody-producing cells can be obtained from the subject and used to prepare monoclonal antibodies by standard techniques, such as the hybridoma technique (originally described by Kohler and Milstein, 1975, Nature 256:495-497), the human B cell hybridoma technique (Kozbor et al, 1983, Immunol. Today 4:72), the BBV-hybridoma technique (Cole et al., 1985, Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay,

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia , Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, BioTechnology 9:1370-1372; Hay et al., 1992, Hum. Antibod. Hybridomas 3:81-85; Huse et al., 1989, Science 246:1275-1281; Griffiths et al., 1993, EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine MAB and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g, Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01 [symbol] 533; U.S. Pat. No. 4,816,567; European Patent Application 125,023; Better et al., 1988, Science 240:1041-1043; Liu et al., 1987, Proc. Natl. Acad. Sci. USA 84: 3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Pat. No. 5,225,539; Jones et al., 1986, Nature 321:552-525; Verhoeyan et al., 1988, Science 239:1534; and Beidler et al., 1988, J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., 1994, BioTechnology 12:899-903).

An antibody directed against a polypeptide of the invention can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In addition, gene sequences and gene products of the invention, including peptide fragments, as well as specific antibodies thereto, can be used for construction of fusion proteins to facilitate recovery, detection, or localization of another protein of interest.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells, and in particular, prokaryotic cells.

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), interleukin-10 (“IL-10”), interleukin-12 (“IL-12”), interferon-γ (“IFN-γ”), interferon-α(“IFN-α”), or other immune factors or growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev., 62:119-58.

An antibody with or without a therapeutic moiety conjugated to it can be used as a therapeutic that is passively administered alone or in combination with chemotherapeutic agents.

Alternatively, an antibody of the invention can be conjugated to a second antibody to form an “antibody heteroconjugate” as described by Segal in U.S. Pat. No. 4,676,980 or alternatively, the antibodies can be conjugated to form an “antibody heteropolymer” as described in Taylor et al., in U.S. Pat. Nos. 5,470, [symbol] 70 and 5,487,890.

An antibody with or without a therapeutic moiety conjugated to it can be used as a therapeutic that is administered alone or in combination with cytotoxic factor(s) and/or cytokine(s).

In yet a further aspect, the invention provides substantially purified antibodies or fragments thereof, including human or non-human antibodies or fragments thereof, which antibodies or fragments specifically bind to a polypeptide of the invention. In various embodiments, the substantially purified antibodies of the invention, or fragments thereof, can be human, non-human, chimeric and/or humanized antibodies.

In another aspect, the invention provides non-human antibodies or fragments thereof. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof. The monoclonal antibodies can be human, humanized, chimeric and/or non-human antibodies.

Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides a kit containing an antibody of the invention conjugated to a detectable substance, and instructions for use. Still another aspect of the invention is a pharmaceutical composition comprising an antibody of the invention and a pharmaceutically acceptable carrier. In preferred embodiments, the pharmaceutical composition contains an antibody of the invention, a therapeutic moiety, and a pharmaceutically acceptable carrier.

After immunization, a sample is collected from the mammal that contains an antibody that specifically recognizes the immunogen. Preferably, the polypeptide is recombinantly produced using a non-human host cell. Optionally, the antibodies can be further purified from the sample using techniques well known to those of skill in the art. The method can further comprise producing a monoclonal antibody-producing cell from the cells of the mammal. Optionally, antibodies are collected from the antibody-producing cell.

5.8 Recombinant Methods of Producing Antibodies

The antibodies of the invention or fragments thereof can be produced by any method known in the art for the synthesis of antibodies, in particular, by chemical synthesis or preferably, by recombinant expression techniques.

The nucleotide sequence encoding an antibody of the invention can be obtained from sequencing hybridoma clone DNA. If a clone containing a nucleic acid encoding a particular antibody or an epitope-binding fragment thereof is not available, but the sequence of the antibody molecule or epitope-binding fragment there of is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from any tissue or cells expressing the antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y.; and Ausubel et al., eds., 1998, Current Protocols in Molecular Biology, John Wiley & Sons, NY, which are both incorporated by reference herein their entireties), to generate antibodies having a different amino acid sequence by, for example, introducing amino acid substitutions, deletions, and/or insertions into the epitope-binding domain regions of the antibodies and preferably, into the hinge-Fc regions of the antibodies which are involved in the interaction with the FcRn. In a preferred embodiment, antibodies having one or more modifications in amino acid residues 251-256, amino acid residues 285-290, amino acid residues 308-314, amino acid residues 382-386, and/or amino acid residues 428-436 are generated.

Recombinant expression of an antibody requires construction of an expression vector containing a nucleotide sequence that encodes the antibody. Once a nucleotide sequence encoding an antibody molecule or a heavy or light chain of an antibody, or portion thereof (preferably, but not necessarily, containing the heavy, or light chain variable region) has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding the constant region of the antibody molecule with one or more modifications in the amino acid residues involved in the interaction with the FcRn (see, e.g., PCT Publication WO 86/05807; PCT Publication WO 89/01036; U.S. Pat. No. 5,122,464; Provisional Patent Application 60/254,880, filed Dec. 12, 2000 by Johnson et al.; and Provisional Patent Application 60/289,760, filed May 9, 2001 by Johnson et al.). The nucleotide sequence encoding the heavy-chain variable region, light-chain variable region, both the heavy-chain and light-chain variable regions, an epitope-binding fragment of the heavy- and/or light-chain variable region, or one or more complementarity determining regions (CDRs) of an antibody may be cloned into such a vector for expression.

The expression vector is transferred to a host cell by conventional techniques and the transfected cells are then cultured by conventional techniques to produce an antibody having an increased affinity for the FcRn and an increased in vivo half-life. Thus, the invention includes host cells containing a polynucleotide encoding an antibody, an hinge-Fc region or fragments thereof (i.e., constant regions) having one or more modifications in amino acid residues 251-256, amino acid residues 285-290, amino acid residues 308-314, amino acid residues 382-386, and/or amino acid residues 428-436, operably linked to a heterologous promoter.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces and Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; and tobacco mosaic virus , TMV) or transformed with recombinant plasmid expression vectors (e.g., Ti plasmid) containing antibody coding sequences; and mammalian cell systems (e.g., COS, CHO, BHK, 293, 3T3, and NS0 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene, 45:101, 1986, and Cockett et al., BioTechnology, 8:2, 1990).

In bacterial systems, a number of expression vectors maybe advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lacZ coding region so that a fusion protein is produced; and pIN vectors (Inouye & Inouye, 1985, Nucleic Acids Res., 13:3101-3109; Van Heeke & Schuster, 1989, J. Biol. Chem. 24:5503-5509).

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized to express an antibody molecule of the invention. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, Proc. Natl. Acad. Sci. USA, 8 1:355-359, 1984). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bitter et al., Methods in Enzymol., 153:516-544, 1987).

In addition, a host cell strain may be chosen which modulates rates the expression of the antibody sequences, or modifies and processes the antibody in the specific fashion desired. Such modifications (e.g., glycosylation) and processing (e.g., cleavage) of protein products may be important for the function of the antibody. Different host cells have characteristic and specific mechanisms for the post-translational processing and modification of proteins and gene products. Appropriate cell lines or host systems can be chosen to ensure the correct modification and processing of the antibody expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, W138, NS0 and in particular, breast cancer cell lines such as, for example, BT483, Hs578T, HTB2, BT2O and T47D, and normal mammary gland cell line such as, for example, CRL7O3O and HsS78Bst.

For long-term, high-yield production of recombinant antibodies, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., Cell, 11:223, 1977,), hypoxanthine guanine phosphoribosyltransferase (Szybalska & Szybalski, Proc. Natl. Acad. Sci. USA, 48:202, 1992), and adenine phosphoribosyltransferase (Lowy et al., Cell, 22:8-17, 1980) genes can be employed in tk-, hgprt- or aprt- cells, respectively. Also, antimetabolite resistance can be used as the basis of selection for the following genes: dhfr, which confers resistance to methotrexate (Wigler et al., Proc. Natl. Acad. Sci. USA, 77:357, 1980 and O'Hare et al., Proc. Natl. Acad. Sci. USA, 78:1527, 1981); gpt, which confers resistance to mycophenolic acid (Mulligan & Berg, Proc. Natl. Acad. Sci. USA, 78:2072, 1981); neo, which confers resistance to the aminoglycoside G-418 (Wu and Wu, Biotherapy, 3:87-95, 1991; Tolstoshev, Ann. Rev. Pharmacol. Toxicol., 32:573-596, 1993; Mulligan, Science, 260:926-932, 1993; and Morgan and Anderson, Ann. Rev. Biochem., 62 191-217, 1993; and May, TIB TECH, 11(5):155-2 15, 1993); and hygro, which confers resistance to hygromycin (Santerre et al., Gene, 30:147, 1984). Methods commonly known in the art of recombinant DNA technology may be routinely applied to select the desired recombinant clone, and such methods are described, for example, in Ausubel et al. (eds.), 1993, Current Protocols in Molecular Biology, John Wiley & Sons, NY; Kriegler, 1990, Gene Transfer and Expression, A Laboratory Manual, Stockton Press, NY; in Chapters 12 and 13, Dracopoli et al. (eds), 1994, Current Protocols in Human Genetics, John Wiley & Sons, NY; and Colberre-Garapin et al., J. Mol. Biol., 150:1, 1981, which are incorporated by reference herein in their entireties.

The expression levels of an antibody molecule can be increased by vector amplification (for a review, see Bebbington and Hentschel, 1987, The use of vectors based on gene amplification for the expression of cloned genes in mammalian cells in DNA cloning, Vol. 3. Academic Press, New York). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol., 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature, 322:52, 1986; and Kohler, Proc. Natl. Acad. Sci. USA, 77:2 197, 1980). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A purification, and sizing column chromatography), centrifugation, differential solubility, or by any other standard techniques for the purification of proteins. Further, the antibodies of the present invention or fragments thereof may be fused to heterologous polypeptide sequences described herein or otherwise known in the art to facilitate purification.

5.8.1 Antibody Conjugates

The present invention encompasses antibodies recombinantly fused or chemically conjugated (including both covalently and non-covalently conjugations) to heterologous polypeptides (i. e., an unrelated polypeptide; or portion thereof, preferably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences. Antibodies fused or conjugated to heterologous polypeptides may also be used in in vitro immunoassays and purification methods using methods known in the art. See e.g., PCT publication Number WO 93/2 1232; EP 439,095; Naramura et al., 1994 Immunol. Lett., 39:91-99; U.S. Pat. No. 5,474,981; Gillies et al., 1992, Proc. Natl. Acad. Sci. USA, 89:1428-1432; and Fell et al., 1991, J. Immunol., 146:2446-2452, which are incorporated herein by reference in their entireties.

Antibodies can be fused to marker sequences, such as a peptide to facilitate purification. In preferred embodiments, the marker amino acid sequence is a hexa-histidine peptide, such as the tag provided in a pQE vector (QIAGEN, Inc.), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell, 37:767) and the “flag” tag (Knappik et al., 1994, BioTechniques, 17:754-761).

The present invention also encompasses antibodies conjugated to a diagnostic or therapeutic agent or any other molecule for which serum half-life is desired to be increased. The antibodies can be used diagnostically to, for example, monitor the development or progression of a disease, disorder or infection as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals, and nonradioactive paramagnetic metal ions. The detectable substance may be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

An antibody may be conjugated to a therapeutic moiety such as a cytotoxin (e.g., a cytostatic or cytocidal agent), a therapeutic agent or a radioactive element (e.g., alpha-emitters, gamma-emitters, etc.). Cytotoxins or cytotoxic agents include any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, an antibody may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon (IFN-α), β-initerferon (IFN-β), nerve growth factor (NGF), platelet derived growth factor (PDGF), tissue plasminogen activator (TPA), an apoptotic agent (e.g., TNF-α, TNF-β, AIM I as disclosed in PCT Publication No. WO 97/33899), AIM II (see, PCT Publication No. WO 97/34911), Fas Ligand (Takahashi et al., 1994, J. Iminunol., 6:1567-1574), and VEGI (PCT Publication No. WO 99/23105), a thrombotic agent or an anti-angiogenic agent (e.g., angiostatin or endostatin); or a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”), or a growth factor (e.g., growth hormone (“GH”)).

Techniques for conjugating such therapeutic moieties to antibodies are well known; see, e.g., Arnon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Hellstrom et al., “Antibodies For Drug Delivery”, in Controlled Drug Delivery (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in Monoclonal Antibodies '84: Biological And Clinical Applications, Pinchera et al. (eds.), 1985, pp. 475-506); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.),1985, pp. 303-16, Academic Press; and Thorpe et al., 1982, Immunol. Rev., 62:119-58.

An antibody or fragment thereof, with or without a therapeutic moiety conjugated to it, administered alone or in combination with cytotoxic factor(s) and/or cytokine(s) can be used as a therapeutic.

Alternatively, an antibody can be conjugated to a second antibody to form an antibody heteroconjugate as described by Segal in U.S. Pat. No. 4,676,980, which is incorporated herein by reference in its entirety.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

5.9 Crystal Structure

5.9.1 Crystalline FimCH

In another aspect, the present invention provides co-crystals of FimCH complexes with a mannose sugar, the crystal structures derived therefrom and methods of their use.

In the co-crystals, the mannose sugar can be any mannose sugar including, for example, mannopentanose, methyl-alpha-D-mannopyranoside, alpha-D-mannopyranoside, mannotriose, an oligomannoside, a dimannoside, etc.

The crystals from which the atomic structure coordinates of the invention may be obtained include native crystals and heavy-atom derivative crystals. Native crystals generally comprise substantially pure polypeptides corresponding to FimCH in crystalline form.

It is to be understood that the crystalline FimCH from which the atomic structure coordinates of the invention can be obtained is not limited to wild-type FimCH. Indeed, the crystals may comprise mutants of wild-type FimCH. Mutants of wild-type FimCH are obtained by replacing at least one amino acid residue in the sequence of the wild-type FimCH with a different amino acid residue, or by adding or deleting one or more amino acid residues within the wild-type sequence and/or at the − and/or C-terminus of the wild-type FimCH.

The types of mutants contemplated by this invention include conservative mutants, non-conservative mutants, deletion mutants, truncated mutants, extended mutants, methionine mutants, selenomethionine mutants, cysteine mutants and selenocysteine mutants. A mutant may have, but need not have, FimCH activity. Methionine, selenomethione, cysteine, and selenocysteine mutants are particularly useful for producing heavy-atom derivative crystals, as described in detail, below.

It will be recognized by one of skill in the art that the types of mutants contemplated herein are not mutually exclusive; that is, for example, a polypeptide having a conservative mutation in one amino acid may in addition have several Leu or Ile to Met mutations.

The amino acid residue Cys (C) is unusual in that it can form disulfide bridges with other Cys (C) residues or other sulfhydryl-containing amino acids (“cysteine-like amino acids”). The ability of Cys (C) residues and other cysteine-like amino acids to exist in a polypeptide in either the reduced free —SH or oxidized disulfide-bridged form affects whether Cys (C) residues contribute net hydrophobic or hydrophilic character to a polypeptide. While Cys (C) exhibits a hydrophobicity of 0.29 according to the consensus scale of Eisenberg et al. (1984, J. Mol. Biol. 179:125-142.), it is to be understood that for purposes of the present invention Cys (C) is categorized as a polar hydrophilic amino acid, notwithstanding the general classifications defined above. Preferably, Cys residues that are known to participate in disulfide bridges are not substituted or are conservatively substituted with other cysteine-like amino acids so that the residue can participate in a disulfide bridge.

Typical cysteine-like residues include, for example, Pen, hCys, etc. Substitutions for Cys residues that interfere with crystallization are discussed infra.

While in most instances the amino acids of FimCH will be substituted with genetically-encoded amino acids, in certain circumstances mutants may include genetically non-encoded amino acids. For example, non-encoded derivatives of certain encoded amino acids, such as SeMet and/or SeCys, may be incorporated into the polypeptide chain using biological expression systems (such SeMet and SeCys mutants are described in more detail, infra).

Alternatively, in instances where the mutant will be prepared in whole or in part by chemical synthesis, virtually any non-encoded amino acids may be used, ranging from D-isomers of the genetically encoded amino acids to non-encoded naturally-occurring natural and synthetic amino acids.

Conservative amino acid substitutions for many of the commonly known non-genetically encoded amino acids are well known in the art. Conservative substitutions for other non-encoded amino acids can be determined based on their physical properties as compared to the properties of the genetically encoded amino acids.

In some instances, it may be particularly advantageous or convenient to substitute, delete from and/or add amino acid residues to FimCH in order to provide convenient cloning sites in cDNA encoding the polypeptide, to aid in purification of the polypeptide, etc. Such substitutions, deletions and/or additions that do not substantially alter the three dimensional structure of the native FimCH will be apparent to those having skills in the art. These substitutions, deletions and/or additions include, but are not limited to, His tags, intein-containing self-cleaving tags, maltose binding protein fusions, glutathione S-transferase protein fusions, antibody fusions, green fluorescent protein fusions, signal peptide fusions, biotin accepting peptide fusions, and the like.

Mutations may also be introduced into a polypeptide sequence where there are residues, e.g., cysteine residues, that interfere with crystallization. Such cysteine residues can be substituted with an appropriate amino acid that does not readily form covalent bonds with other amino acid residues under crystallization conditions, e.g., by substituting the cysteine with Ala, Ser or Gly. Any cysteine located in a non-helical or non-β-stranded segment, based on secondary structure assignments, are good candidates for replacement.

It should be noted that the mutants contemplated herein need not exhibit FimCH activity. Indeed, amino acid substitutions, additions or deletions that interfere with the activity of FimCH are specifically contemplated by the invention. Such crystalline polypeptides, or the atomic structure coordinates obtained therefrom, can be used to provide phase information to aid the determination of the three-dimensional X-ray structures of other related or non-related crystalline polypeptides.

The heavy-atom derivative crystals from which the atomic structure coordinates of the invention are obtained generally comprise a crystalline FimCH polypeptide in association with one or more heavy metal atoms. The polypeptide may correspond to a wild-type or a mutant FimCH, which may optionally be in co-complex with one or more molecules, as previously described. There are two types of heavy-atom derivatives of polypeptides: heavy-atom derivatives resulting from exposure of the protein to a heavy metal in solution, wherein crystals are grown in medium comprising the heavy metal, or in crystalline form, wherein the heavy metal diffuses into the crystal, and heavy-atom derivatives wherein the polypeptide comprises heavy-atom containing amino acids, e.g., selenomethionine and/or selenocysteine mutants.

In practice, heavy-atom derivatives of the first type can be formed by soaking a native crystal in a solution comprising heavy metal atom salts, or organometallic compounds, e.g., lead chloride, gold thiomalate, ethylmercurithiosalicylic acid-sodium salt (thimerosal), uranyl acetate, platinum tetrachloride, osmium tetraoxide, zinc sulfate, and cobalt hexamine, which can diffuse through the crystal and bind to the crystalline polypeptide.

Heavy-atom derivatives of this type can also be formed by adding to a crystallization solution comprising the polypeptide to be crystallized an amount of a heavy metal atom salt, which may associate with the protein and be incorporated into the crystal. The location(s) of the bound heavy metal atom(s) can be determined by X-ray diffraction analysis of the crystal. This information, in turn, is used to generate the phase information needed to construct the three-dimensional structure of the protein.

Heavy-atom derivative crystals may also be prepared from polypeptides that include one or more SeMet and/or SeCys residues (SeMet and/or SeCys mutants). Such selenocysteine or selenomethionine mutants may be made from wild-type or mutant FimCH by expression of FimCH-encoding cDNAs in auxotrophic E. coli strains. Hendrickson et al., 1990, EMBO J. 9(5):1665-1672. In this method, the wild-type or mutant FimCH cDNA may be expressed in a host organism on a growth medium depleted of either natural cysteine or methionine (or both) but enriched in selenocysteine or selenomethionine (or both). Alternatively, selenocysteine or selenomethionine mutants may be made using nonauxotrophic E. coli strains, e.g., by inhibiting methionine biosynthesis in these strains with high concentrations of Ile, Lys, Phe, Leu, Val or Thr and then providing selenomethionine in the medium (Doublié, 1997, Methods in Enzymology 276:523-530). Furthermore, selenocysteine can be selectively incorporated into polypeptides by exploiting the prokaryotic and eukaryotic mechanisms for selenocysteine incorporation into certain classes of proteins in vivo, as described in U.S. Pat. No. 5,700,660 to Leonard et al. (filed Jun. 7, 1995). One of skill in the art will recognize that selenocysteine is preferably not incorporated in place of cysteine residues that form disulfide bridges, as these may be important for maintaining the three-dimensional structure of the protein and are preferably not to be eliminated. One of skill in the art will further recognize that, in order to obtain accurate phase information, approximately one selenium atom should be incorporated for every 140 amino acid residues of the polypeptide chain. The number of selenium atoms incorporated into the polypeptide chain can be conveniently controlled by designing a Met or Cys mutant having an appropriate number of Met and/or Cys residues, as described more fully below.

In some instances, the polypeptide to be crystallized may not contain cysteine or methionine residues. Therefore, if selenomethionine and/or selenocysteine mutants are to be used to obtain heavy-atom derivative crystals, methionine and/or cysteine residues may be introduced into the polypeptide chain. Likewise, Cys residues must be introduced into the polypeptide chain if the use of a cysteine-binding heavy metal, such as mercury, is contemplated for production of a heavy-atom derivative crystal.

Such mutations are preferably introduced into the polypeptide sequence at sites that will not disturb the overall protein fold. For example, a residue that is conserved among many members of the protein family or that is thought to be involved in maintaining its activity or structural integrity, as determined by, e.g., sequence alignments, should not be mutated to a Met or Cys. In addition, conservative mutations, such as Ser to Cys, or Leu or Ile to Met, are preferably introduced. One additional consideration is that, in order for a heavy-atom derivative crystal to provide phase information for structure determination, the location of the heavy atom(s) in the crystal unit cell must be determinable and provide phase information. Therefore, a mutation is preferably not introduced into a portion of the protein that is likely to be mobile, e.g., at, or within about 1-5 residues of, the − and C-termini.

Conversely, if there are too many methionine and/or cystine residues in a polypeptide sequence, over-incorporation of the selenium-containing side chains can lead to the inability of the polypeptide to fold and/or crystallize, and may potentially lead to complications in solving the crystal structure. In this case, methionine and/or cysteine mutants are prepared by substituting one or more of these Met and/or Cys residues with another residue. The considerations for these substitutions are the same as those discussed above for mutations that introduce methionine and/or cysteine residues into the polypeptide. Specifically, the Met and/or Cys residues are preferably conservatively substituted with Leu/Ile and Ser, respectively.

As DNA encoding cysteine and methionine mutants can be used in the methods described above for obtaining SeCys and SeMet heavy-atom derivative crystals, the preferred Cys or Met mutant will have one Cys or Met residue for every 140 amino acids.

5.9.2 Crystallization of Polypeptides and Characterization of Crystals

The native, heavy-atom derivative and co-crystals from which the atomic structure coordinates of the invention are obtained can be obtained by conventional means as are well-known in the art of protein crystallography, including batch, liquid bridge, dialysis, and vapor diffusion methods (see, e.g., McPherson, 1998, ‘Crystallization of Biological Macromolecules’, Cold Spring Harbor Press, New York; McPherson, 1990, Eur. J. Biochem. 189:1-23.; Weber, 1991, Adv. Protein Chem. 41:1-36.).

Generally, native crystals are grown by dissolving substantially pure FimCH polypeptide complex in an aqueous buffer containing a precipitant at a concentration just below that necessary to precipitate the protein. Examples of precipitants include, but are not limited to, polyethylene glycol, ammonium sulfate, 2-methyl-2,4-pentanediol, sodium citrate, sodium chloride, glycerol, isopropanol, lithium sulfate, sodium acetate, sodium formate, potassium sodium tartrate, ethanol, hexanediol, ethylene glycol, dioxane, t-butanol and combinations thereof. Water is removed by controlled evaporation to produce precipitating conditions, which are maintained until crystal growth ceases.

In a preferred embodiment, native crystals are grown by vapor diffusion in sitting drops (McPherson, 1982, “Preparation and Analysis of Protein Crystals”, John Wiley, New York; McPherson, 1990, Eur. J. Biochem. 189:1-23). In this method, the polypeptide/precipitant solution is allowed to equilibrate in a closed container with a larger aqueous reservoir having a precipitant concentration optimal for producing crystals. Generally, less than about 25 μl of substantially pure polypeptide solution is mixed with an equal volume of reservoir solution, giving a precipitant concentration about half that required for crystallization. The sealed container is allowed to stand, usually for about 2-6 weeks, until crystals grow.

In one embodiment of the invention, native co-crystals of a wild type FimCH alpha-D-mannopyranoside complex from which atomic structure coordinates of the invention are obtained, can be obtained by the hanging drop method or by the sitting drop method. About 6 ul of FimCH polypeptide (4.7 mg/ml in 100 mM Tris-HCl, pH 8.2, and 7 mM alpha-D-mannopyranoside) and 6 ul reservoir solution (0.7 M ammonium sulfate and 100 mM Tris-HCl, pH 8.2) suspended over 0.5 ml reservoir solution for about 3 to 4 weeks at 20° C. provide diffraction quality crystals. The buffer solution optionally can be raised to 0.9 to 1.2 M ammonium sulfate after about two days, and the crystallization solution can be optionally microseeded with, for example, a cat whisker after one week to improve crystallization.

In another embodiment of the invention, co-crystals of a wild type FimCH Q133N methyl-alpha-D-mannopyranoside complex from which atomic structure coordinates of the invention are obtained, can be obtained by the hanging drop method or by the sitting drop method. About 6 ul of FimCH Q133N complex (4.7 mg/ml in 100 mM Tris-HCl, pH 8.2, and 10 mM methyl-alpha-D-mannopyranoside) and 6 ul reservoir solution (0.7 M ammonium sulfate and 100 mM Tris-HCl, pH 8.2) suspended over 0.5 ml reservoir solution for about 3 to 4 weeks at 20° C. provide diffraction quality crystals. The buffer solution optionally can be raised to 0.9 to 1.2 M ammonium sulfate after about two days, and the crystallization solution can be optionally microseeded with, for example, a cat whisker after one week to improve crystallization.

Of course, those having skill in the art will recognize that the above-described crystallization conditions can be varied. Such variations may be used alone or in combination, and include polypeptide solutions containing polypeptide concentrations between 0.06 to 0.12 mM, alpha-D-mannopyranoside or methyl-alpha-D-mannopyranoside concentrations between 0.5 and 30 mM, Tris-HCl concentrations between 50 mM and 100 mM, pH ranges between 7.8 and 8.6; and reservoir solutions containing ammonium sulfate concentrations between 0.6 M and 1.2 M, Tris-HCl concentrations between 50 mM and 100 mM, pH ranges between 7.8 and 8.6 and temperature ranges between 18° C. and 24° C. Other buffer solutions may be used such as Hepes buffer, so long as the desired pH range is maintained.

Heavy-atom derivative crystals can be obtained by soaking, native crystals in mother liquor containing salts of heavy metal atoms. Heavy-atom derivative crystals can also be obtained from SeMet and/or SeCys mutants, as described above for native crystals.

Mutant complexes other than those discussed above may crystallize under slightly different crystallization conditions than wild-type protein, or under very different crystallization conditions, depending on the nature of the mutation, and its location in the protein. For example, a non-conservative mutation may result in alteration of the hydrophilicity of the mutant, which may in turn make the mutant protein either more soluble or less soluble than the wild-type protein. Typically, if a protein becomes more hydrophilic as a result of a mutation, it will be more soluble than the wild-type protein in an aqueous solution and a higher precipitant concentration will be needed to cause it to crystallize. Conversely, if a protein becomes less hydrophilic as a result of a mutation, it will be less soluble in an aqueous solution and a lower precipitant concentration will be needed to cause it to crystallize. If the mutation happens to be in a region of the protein involved in crystal lattice contacts, crystallization conditions may be affected in more unpredictable ways.

Co-crystals can also be obtained by soaking a native crystal in mother liquor containing compound that binds FimCH, or by co-crystallizing FimCH in the presence of one or more binding compounds, as discussed above.

5.9.3 Characterization of Crystals

The dimensions of a unit cell of a crystal are defined by six numbers, the lengths of three unique edges, a, b, and c, and three unique angles, α, β, and γ. The type of unit cell that comprises a crystal is dependent on the values of these variables, as discussed above in Section 3.2.

When a crystal is placed in an X-ray beam, the incident X-rays interact with the electron cloud of the molecules that make up the crystal, resulting in X-ray scatter. The combination of X-ray scatter with the lattice of the crystal gives rise to nonuniformity of the scatter; areas of high intensity are called diffracted X-rays. The angle at which diffracted beams emerge from the crystal can be computed by treating diffraction as if it were reflection from sets of equivalent, parallel planes of atoms in a crystal (Bragg's Law). The most obvious sets of planes in a crystal lattice are those that are parallel to the faces of the unit cell. These and other sets of planes can be drawn through the lattice points. Each set of planes is identified by three indices, hkl. The h index gives the number of parts into which the a edge of the unit cell is cut, the k index gives the number of parts into which the b edge of the unit cell is cut, and the 1 index gives the number of parts into which the c edge of the unit cell is cut by the set of hkl planes. Thus, for example, the 235 planes cut the a edge of each unit cell into halves, the b edge of each unit cell into thirds, and the c edge of each unit cell into fifths. Planes that are parallel to the bc face of the unit cell are the 100 planes; planes that are parallel to the ac face of the unit cell are the 010 planes; and planes that are parallel to the ab face of the unit cell are the 001 planes.

When a detector is placed in the path of the diffracted X-rays, in effect cutting into the sphere of diffraction, a series of spots, or reflections, are recorded to produce a “still” diffraction pattern. Each reflection is the result of X-rays reflecting off one set of parallel planes, and is characterized by an intensity, which is related to the distribution of molecules in the unit cell, and hkl indices, which correspond to the parallel planes from which the beam producing that spot was reflected. If the crystal is rotated about an axis perpendicular to the X-ray beam, a large number of reflections is recorded on the detector, resulting in a diffraction pattern.

The unit cell dimensions and space group of a crystal can be determined from its diffraction pattern. First, the spacing of reflections is inversely proportional to the lengths of the edges of the unit cell. Therefore, if a diffraction pattern is recorded when the X-ray beam is perpendicular to a face of the unit cell, two of the unit cell dimensions may be deduced from the spacing of the reflections in the x and y directions of the detector, the crystal-to-detector distance, and the wavelength of the X-rays. Those of skill in the art will appreciate that, in order to obtain all three unit cell dimensions, the crystal must be rotated such that the X-ray beam is perpendicular to another face of the unit cell. Second, the angles of a unit cell can be determined by the angles between lines of spots on the diffraction pattern. Third, the absence of certain reflections and the repetitive nature of the diffraction pattern, which may be evident by visual inspection, indicate the internal symmetry, or space group, of the crystal. Therefore, a crystal may be characterized by its unit cell and space group, as well as by its diffraction pattern.

Once the dimensions of the unit cell are determined, the likely number of polypeptides in the asymmetric unit can be deduced from the size of the polypeptide, the density of the average protein, and the typical solvent content of a protein crystal, which is usually in the range of 30-70% of the unit cell volume (Matthews, 1968, J. Mol. Biol. 33:491-497).

The FimCH crystals of the present invention are generally characterized by a diffraction pattern. The crystals are further characterized by unit cell dimensions and space group symmetry information obtained from the diffraction patterns, as described above. The wild type FimCH alpha-D-mannopyranoside co-crystals and the FimCH Q133N methyl-alpha-D-mannopyranoside co-crystals, have a c-centered monoclinic unit cell and space group symmetry C2.

Several forms of crystalline FimCH were obtained. In the wild type FimCH alpha-D-mannopyranoside co-crystals, the unit cell has dimensions of a=138.077+/−0.2 Å, b=138.130+/−0.2 Å, c=215.352+/−0.2 Å, α=90, β=90.005, γ=90. In the FimCH Q133N methyl-alpha-D-mannopyranoside co-crystals, the unit cell has dimensions of a=138.35+/−0.2 Å, b=138.334+/−0.2 Å, c=213.212+/−0.2 Å and , β=89.983°+/−0.2°.There are likely to be 8 FimCH molecules in the asymmetric unit in both crystalline forms, related by an approximate four-fold axis.

5.9.4 Collection of Data and Determination of Structure Solutions

The diffraction pattern is related to the three-dimensional shape of the molecule by a Fourier transform. The process of determining the solution is in essence a re-focusing of the diffracted X-rays to produce a three-dimensional image of the molecule in the crystal. Since re-focusing of X-rays cannot be done with a lens at this time, it is done via mathematical operations.

The sphere of diffraction has symmetry that depends on the internal symmetry of the crystal, which means that certain orientations of the crystal will produce the same set of reflections. Thus, a crystal with high symmetry has a more repetitive diffraction pattern, and there are fewer unique reflections that need to be recorded in order to have a complete representation of the diffraction. The goal of data collection, a data set, is a set of consistently measured, indexed intensities for as many reflections as possible. A complete data set is collected if at least 80%, preferably at least 90%, most preferably at least 95% of unique reflections are recorded. In one embodiment, a complete data set is collected using one crystal. In another embodiment, a complete data set is collected using more than one crystal of the same type.

Sources of X-rays include, but are not limited to, a rotating anode X-ray generator such as a Rigaku RU-200 or a beamline at a synchrotron light source, such as the Advanced Photon Source at Argonne National Laboratory. Suitable detectors for recording diffraction patterns include, but are not limited to, X-ray sensitive film, multiwire area detectors, image plates coated with phosphorus, and CCD cameras. Typically, the detector and the X-ray beam remain stationary, so that, in order to record diffraction from different parts of the crystal's sphere of diffraction, the crystal itself is moved via an automated system of moveable circles called a goniostat.

One of the biggest problems in data collection, particularly from macromolecular crystals having a high solvent content, is the rapid degradation of the crystal in the X-ray beam. In order to slow the degradation, data is often collected from a crystal at liquid nitrogen temperatures. In order for a crystal to survive the initial exposure to liquid nitrogen, the formation of ice within the crystal must be prevented by the use of a cryoprotectant. Suitable cryoprotectants include, but are not limited to, low molecular weight polyethylene glycols, ethylene glycol, sucrose, glycerol, xylitol, and combinations thereof. Crystals may be soaked in a solution comprising the one or more cryoprotectants prior to exposure to liquid nitrogen, or the one or more cryoprotectants may be added to the crystallization solution. Data collection at liquid nitrogen temperatures may allow the collection of an entire data set from one crystal.

Once a data set is collected, the information is used to determine the three-dimensional structure of the molecule in the crystal. However, this cannot be done from a single measurement of reflection intensities because certain information, known as phase information, is lost between the three-dimensional shape of the molecule and its Fourier transform, the diffraction pattern. This phase information must be acquired by methods described below in order to perform a Fourier transform on the diffraction pattern to obtain the three-dimensional structure of the molecule in the crystal. It is the determination of phase information that in effect refocuses X-rays to produce the image of the molecule.

One method of obtaining phase information is by isomorphous replacement, in which heavy-atom derivative crystals are used. In this method, the positions of heavy atoms bound to the molecules in the heavy-atom derivative crystal are determined, and this information is then used to obtain the phase information necessary to elucidate the three-dimensional structure of a native crystal. (Blundel et al., 1976, Protein Crystallography, Academic Press).

Another method of obtaining phase information is by molecular replacement, which is a method of calculating initial phases for a new crystal of a polypeptide whose structure coordinates are unknown by orienting and positioning a polypeptide whose structure coordinates are known within the unit cell of the new crystal so as to best account for the observed diffraction pattern of the new crystal. Phases are then calculated from the oriented and positioned polypeptide and combined with observed amplitudes to provide an approximate Fourier synthesis of the structure of the molecules comprising the new crystal. (Lattman, 1985, Methods in Enzymology 115:55-77; Rossmann, 1972, “The Molecular Replacement Method,” Int. Sci. Rev. Ser. No. 13, Gordon & Breach, New York).

A third method of phase determination is multi-wavelength anomalous diffraction or MAD. In this method, X-ray diffraction data are collected at several different wavelengths from a single crystal containing at least one heavy atom with absorption edges near the energy of incoming X-ray radiation. The resonance between X-rays and electron orbitals leads to differences in X-ray scattering that permits the locations of the heavy atoms to be identified, which in turn provides phase information for a crystal of a polypeptide. A detailed discussion of MAD analysis can be found in Hendrickson, 1985, Trans. Am. Crystallogr. Assoc., 21:11; Hendrickson et al., 1990, EMBO J. 9:1665; and Hendrickson, 1991, Science 4:91.

A fourth method of determining phase information is single wavelength anomalous dispersion or SAD. In this technique, X-ray diffraction data are collected at a single wavelength from a single native or heavy-atom derivative crystal. and phase information is extracted using anomalous scattering information from atoms such as sulfur or chlorine in the native crystal or from the heavy atoms in the heavy-atom derivative crystal. The wavelength of X-rays used to collect data for this phasing technique need not be close to the absorption edge of the anomalous scatterer. A detailed discussion of SAD analysis can be found in Brodersen et al., 2000, Acta Cryst., D56:431-441.

A fifth method of determining phase information is single isomorphous replacement with anomalous scattering or SIRAS. This technique combines isomorphous replacement and anomalous scattering techniques to provide phase information for a crystal of a polypeptide. X-ray diffraction data are collected at a single wavelength, usually from a single heavy-atom derivative crystal. Phase information obtained only from the location of the heavy atoms in a single heavy-atom derivative crystal leads to an ambiguity in the phase angle, which is resolved using anomalous scattering from the heavy atoms. Phase information is therefore extracted from both the location of the heavy atoms and from anomalous scattering of the heavy atoms. A detailed discussion of SIRAS analysis can be found in North, 1965, Acta Cryst. 18:212-216; Matthews, 1966, Acta Cryst. 20:82-86.

Once phase information is obtained, it is combined with the diffraction data to produce an electron density map, an image of the electron clouds that surround the molecules in the unit cell. The higher the resolution of the data, the more distinguishable are the features of the electron density map, e.g., amino acid side chains and the positions of carbonyl oxygen atoms in the peptide backbones, because atoms that are closer together are resolvable. A model of the macromolecule is then built into the electron density map with the aid of a computer, using as a guide all available information, such as the polypeptide sequence and the established rules of molecular structure and stereochemistry. Interpreting the electron density map is a process of finding the chemically reasonable conformation that fits the map precisely.

After a model is generated, a structure is refined. Refinement is the process of minimizing the function Φ, which is the difference between observed and calculated intensity values (measured by an R-factor), and which is a function of the position, temperature factor, and occupancy of each non-hydrogen atom in the model. This usually involves alternate cycles of real space refinement, i.e., calculation of electron density maps and model building, and reciprocal space refinement, i.e., computational attempts to improve the agreement between the original intensity data and intensity data generated from each successive model. Refinement ends when the function φ converges on a minimum wherein the model fits the electron density map and is stereochemically and conformationally reasonable. During refinement, ordered solvent molecules are added to the structure.

5.9.4.1 Structures of FimCH

The present invention provides, for the first time, the high-resolution three-dimensional structures and atomic structure coordinates of crystalline FimCH bound to α-D-mannose as determined by X-ray crystallography. The specific methods used to obtain the structure coordinates are provided in the example, infra. The atomic structure coordinates of crystalline wild type FimCH-alpha-D-mannopyranoside to 2.8 Å resolution are listed in Table 14. The atomic structure coordinates of crystalline FimCH Q133N-alpha-D-mannopyranoside to 3 Å resolution are listed in Table 15.

Those having skill in the art will recognize that atomic structure coordinates as determined by X-ray crystallography are not without error. Thus, it is to be understood that any set of structure coordinates obtained for crystals of FimCH, whether native crystals, heavy-atom derivative crystals or co-crystals, that have a root mean square deviation (“r.m.s.d.”) of less than or equal to about 2 Å when superimposed, using backbone atoms (N, Cα, C and O), on the structure coordinates listed in Table 14 are considered to be identical with the structure coordinates listed in the Table when at least about 50% to 100% of the backbone atoms of FimCH are included in the superposition.

5.9.5 Structure Coordinates

The atomic structure coordinates can be used in molecular modeling and design, as described more fully below. The present invention encompasses the structure coordinates and other information, e.g., amino acid sequence, connectively tables, vector-based representations, temperature factors, etc., used to generate the three-dimensional structure of the polypeptide for use in the software programs described below and other software programs.

The invention encompasses machine readable media embedded with the three-dimensional structure of the model described herein, or with portions thereof. As used herein, “machine readable medium” refers to any medium that can be read and accessed directly by a computer or scanner. Such media include, but are not limited to: magnetic storage media, such as floppy discs, hard disc storage medium and magnetic tape; optical storage media such as optical discs or CD-ROM; electrical storage media such as RAM or ROM; and hybrids of these categories such as magnetic/optical storage media. Such media further include paper on which is recorded a representation of the atomic structure coordinates, e.g., Cartesian coordinates, that can be read by a scanning device and converted into a three-dimensional structure with an OCR.

A variety of data storage structures are available to a skilled artisan for creating a computer readable medium having recorded thereon the atomic structure coordinates of the invention or portions thereof and/or X-ray diffraction data. The choice of the data storage structure will generally be based on the means chosen to access the stored information. In addition, a variety of data processor programs and formats can be used to store the sequence and X-ray data information on a computer readable medium. Such formats include, but are not limited to, Protein Data Bank (“PDB”) format (Research Collaboratory for Structural Bioinformatics; http://www.rcsb.org/pdb/docs/format/pdbguide2.2/guide2.2_frame.html); Cambridge Crystallographic Data Centre format (http://www.ccdc.cam.ac.uk/support/csd_doc/volume3/z323.html); Structure-data (“SD”) file format (MDL Information Systems, Inc.; Dalby et al., 1992, J. Chem. Inf. Comp. Sci. 32:244-255), and line-notation, e.g., as used in SMILES (Weininger, 1988, J. Chem. Inf. Comp. Sci. 28:31-36). Methods of converting between various formats read by different computer software will be readily apparent to those of skill in the art, e.g., BABEL (v. 1.06, Walters & Stahl, ©1992, 1993, 1994; http://www.brunel.ac.uk/departments/chem/babel.htm.). All format representations of the polypeptide coordinates described herein, or portions thereof, are contemplated by the present invention. By providing computer readable medium having stored thereon the atomic coordinates of the invention, one of skill in the art can routinely access the atomic coordinates of the invention, or portions thereof, and related information for use in modeling and design programs, described in detail below.

While Cartesian coordinates are important and convenient representations of the three-dimensional structure of a polypeptide, those of skill in the art will readily recognize that other representations of the structure are also useful. Therefore, the three-dimensional structure of a polypeptide, as discussed herein, includes not only the Cartesian coordinate representation, but also all alternative representations of the three-dimensional distribution of atoms. For example, atomic coordinates may be represented as a Z-matrix, wherein a first atom of the protein is chosen, a second atom is placed at a defined distance from the first atom, a third atom is placed at a defined distance from the second atom so that it makes a defined angle with the first atom. Each subsequent atom is placed at a defined distance from a previously placed atom with a specified angle with respect to the third atom, and at a specified torsion angle with respect to a fourth atom. Atomic coordinates may also be represented as a Patterson function, wherein all interatomic vectors act e drawn and are then placed with their tails at the origin. This representation is particularly useful for locating heavy atoms in a unit cell. In addition, atomic coordinates may be represented as a series of vectors having magnitude and direction and drawn from a chosen origin to each atom in the polypeptide structure. Furthermore, the positions of atoms in a three-dimensional structure may be represented as fractions of the unit cell (fractional coordinates), or in spherical polar coordinates.

Additional information, such as thermal parameters, which measure the motion of each atom in the structure, chain identifiers, which identify the particular chain of a multi-chain protein in which an atom is located, and connectivity information, which indicates to which atoms a particular atom is bonded, is also useful for representing a three-dimensional molecular structure.

5.9.6 Uses of the Atomic Structure Coordinates

Structure information, typically in the form of the atomic structure coordinates, can be used in a variety of computational or computer-based methods to, for example, design, screen for and/or identify compounds that bind the crystallized polypeptide or a portion or fragment thereof, or to intelligently design mutants that have altered biological properties.

In one embodiment, the crystals and structure coordinates obtained therefrom are useful for identifying and/or designing compounds that bind FimC, FimH, FimCH, or a fragment thereof, as an approach towards developing new therapeutic agents. For example, a high resolution X-ray structure will often show the locations of ordered solvent molecules around the protein, and in particular at or near putative binding sites on the protein. This information can then be used to design molecules that bind these sites, the compounds synthesized and tested for binding in biological assays. Travis, 1993, Science 262:1374.

In another embodiment, the structure can be probed with a plurality of molecules to determine their ability to bind to FimC, FimH, FimCH, or a fragment thereof, at various sites. Such compounds can be used as targets or leads in medicinal chemistry efforts to identify, for example, inhibitors of potential therapeutic importance. For example, the structure coordinates can be used to identify compounds that inhibit mannose binding by FimCH. Such compounds can be used, for example, to treat or prevent urinary tract infection by a pathogen expressing FimC, FimH or FimCH.

In yet another embodiment, the structure can be used to computationally screen small molecule data bases for chemical entities or compounds thank can bind in whole, or in part, to FimC, FimH, FimCH, or a fragment thereof. In this screening, the quality of fit of such entities or compounds to the binding site may be judged either by shape complementarity or by estimated interaction energy. Meng et al., 1992, J. Comp. Chem. 13:505-524.

The design of compounds that bind to FimC, FimH, FimCH, or a fragment thereof, according to this invention generally involves consideration of two factors. First, the compound must be capable of physically and structurally associating with FimC, FimH, FimCH, or a fragment thereof. This association can be covalent or non-covalent. For example, covalent interactions may be important for designing irreversible inhibitors of a protein. Non-covalent molecular interactions important in the association of FimC, FimH, FimCH, or a fragment thereof, with its substrate include hydrogen bonding, ionic interactions and van der Waals and hydrophobic interactions. Second, the compound must be able to assume a conformation that allows it to associate with FimC, FimH, FimCH, or a fragment thereof. Although certain portions of the compound will not directly participate in this association with FimC, FimH, FimCH, or a fragment thereof, those portions may still influence the overall conformation of the molecule. This, in turn, may have a significant impact on potency. Such conformational requirements include the overall three-dimensional structure and orientation of the chemical group or compound in relation to all or a portion of the binding site, or the spacing between functional groups of a compound comprising several chemical groups that directly interact with FimC, FimH, FimCH, or a fragment thereof].

The potential inhibitory or binding effect of a chemical compound on FimC, FimH, FimCH, or a fragment thereof, may be analyzed prior to its actual synthesis and testing by the use of computer modeling techniques. If the theoretical structure of the given compound suggests insufficient interaction and association between it and FimC, FimH, FimCH, or a fragment thereof, synthesis and testing of the compound is unnecessary. However, if computer modeling indicates a strong interaction, the molecule may then be synthesized and tested for its ability to bind to FimC, FimH, FimCH, or a fragment thereof, and inhibit its activity. In this manner, synthesis of ineffective compounds may be avoided.

An inhibitory or other binding compound of FimC, FimU, FimCH, or a fragment thereof, may be computationally evaluated and designed by means of a series of steps in which chemical groups or fragments are screened and selected for their ability to associate with the individual binding pockets or other areas of FimC, FimH, FimCH, or a fragment thereof. One skilled in the art may use one of several methods to screen chemical groups or fragments for their ability to associate with FimC, FimH, FimCH, or a fragment thereof. This process may begin by visual inspection of, for example, the active site on the computer screen based on the coordinates of FimC, FimH, FimCH, or a fragment thereof. Selected fragments or chemical groups may then be positioned in a variety of orientations, or docked, within an individual binding pocket of FimC, FimH, FimCH, or a fragment thereof, as defined supra. Docking may be accomplished using software such as QUANTA and SYBYL, followed by energy minimization and molecular dynamics with standard molecular mechanics forcefields, such as CHARMM and AMBER.

Specialized computer programs may also assist in the process of selecting fragments or chemical groups. These include:

1. GRID (Goodford, 1985, J. Med. Chem. 28:849-857). GRID is available from Oxford University, Oxford, UK;

2. MCSS (Miranker & Karplus, 1991, Proteins: Structure, Function and Genetics 11:29-34). MCSS is available from Molecular Simulations, Burlington, Mass.;

3. AUTODOCK (Goodsell & Olsen, 1990, Proteins: Structure, Function, and Genetics 8:195-202). AUTODOCK is available from Scripps Research Institute, La Jolla, Calif.; and

4. DOCK (Kuntz et al., 1982, J. Mol. Biol. 161:269-288). DOCK is available from University of California, San Francisco, Calif.

Once suitable chemical groups or fragments have been selected, they can be assembled into a single compound or inhibitor. Assembly may proceed by visual inspection of the relationship of the fragments to each other in the three-dimensional image displayed on a computer screen in relation to the structure coordinates of FimC, FimH, FimCH, or a fragment thereof. This would be followed by manual model building using software such as QUANTA or SYBYL.

Useful programs to aid one of skill in the art in connecting the individual chemical groups or fragments include:

1. CAVEAT (Bartlett et al., 1989, ‘CAVEAT: A Program to Facilitate the Structure-Derived Design of Biologically Active Molecules’. In Molecular Recognition in Chemical and Biological Problems’, Special Pub., Royal Chem. Soc. 78:182-196). CAVEAT is available from the University of California, Berkeley, Calif.;

2. 3D Database systems such as MACCS-3D (MDL Information Systems, San Leandro, Calif.). This area is reviewed in Martin, 1992, J. Med. Chem. 35:2145-2154); and

3. HOOK (available from Molecular Simulations, Burlington, Mass.).

Instead of proceeding to build an inhibitor of FimC, FimH, FimCH, or a fragment thereof, in a step-wise fashion one fragment or chemical group at a time, as described above, compounds that bind may be designed as a whole or ‘de novo’ using either an empty active site or optionally including some portion(s) of a known inhibitor(s). These methods include:

1. LUDI (Bohm, 1992, J. Comp. Aid. Molec. Design 6:61-78). LUDI is available from Molecular Simulations, Inc., San Diego, Calif.;

2. LEGEND (Nishibata & Itai, 1991, Tetrahedron 47:8985). LEGEND is available from Molecular Simulations, Burlington, Mass.; and

3. LeapFrog (available from Tripos, Inc., St. Louis, Mo.).

Other molecular modeling techniques may also be emplofed in accordance with this invention. See, e.g., Cohen et al., 1990, J. Med. Chem. 33:883-894. See also, Navia & Murcko, 1992, Current Opinions in Structural Biology 2:202-210.

Once a compound has been designed or selected by the above methods, the efficiency with which that compound may bind to FimC, FimH, FimCH. or a fragment thereof, may be tested and optimized by computational evaluation. For example, a compound that has been designed or selected to function as an inhibitor of FimC, FimH, FimCH, or a fragment thereof, must also preferably occupy a volume not overlapping the volume occupied by the active site residues when the native substrate is bound. An effective inhibitor must preferably demonstrate a relatively small difference in energy between its bound and free states (i.e., it must have a small deformation energy of binding). Thus, the most efficient inhibitors should preferably be designed with a deformation energy of binding of not greater than about 10 kcal/mol, preferably, not greater than 7 kcal/mol. Inhibitors may interact with the protein in more than one conformation that is similar in overall binding energy. In those cases, the deformation energy of binding is taken to be the difference between the energy of the free compound and the average energy of the conformations observed when the inhibitor binds to the enzyme.

A compound selected or designed for binding to FimC, FimH, FimCH, or a fragment thereof, may be further computationally optimized so that in its bound state it would preferably lack repulsive electrostatic interaction with the target protein. Such non-complementary electrostatic interactions include repulsive charge-charge, dipole-dipole and charge-dipole interactions. Specifically, the sum of all electrostatic interactions between the inhibitor and the protein when the inhibitor is bound to it preferably make a neutral or favorable contribution to the enthalpy of binding.

Specific computer software is available in the art to evaluate compound deformation energy and electrostatic interaction. Examples of programs designed for such uses include: Gaussian 92, revision C (Frisch, Gaussian, Inc., Pittsburgh, Pa. (©1992); AMBER, version 4.0 (Kollman, University of California at San Francisco, ©1994); QUANTA/CHARMM (Molecular Simulations, Inc., Burlington, Mass., ©1994); and Insight II/Discover (Biosym Technologies Inc., San Diego, Calif., ©1994). These programs may be implemented, for instance, using a computer workstation, as are well-known in the art. Other hardware systems and software packages will be known to those skilled in the art.

Once a binding compound has been optimally selected or designed, as described above, substitutions may then be made in some of its atoms or chemical groups in order to improve or modify its binding properties. Generally, initial substitutions are conservative, i.e., the replacement group will have approximately the slime size, shape, hydrophobicity and charge as the original group. One of skill in the art will understand that substitutions known in the art to alter conformation should be avoided. Such altered chemical compounds may then be analyzed for efficiency of binding to FimC, FimH, FimCH, or a fragment thereof, by the same computer methods described in detail above.

Because FimC, FimH, FimCH, or a fragment thereof, may crystallize in more than one crystal form, the structure coordinates of FimC, FimH, FimCH, or a fragment thereof, are particularly useful to solve the structure of those other crystal forms of FimC, FimH, FimCH, or a fragment thereof. They may also be used to solve the structure of mutants, co-complexes, or of the crystalline form of any other protein with significant amino acid sequence homology to any functional domain of FimC, FimH or FimCH.

One method that may be employed for this purpose is molecular replacement. In this method, the unknown crystal structure, whether it is another crystal form of FimC, FimH, FimCH, or a fragment thereof, a mutant, or a co-complex, or the crystal of some other protein with significant amino acid sequence homology to any functional domain of FimC, FimH or FimCH, may be determined using phase information from the structure coordinates. This method may provide an accurate three-dimensional structure for the unknown protein in the new crystal more quickly and efficiently than attempting to determine such information ab initio. In addition, in accordance with this invention, mutants may be crystallized in co-complex with known inhibitors. The crystal structures of a series of such complexes may then be solved by molecular replacement and compared with that of wild-type FimC, FimH, FimCH, or a fragment thereof. Potential sites for modification within the various binding sites of the protein may thus be identified. This information provides an additional tool for determining the most efficient binding interactions, for example, increased hydrophobic interactions, between FimC, FimH, FimCH, or a fragment thereof, and a chemical group or compound.

If an unknown crystal form has the same space group as and similar cell dimensions to the known FimC, FimH or FimCH crystal form, then the phases derived from the known crystal form can be directly applied to the unknown crystal form, and in turn, an electron density map for the unknown crystal form can be calculated. Difference electron density maps can then be used to examine the differences between the unknown crystal form and the known crystal form. A difference electron density map is a subtraction of one electron density map, e.g., that derived from the known crystal form, from another electron density map, e.g., that derived from the unknown crystal form. Therefore, all similar features of the two electron density maps are eliminated in the subtraction and only the differences between the two structures remain. For example, if the unknown crystal form is of a co-complex, then a difference electron density map between this map and the map derived from the native, uncomplexed crystal will ideally show only the electron density of the ligand. Similarly, if amino acid side chains have different conformations in the two crystal forms, then those differences will be highlighted by peaks (positive electron density) and valleys (negative electron density) in the difference electron density map, making the differences between the two crystal forms easy to detect. However, if the space groups and/or cell dimensions of the two crystal forms are different, then this approach will not work and molecular replacement must be used in order to derive phases for the unknown crystal form.

All of the complexes referred to above may be studied using well-known X-ray diffraction techniques and may be refined versus 50 Å to 1.5 Å or greater resolution X-ray data to an R value of about 0.20 or less using computer software, such as CNS (Yale University, (c) 1992, distributed by Molecular Simulations, Inc.). See, e.g., Blundel et al., 1976, Protein Crystallography, Academic Press.; Methods in Enzymologv, vol. 114 & 115, Wyckoff et al, eds., Academic Press, 1985. This information may thus be used to optimize known classes of inhibitors, and more importantly, to design and synthesize novel classes of inhibitors.

The structure coordinates of mutants will also facilitate the identification of related proteins or enzymes analogous to FimC, FimH, FimCH, or a fragment thereof, in function, structure or both, thereby further leading to novel therapeutic modes for treating or preventing FimC, FimH or FimCH, mediated diseases.

Subsets of the atomic structure coordinates can be used in any of the above methods. Particularly useful subsets of the coordinates include, but are not limited to, coordinates of single domains, coordinates of residues lining an active site, coordinates of residues that participate in important protein-protein contacts at an interface, and Cα coordinates. For example, the coordinates of one domain of a protein that contains the active site may be used to design inhibitors that bind to that site, even though the protein is fully described by a larger set of atomic coordinates. Therefore, a set of atomic coordinates that define the entire polypeptide chain, although useful for many applications, do not necessarily need to be used for the methods described herein.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

All publications, patents and patent applications mentioned in this specification are herein incorporated by reference into the specification to the same extent as of each individual publication, patent or patent application was specifically and individually indicated to be incorporated herein by reference.

The present invention will now be further described by way of the following non-limiting examples. In applying the disclosure of these examples, it should be kept clearly in mind that other and different embodiments of the methods disclosed according to the present invention will no doubt suggest themselves to those of skill in the relevant art.

### 6. EXAMPLES

### 6.1 Example 1

### Characterization of FimH Mutants

Based on the crystal structure (FIG. 2) of vaccine quality FimCH bound to mono-mannose, the mannose-binding domain on FimH was identified. This domain was in a canyon on the surface of the protein. Furthermore, some of the specific amino acids on FimH mediating the interaction with mannose were identified. A hydrophobic ring around the mannose-binding pocket was also identified. To probe critical structural and conformational requirements of FimH, the crystal structure was used to provide several candidate residues for mutation. The serine at position 62 was mutated to an alanine and used as a control since it does not lay within the pocket or the hydrophobic ring region.

6.1.1 Expression and Isolation of FimCH mutants

Site specific mutations in FimH (see Table 7) were made according to techniques known in the art. A two-step PCR protocol as described for the mutagenesis of papD (Hung et al., 1999, Proc. Natl. Acad. Sci. USA 96:8178-83) was used. The following primers were used to amplify and introduce mutations in the first half of the FimH gene (*=coding strand; #=noncoding strand):

| | | |
|---|---|---|
| EcoRI | *5′-GGGGGGAATTCACCCGGAGGGATGATTGTA-3′ | (SEQ ID NO:5) |
| XcmI | #5′-CCAGTAGGCACCACCACATCATTATTGG-3′ | (SEQ ID NO:6) |
| F1A | *5′-CTGGTCGGTAAATGCCTGGTCAGCGGCCTGTAAAACCGCCAATGGTAC-3′ | (SEQ ID NO:7) |
| | #5′-GTACCATTGGCGGTTTTACAGGCCGCTGACCAGGCATTTACCGACCAG-3′ | (SEQ ID NO:8) |
| I13A | *5′-GCCAATGGTACCGCTATCCCTGCGGGCGGTGGCAGCGCCAATG-3′ | (SEQ ID NO:9) |
| | #5′-CATTGGCGCTGCCACCGCCCGCAGGGATAGCGGTACCATTGGC-3′ | (SEQ ID NO:10) |
| I52A | *5′-CCATAACGATTATCCGGAAACCGCGACAGACTATGTCACACTGC-3′ | (SEQ ID NO:11) |
| | *5′-GCAGTGTGACATAGTCTGTCGCGGTTTCCGGATAATCGTTATGG-3′ | (SEQ ID NO:12) |
| S62A | *5′-GCAACGAGGCGCCGCTTATGGCGG-3′ | (SEQ ID NO:13) |
| | #5′-CCGCCATAAGCGGCGCCTCGTTGC-3′ | (SEQ ID NO:14) |
| N46A | *5′-CTTTTGCCATGCTGATTATCCGGAAACC-3′ | (SEQ ID NO:15) |
| | #5′-GGTTTCCGGATAATCAGCATGGCAAAAC-3′ | (SEQ ID NO:16) |
| N46D | *5′-CTTTTGCCATGATGATTATCCGGAAACC-3′ | (SEQ ID NO:17) |
| | #5′-GGTTTCCGGATAATCATCATGGCAAAAC-3′ | (SEQ ID NO:18) |
| Y48A | *5′-GCAAATCTTTTGCCATAACGATGCGCCGGAAACCATTACAGACTATGTCACACTG-3′ | (SEQ ID NO:19) |
| | #5′-CAGTGTGACATAGTCTGTAATGGTTTCCGGCGCATCGTTATGGCAAAAGATTTGC-3′ | (SEQ ID NO:20) |
| D54A | *5′ACCATTACAGCTTATGTCACACTG-3′ | (SEQ ID NO:21) |
| | #5′-CAGTGTGACATAAGCTGTAATGGT-3′ | (SEQ ID NO:22) |
| D54N | *5′-ACCATTACAAACTATGTCACACTG-3′ | (SEQ ID NO:23) |
| | #5′-CAGTGTGACATAGTTTGTAATGGT-3′ | (SEQ ID NO:24) |
| Q133A | *5′-CTTATTTTGCGCGCTACCAACAAC-3′ | (SEQ ID NO:25) |
| | #5′-GTTGTTGGTAGCGCGCAAAATAAG-3′ | (SEQ ID NO:26) |
| Q133N | *5′-CTTATTTTGCGAAATACCAACAAC-3′ | (SEQ ID NO:27) |
| | #5′-GTTGTTGGTATTTCGCAAAATAAG-3′ | (SEQ ID NO:28) |
| Q133K | *5′-CTTATTTTGCGGAAGACCAACAAC-3′ | (SEQ ID NO:29) |
| | #5′-GTTGTTGGTCTTCCGCAAAATAAG-3′ | (SEQ ID NO:30) |
| Q133E | *5′-GCCGTGCTTATTTTGCGAGAAACCAACAACTATAACAGCGATG-3′ | (SEQ ID NO:31) |
| | #5′-CATCGCTGTTATAGTTGTTGGTTTCTCGCAAAATAAGCACGGC-3′ | (SEQ ID NO:32) |
| Q133R | *5′-GCCGTGCTTATTTTGCGACGCACCAACAACTATAACAGCGATG-3′ | (SEQ ID NO:33) |
| | #5′-CATCGCTGTTATAGTTGTTGGTGCGTCGCAAAATAAGCACGGC-3′ | (SEQ ID NO:34) |
| Q133H | *5′-GCCGTGCTTATTTTGCGACATACCAACAACTATAACAGCGATG-3′(SEQ ID NO:35) | |
| | #5′-CATCGCTGTTATAGTTGTTGGTATGTCGCAAAATAAGCACGGC-3′ | (SEQ ID NO:36) |
| N135A | *5′-GCGACAGACGGCCAACTATAACAGC-3′ | (SEQ ID NO:37) |
| | #5′-GCTGTTATAGTTGGCCGTCTGTCGC-3′ | (SEQ ID NO:38) |
| N135D | *5′-GCGACAGACCGATAACTATAACAGC-3′ | (SEQ ID NO:39) |
| | #5′-GCTGTTATAGTTSTCGGTCTGTCGC-3′ | (SEQ ID NO:40) |
| Y137A | *5′-GCGACAGACCAACAACGCGAACAGCGATGATTTCCAGTTTGTG-3′ | (SEQ ID NO:41) |
| | #5′-CACAAACTGGAAATCATCGCTGTTCGCGTTGTTGGTCTGTCGC-3′ | (SEQ ID NO:42) |
| D140A | *5′-CTATAACAGTGCAGATTTCCAG-3′ | (SEQ ID NO:43) |
| | #5′-CTGGAAATCTGCACTGTTATAG-3′ | (SEQ ID NO:44) |
| D140N | *5′-CTATAACAGCAATGATTTCCAG-3′ | (SEQ ID NO:45) |
| | #5′-CTGGAAATCATTGCTGTTATAG-3′ | (SEQ ID NO:46) |
| D140E | *5′-CTATAACAGCGAAGACTTCCAG-3′ | (SEQ ID NO:47) |
| | #5′-CTGGAAGTCTTCGCTGTTATAG-3′ | (SEQ ID NO:48) |
| F142A | *5′-GCGACAGACCAACAACTATAACAGCGATGATGCGCAGTTTGTG-3′ | (SEQ ID NO:49) |
| | #5′-CACAAACTGCGCATCATCGCTGTTATAGTTGTTGGTCTGTCGC-3′ | (SEQ ID NO:50) |

EcoR I and Xcm I restriction sites were engineered into the 5′ and 3′ primers, respectively for cloning. The PCR inserts were cloned into an EcoR1 and Xcm1 digested pHACW18 to generate a full-length FimH gene containing the desired mutations. Mutations in FimH were confirmed by sequencing. Each mutant was subcloned as, an EcoR I-BamH I full-length FimH gene into the IPTG-inducible expression vector, pMMB66 (Furste et al., 1986, Gene 48:119-31). The resulting plasmids, pHACWF1A, pHACW113A, pHACWY48A, pHACWI52A, pHACWS62A, pHACWN46A, pHACW-N46D, pHACWD54A, pHACWD54N, pHACWQ133A, pHACWQ133N, pHACWQ133K, pHACWQ 133E, pHACWQ 133R, pHACWQ133H, pHACWN135A, pHACWN135D, pHACWY137A, pHACWD140A, pHACWD140N, pHACWD140E, pHACWF142A encode FimH with point mutations changing Phe-1 to Ala; Ile-13 to Ala; Tyr-48 to Ala; Ile-52 to Ala; Ser-62 to Ala; Asn-46 to Ala or Asp; Asp-54 to Ala or Asn; Gln-133 to Ala, Asn, Lys, Glu, Arg, or His; Asn-135 to Ala or Asp; Tyr-137 to Ala; Asp140 to Ala, Asn, or Glu; and Phe-142 to Ala. Additionally, the FimH gene may be cloned into the pCGA139-1-1 vector (see Section 5.6) for expression. The wild type FimH gene from pHACW18 was also cloned into pMMB66 in the similar manner and designated as pHACW66. The original pMMB66 expression vector was used as the negative control plasmid for FimH expression. All plasmids were transformed into E. coli strains ORN103/pUT2002, AAEC185/pUT2002, C600/pHJ9205, and K12.

Wild type FimCH is a made up of an˜52 kDa complex composed of two wild type proteins; FimC (22.8 kDa) and FimH (29.1 kDa) in a 1:1 equimolar ratio. Periplasmic extracts were isolated as described (Slonim et al, 1992, EMBO J. 11:4747-56 and Jones et al., 1993, Proc. Natl. Acad. Sci. USA 90:8397-8401). Bacterial strain C600/pHJ9205 or K12 transformed with FimH expression constructs was used to produce large quantities of FimH proteins. These transformants were grown in LB in the presence of 0.1% arabinose and 0.1 mM IPTG to induce FimC and FimH expression. respectively. The protocol for the purification of FimCH complexes from bacterial periplasm has been described previously and was followed in this study (Barnhart et al., 2000, Proc. Natl. Acad. Sci. USA 97:7709-14, incorporated herein by reference). Purified FimCH complexes were dialyzed into 20 mM MES, pH 6.8 and stored at 4° C.

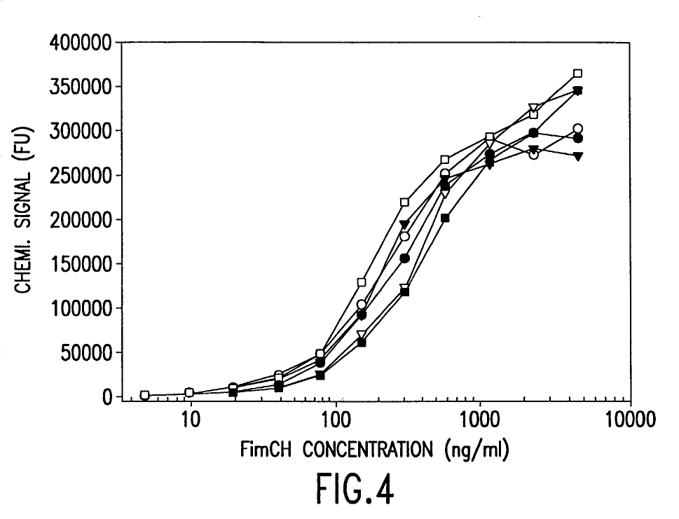

Purified recombinant FimH proteins associated with wild type FimC protein. This was assayed by ELISA using an anti-FimC antibody to detect FimCH complexes (FIG. 4). Each of the mutant proteins was expressed, associated with FimC, and localized to the periplasm (data not shown).

TABLE 7
Site Directed Mutagenesis of FimH

| residue position | wild type amino acid | engineered mutant amino acid |
|---|---|---|
| 1 | phenylalanine (F) | alanine (A) |
| 13 | isoleucine (I) | alanine (A) |
| 46 | asparagine (N) | alanine (A) |
| 46 | asparagine (N) | aspartic acid (D) |
| 48 | tyrosine (Y) | alanine (A) |
| 52 | isoleucine (I) | alanine (A) |
| 54 | aspartic acid (D) | alanine (A) |
| 54 | aspartic acid (D) | asparagine (N) |
| 62* | serine (S) | alanine (A) |
| 67 | asparagine (N) | alanine (A) |
| 67 | asparagine (N) | aspartic acid (D) |
| 75 | aspartic acid (D) | alanine (A) |
| 75 | aspartic acid (D) | asparagine (N) |
| 133 | glutamine (Q) | alanine (A) |
| 133 | glutamine (Q) | lysine (K) |
| 133 | glutamine (Q) | asparagine (N) |
| 133 | glutamine (Q) | histidine (H) |
| 133 | glutamine (Q) | arginine (R) |
| 133 | glutamine (Q) | glutamic acid (E) |
| 135 | asparagine (N) | alanine (A) |
| 135 | asparagine (N) | aspartic acid (D) |
| 135 | asparagine (N) | lysine (K) |
| 137 | tyrosine (Y) | alanine (A) |
| 140 | aspartic acid (D) | alanine (A) |
| 140 | aspartic acid (D) | asparagine (N) |
| 140 | aspartic acid (D) | glutamic acid (E) |
| 142 | phenylalanine (F) | alanine (A) |
| 154 | glutamic acid (E) | alanine (A) |
| 154 | glutamic acid (E) | asparagine (N) |
| 154 | glutamic acid (E) | lysine (K) |
| 156 | asparagine (N) | alanine (A) |
| 156 | asparagine (N) | aspartic acid (D) |
| 161 | aspartic acid (D) | alanine (A) |
| 161 | aspartic acid (D) | asparagine (N) |
| 161 | aspartic acid (D) | glutamic acid (E) |

*control reside outside of the mannose-binding pocket and hydrophobic ring regions.

6.1.2 Bacterial Surface Staining of FimH Proteins

Bacterial strain AAEC185/pUT2002 contained a FimH-null type 1 pilus operon and was complemented with either wild type or each of the mutant FimH expression plasmids. These bacteria were cultured in the same manner as the ORN103/pUT2002 transformants for optimal FimH and type 1 pili expression. Overnight cultures were diluted to the same concentration (OD₆₀₀ 1) and 1 ml of diluted bacteria was used to immunostain for FimH on the bacterial surface. Bacterial cultures were washed once in PBS (0.12 M NaCl, 2.7 mM KCl, 10 mM phosphate, pH 7.4) and resuspended in 100 ul PBS+5% FBS containing 1:1000 dilution of anti-FimC/FimH antiserum (MedImmune Inc.). Binding of primary antibody was allowed to proceed for one hour on ice and followed by three washes with PBS. Bacterial pellets were resuspended in 100 ul of Oregon Green-conjugated goat-mouse IgG (H+L) secondary antibody diluted 1000-fold in PBS+5% FBS and incubated on ice for another hour. After incubation with secondary antibody, bacteria were washed extensively and fixed with 2% glutaraldehyde (in PBS) with 1 μg/ml Hochst stain (Sigma) for 5 min at room temperature (RT). Bacteria were washed once again and resuspended in 100 ul PBS. Five microliters of stained bacteria were spotted on glass microscope slides and allowed to air-dry at room temperature. The staining of FimH on bacterial surfaces was visualized with an Olympus BX60 microscope system.

WT FimCH as well as all of the mutant FimCH proteins were properly localized to the pilus (although data is not shown, it is summarized in Table 8).

6.1.3 Mannose Binding Properties of Mutant FimCH Proteins

FimH allelic variants can be broadly divided into two functional groups, those that bind tri-mannose only and those that also are capable of binding mono-mannose. Mono-mannose binding activity has been correlated to an increased virulence phenotype amongst uropathogenic E. coli. Structural insight into these binding activities was gained by analyzing the effect of each mutation on both mono-mannose and tri-mannose binding. Mannose binding assays were done with purified FimCH complexes as well as FimCH expressed on in tact E. coli.

6.1.3.1 Isolated FimCH Protein

Wild type and mutant FimCH complexes were isolated from E. coli and purified. The protein complexes were tested for mannose binding ability through the use of a number of different assays described below. Data is summarized in Table 8.

Hemagglutination Assay

ORN103/pUT2002 E. coli complemented with FimH expression constructs were induced to express FimH and other gene products in the rest of the type 1 operon. Briefly, bacteria were first grown overnight in shaking incubators at 37° C. On the following day, bacteria were diluted 10-fold and sub-cultured statically again overnight in the presence of 1 μM IPTG. Hemagglutination assays with guinea pig erythrocytes were performed following published protocols (Slonim et al, 1992, EMBO J. 11:4747-56; Hultgren et al., 1990, Mol Microbiol. 4:1311-8 and Duguid et al., 1979, J. Med. Microbiol. 12:213). Inhibition of agglutination by a 10 mM solution of α-methyl mannoside was used to demonstrate that the agglutination was dependent on mannose.

WT FimCH, FimCH S62A, and FimCH N46D gave positive results in this assay. All remaining FimCH mutations abolished the ability to agglutinate erythrocytes (i.e., did not bind mannose on the erythrocyte surface).

Binding to Mannose-Coated Sepharose Beads

Sepharose 6B beads were coated with saturating amounts of D-mannose (Sigma) and resuspended in 0.02% Na azide, 15 mM CaCl₂, 1.25 M NaCl, 10 mM Tri-HCl, pH 7.8. Mono-mannose coated beads were washed extensively and resuspended as 50% (v/v) slurry in 20 mM MES, pH 6.8. Twenty micrograms of FimCH complexes and 100 ul of the mono-mannose beads were used in the binding experiments. Proteins and beads were incubated together for 2 hours in a reaction volume of 200 ul. Unbound proteins were removed and beads were washed three times with PBS. The washed beads were divided into 2 equal portions: to one half, 50 ul of SDS-PAGE loading buffer was added for the determination of bound FimCH and 50 ul of 1% methyl-α-D-mannopyraniosides were added to the other half in attempt to elute bound FimCH. Elution of bound FimCH complexes were allowed to proceed for 40-60 minutes. Following elution, the supernatants were transferred to fresh tubes and proteins in the bound or eluted fractions were resolved on 15% SDS-PAGE gels. SDS-PAGE was performed following standard laboratory protocols. Gels were stained with Coomassie stain according to standard laboratory procedure to visualize the presence of FimCH.

After Coomassie staining and re-hydration, gels were dried onto cellophane sheets. FimCH bands on gels were scanned as digitized images. The quantitation of FimH-band intensity was performed with NIH Image v. 1.62. The relative amounts of FimH proteins on gels were calculated as the integrated intensity of the area surrounding the FimH band. Same area size was used to calculate the intensity of each FimH band.

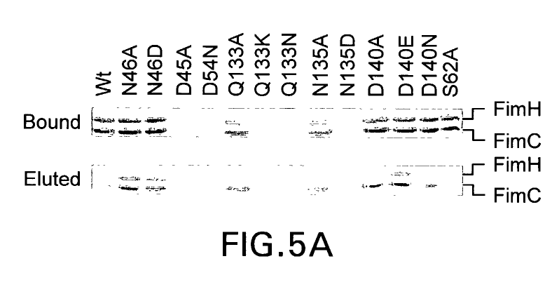

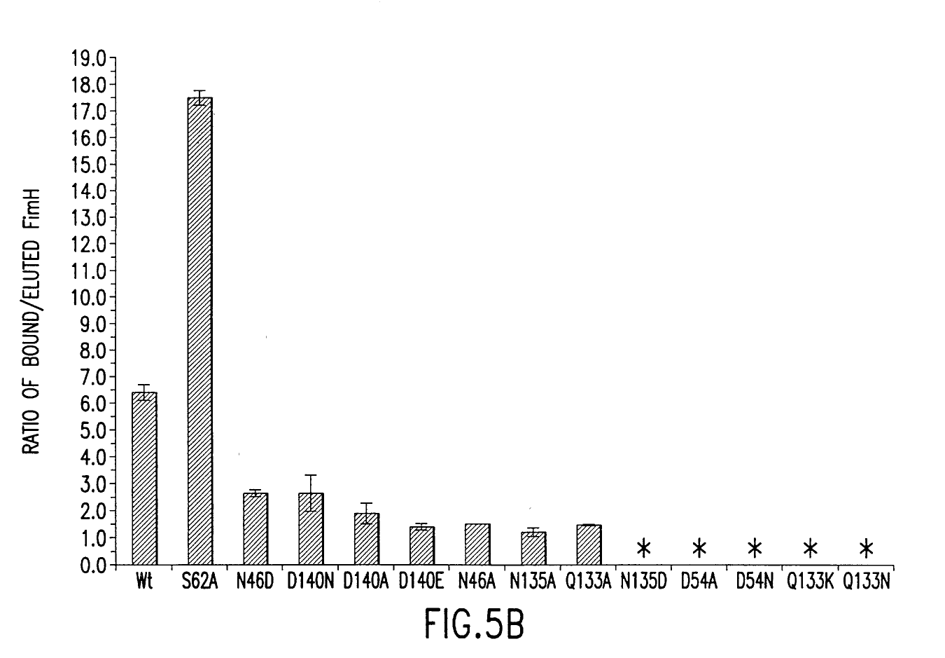

WT FimCH, FimCH S62A, FimCH D140A, FimCH D140N, FimCH D140E, FimCH N46A, and FimCH N46D all bound mono-mannose coated beach; to approximately the same extent. However, the relative amount of FimCH N46D, FimCH D140A, FimCH D140N, FimCH D140E, and FimCH N46A eluted by D-α-mannopyranoside was two- to five-fold greater than the amount of wild type WT FimCH or FimCH S62A eluted from the same amount of beads suggesting that these mutations in FimH decreased its affinity for mono-mannose (FIGS. 5 A-B).

Mannose Affinity Chromatography

In order to evaluate the binding affinities of FimH mutants, an HPLC-format assay was developed using a commercially available methacrylate resin (PE Biosystems) to which a tri-mannose-BSA conjugate (1-3, 1-6-D mannotriose-BSA) or a mono-mannose-BSA conjugate was coupled via epoxide chemistry. The column, which has a bed volume of 0.2 ml, is equilibrated with Phosphate Buffered Saline (PBS, 33.3 mM phosphate, 150 mM NaCl, pH 7.2) and run at a flow rate of 1 ml/minute. Purified FimCH complexes, containing either wild type or mutant FimH, were used in this assay. Samples were diluted to a concentration between 1 and 10 μg/ml using PBS containing 0.5% Tween-20 (PBST). The diluted samples were filtered through a microcentrifuge filter (0.45 μM) at 13000 rpm (10,000 × g) for 3 minutes at room temperature. An injected sample of proteins flowed through the column to allow interactions with the tri- or mono-mannose moieties. An injection of 50 ul of sample is followed by a 0.5-minute PBS wash. The bound FimCH is subsequently eluted with 0.1 M H₃PO₄+0.15 M NaCl for 2 minutes and detected by intrinsic tryptophan fluorescence, using an excitation wavelength of 280 nm and an emission wavelength of 325 nm. Finally, the column is re-equilibrated with PBS for 2.5 minutes. Affinity measurements relative to the wild type FimH can be determined for the bound FimCH complexes based upon the retention time profile.

FimCH Q133A, FimCH N135A, FimCH D140A, FimCH D140N, FimCH D140E, and FimCH N46A were retained on tri-mannose column similarly to WT FimCH. However, none of the mutant FimCH protein complexes could bind to mono-mannose coated beads during this assay.

Solid Phase (ELISA) Binding Assay

One characteristic of the FimCH molecule is its ability to bind to mannose and mannose-derivatives through the FimH portion of the molecule. The mannose solid phase binding ELISA assay was developed to measure this binding, and to assess the binding avidity differences of various mutants of FimCH for mannose derivatives. This assay exploits the mannose binding function of the FimH region of the molecule.

Immulon 4 plates were coated overnight at 4° C. with 0.1 μg/well of mono-mannose- or tri-mannose-BSA. On the following day, wells were blocked with 300 ul/well of PBS+1% BSA+0.02% Sodium azide for 1 hour at 37° C. followed by three washes with PBS+0.05% Tween-20 (PBST). FimCH samples were diluted in PBS+0.05% Tween-20+0.1% BSA. One hundred microliters of diluted protein samples were added into each well. Plates were incubated at 37° C. for 1 hour. After incubation with FimCH complexes, wells were washed three times with PBST. Subsequently, biotin-conjugated anti-FimC monoclonal antibody was added to each well and plates were incubated again at 37° C. for 1 hour. At the end of incubation, wells were washed as above and horseradish peroxidase-conjugated streptavidin (1:1000 dilution) (Tropix) was added. Following a 30 minute incubation at 37° C., wells were washed again as above. ELISA reaction was developed with TMB substrate at room temperature for 10 minutes and stop reaction with 50 ul/well of 2N H₂SO₄. Reaction plates were read on SOFTmax at 450 nm.

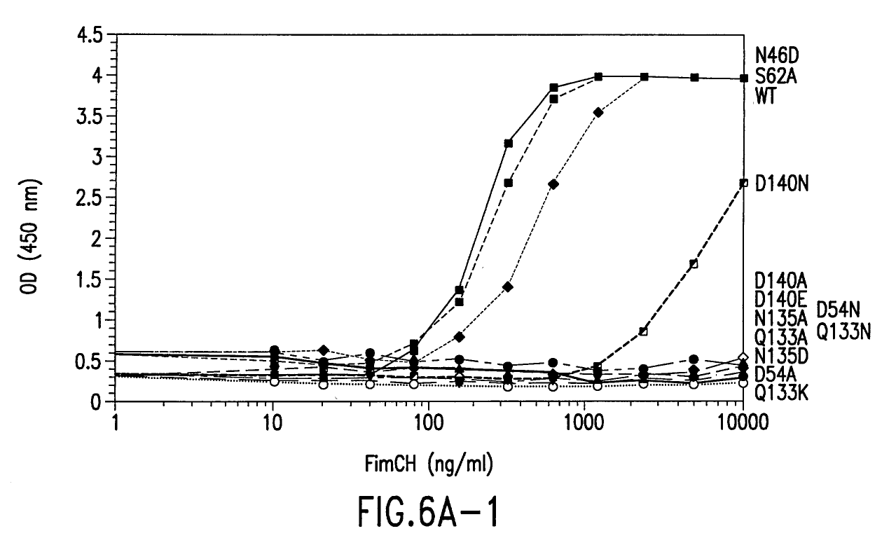

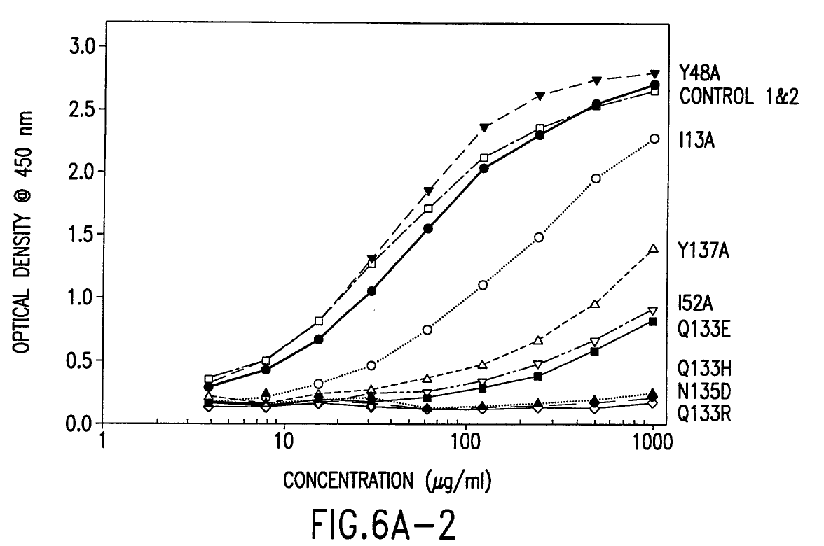

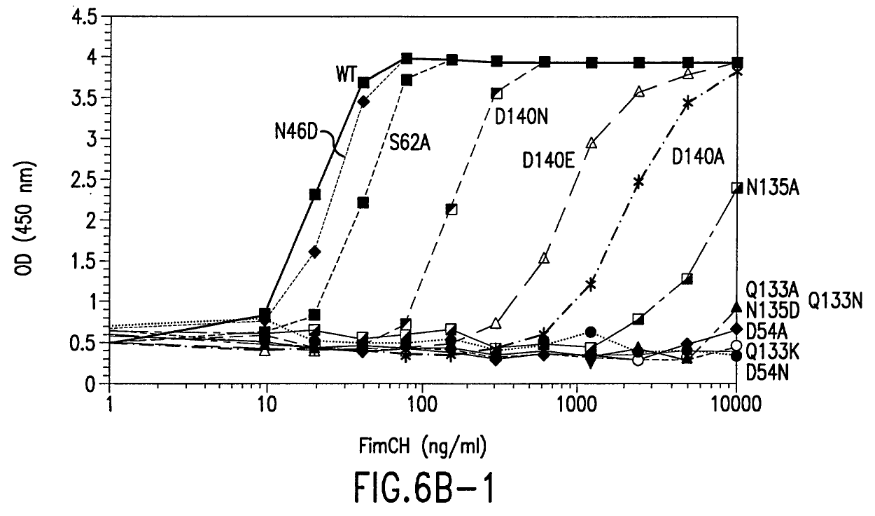

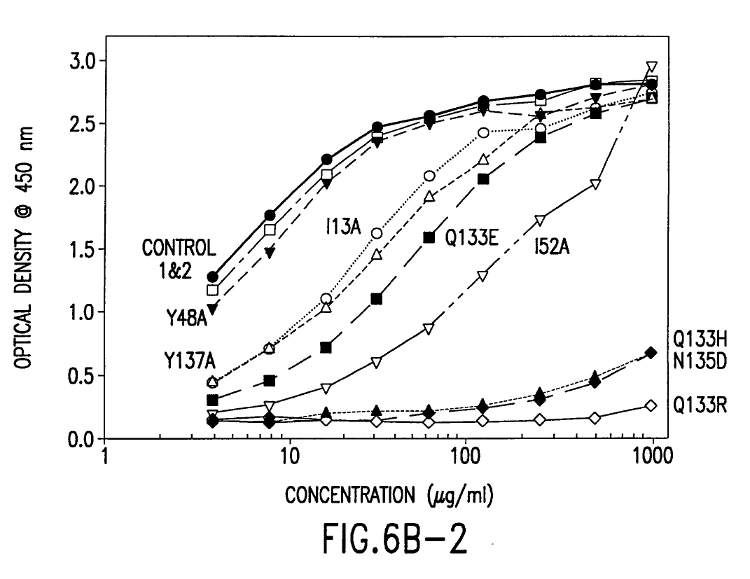

Wild type FimCH was able to bind tri-mannose approximately 10 times better than mono-mannose as measured by ELISA. A two fold reduction in the relative binding of FimCH N46D to mono-mannose was also detected by ELISA however binding to tri-mannose seemed to be unaffected by the mutation. Binding to mono-mannose in the ELISA by FimCH Q133A, FimCH N135A, FimCH D140A, FimCH D140N, FimCH D140E, and FimCH N46A was undetectable with the exception of FimCH D140N, which showed very low levels of binding. Interestingly, although mutations in residue 140 greatly reduced (FimCH D140N) or abolished (FimCH D140A and FimCH D140E) mono-mannose binding in the ELISA assay, they retained their ability to bind tri-mannose, albeit at reduced levels compared to the wild type protein. (FIGS. 6 A-B)

6.1.3.2 FimCH Protein Expressed on E. coli

E. coli strain PmmB66 was transfected with cDNA encoding the various FimH mutants. Because PmmB66 does not endogenously express FimH, all of the FimCH complexes on its surface will contain the FimH mutant protein. Mannose binding ability of the mutant FimCH protein when in the context of a cell surface receptor was examined by the following whole cell solid phase mannose binding assay.

Each well of an Immulon-4 plate (Dynex Technologies, Chantilly, Va.) was coated with 2.5 μg/ml of mono-mannose or tri-mannose-BSA (V-labs, Covington, La.) in Carbonate Coating Buffer overnight at 4° C. The wells were aspirated and then blocked with PBS+1% BSA (300 ml/well) by incubation at 37° C. for 1 hour. Plates were then washed three times with PBS+0.1% Tween+0.1% BSA. The E. coli expressing either wild type or mutant FimCH (8.0×10⁷ CFU/ml) were added to each well and incubated at 37° C. for 1 hour, and then washed extensively. Bound bacteria were detected with a 1:400 dilution of a polyclonal anti-E. coli (all antigens)-peroxidase conjugated antibody (BioDesign, Inc., Kennebunk, Me.; catalog no. B65004R). After washing three times with PBS+0.1% Tween+0.1% BSA, the TMB substrate (100 ml/well) was added and incubated at ambient temperature for optimal time before stopping with 2N H₂SO₄. OD₄₅₀ readings were taken to quantify the amount of bacteria bound to the mannose.

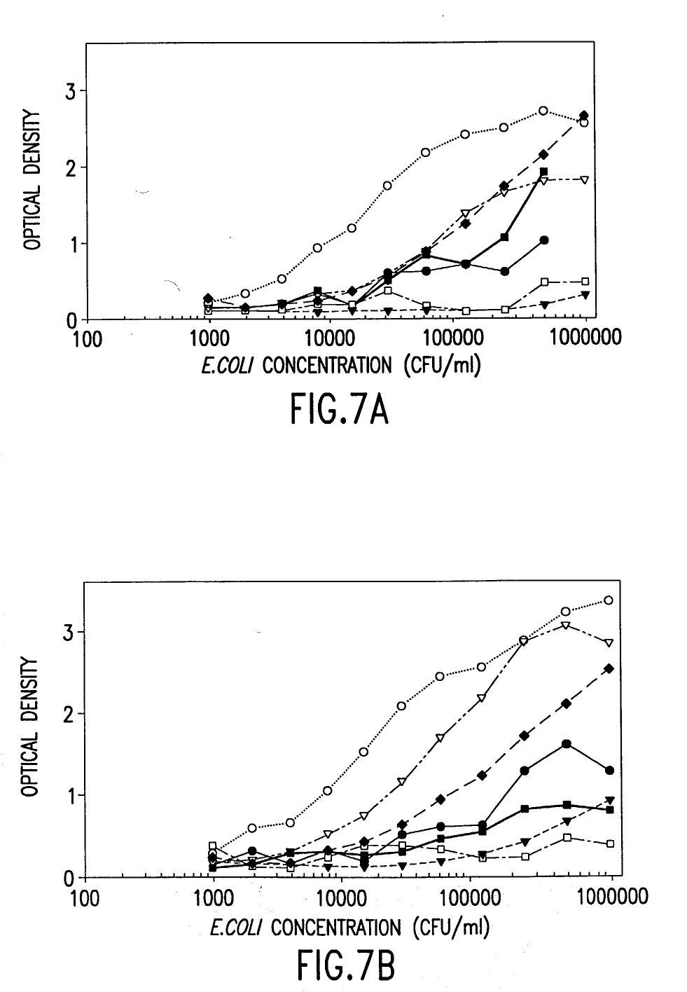

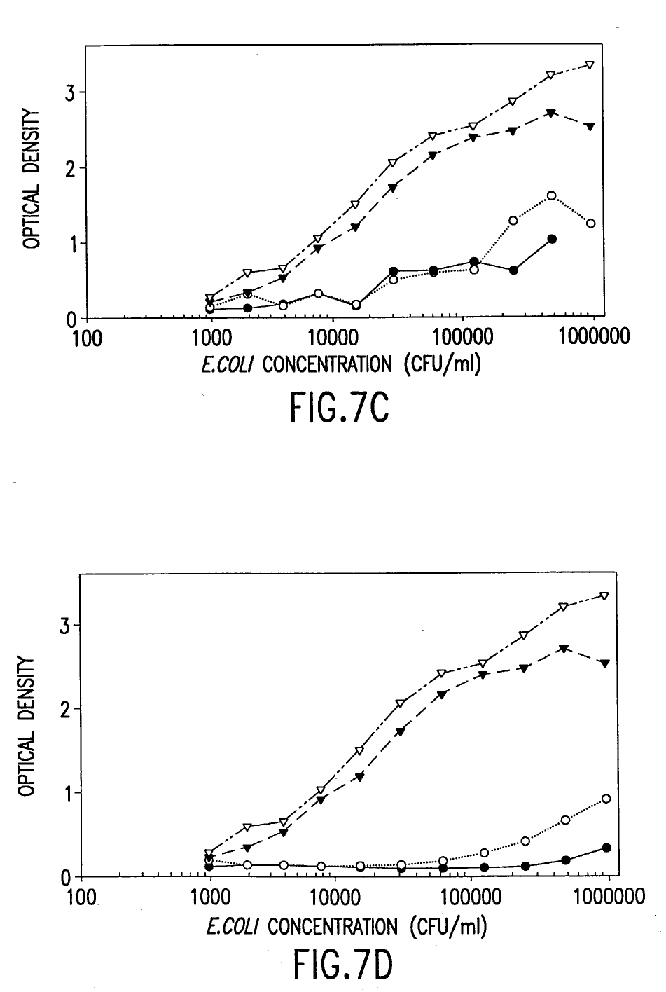

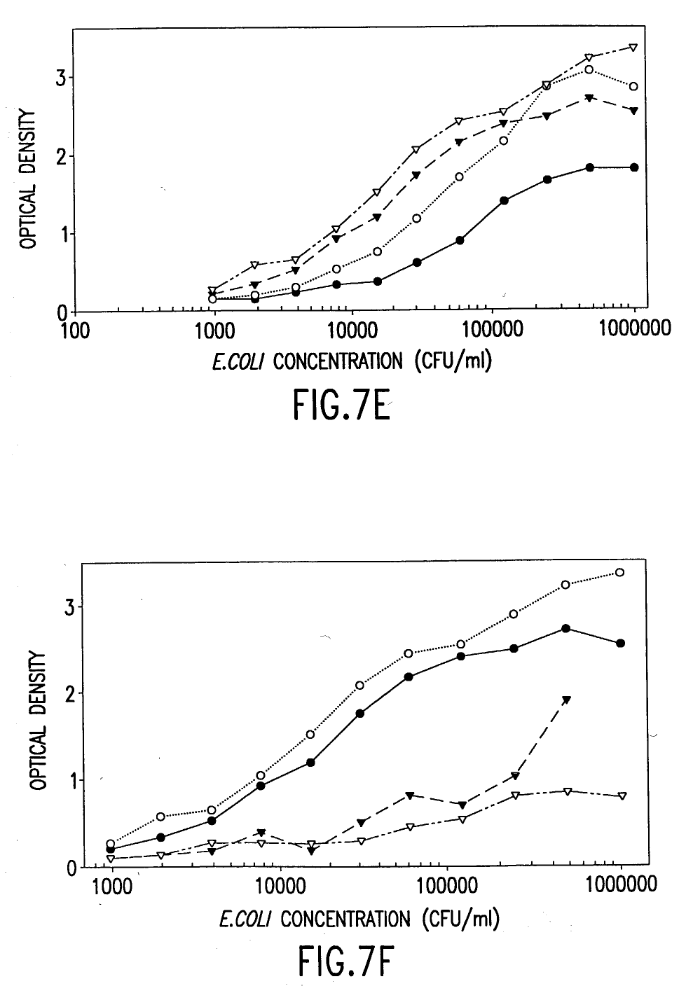

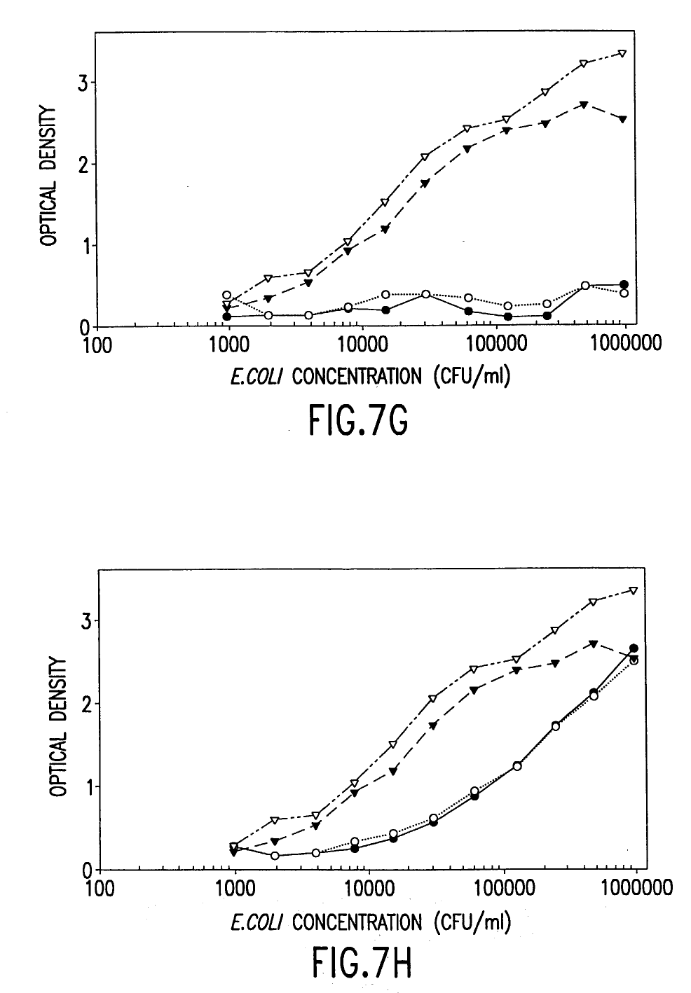

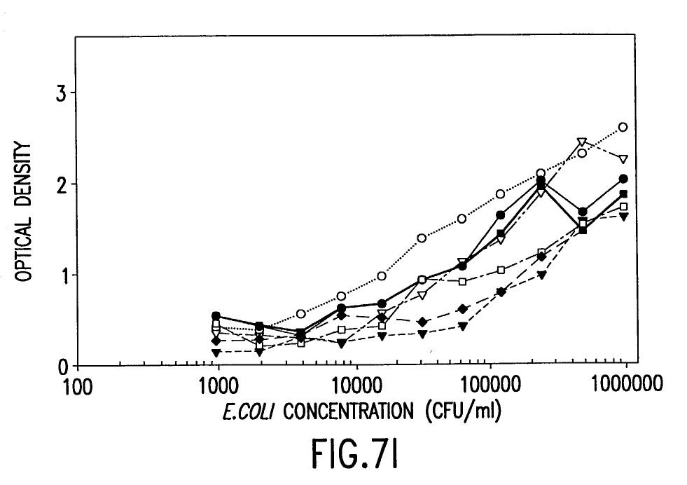

FimCH N46D could bind tri-mannose at near wild type levels but had a decrease in its ability to bind mono-mannose (FIG. 7E). FimCH S62A could bind mono- and tri-mannose equally well, but at a level that was somewhat decreased from wild type ability (FIG. 7H). No significant binding could be detected for FimCH N46A, FimCH D140E, and FimCH Q133K (FIGS. 7D, 7F, and 7G) on either mono- (or tri-mannose. These results are similar to those obtained when testing mannose-binding ability of isolates mutant FimCH proteins (see Section 6.1.3.1).

As a control, plates were coated with the polyclonal anti-E. coli antibody and then exposed to E. coli expressing the different FimCH mutant proteins.. FIG. 7I shows that the polyclonal antibody can bind to each of the mutant-expressing E. coli equally well. This indicates that any differences in the amount of E. coli detected in FIGS. 7A-H reflect a true difference in mannose binding instead a of a technical difficulty with the detection method.

6.1.4 Adherence and Invasion Assays

AAEC185/pUT2002 transformed with FimH expression plasmids were used to assay FimH-mediated bacterial adherence and invasion into the human bladder cell line 5637 (ATCC # HTB-9). Bacteria were cultured as described above for type 1 pili expression. Adherence and invasion assays were performed following published protocols with a minor modification (Elsinghorst & Weitz, 1994, Infect Immun. 62:3463-71; Martinez et al., 2000, EMBO J. 2000 19:2803-12). Instead of a two-hour infection step, bacteria were incubated for one hour to allow for binding and entry into bladder cells.

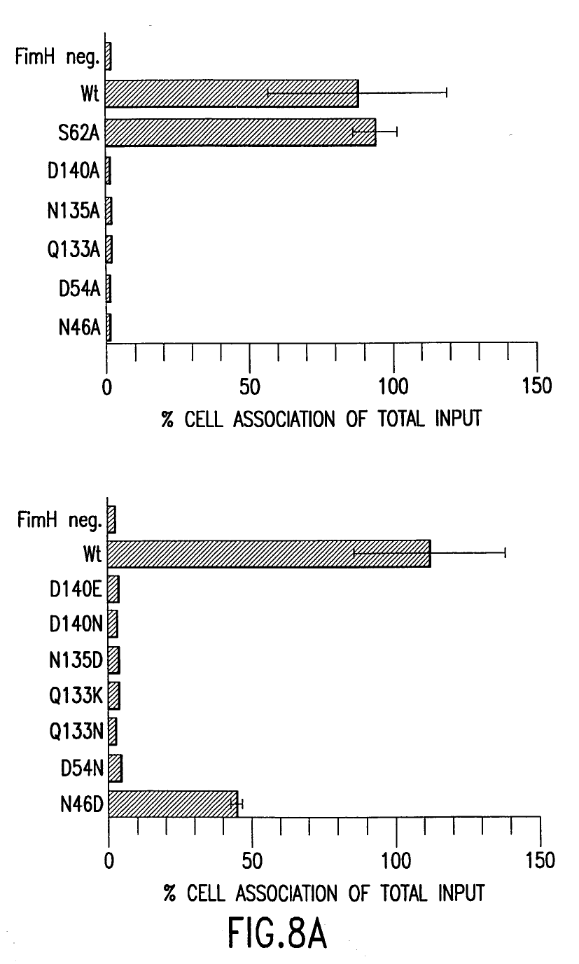

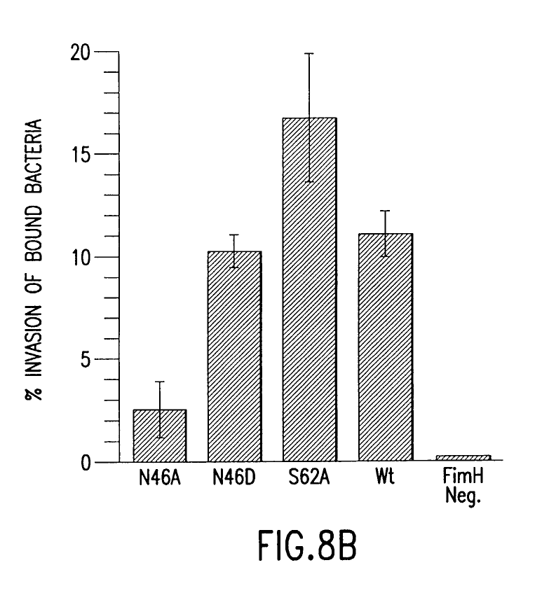

WT FimCH, FimCH S62A, and FimCH N46D could adhere and invade the bladder cells (although FimCH N46D had a 2-fold decrease in ability when compared to WT FimCH). All of the remaining mutant FimCH proteins, however, had no ability to adhere or to bind bladder cells (FIG. 8A). However, all of those E. coli expressing a FimCH complex competent to adhere to 5637 cells, could also invade (FIG. 8B).

E. coli expressing FimCH proteins were also tested for the ability to bind human bladder tissue sections. AAEC185/pUT2002 transformed with FimH expression plasmids were used to assay FimH-mediated bacterial adherence to tissue sections. Bacteria were cultured as described above for the optimal expression of type 1 pili. In situ binding to human bladder tissues was performed similarly to previously described protocol with minor modifications (Striker, 1995, Adv Exp Med Biol. 385:141-2; Falk et al. 1993, Proc. Natl. Acad. Sci. USA, 90:2035-2039). Briefly, overnight cultures were diluted to the same concentration (OD₆₀₀ 1) and 1 ml of each diluted bacteria was labeled with fluorescein isothiocyanate (FITC) as described (Falk et al., 1993, Proc. Natl. Acad. Sci. USA, 90:2035-2039). Labeled bacteria were resuspended in 1 ml blocking buffer (PBS+0.25% BSA+0.05% Tween-20). Non-diseased human bladder sections were obtained from the surgical pathology and autopsy files of the Department of Pathology at Washington University and deparaffinized following published protocol Falk et al., 1993, Proc. Natl. Acad. Sci. USA, 90:2035-2039. Human bladder tissues on microscope slides were incubated with 100 ul of freshly FITC-labeled bacteria for 2 hours at room temperature in a humidified chamber. Following bacterial binding, slides were washed extensively with PBS, and fixed for 5 minutes with 2.5% glutaraldehyde in PBS. After fixation, slides were counterstained with 1 μg/ml Hoechst stain for 5 minutes. Upon mounting with cover slips, slides were dried overnight at room temperature in the dark. Visualization of bound bacteria was performed on an Olympus BX60 microscope system.

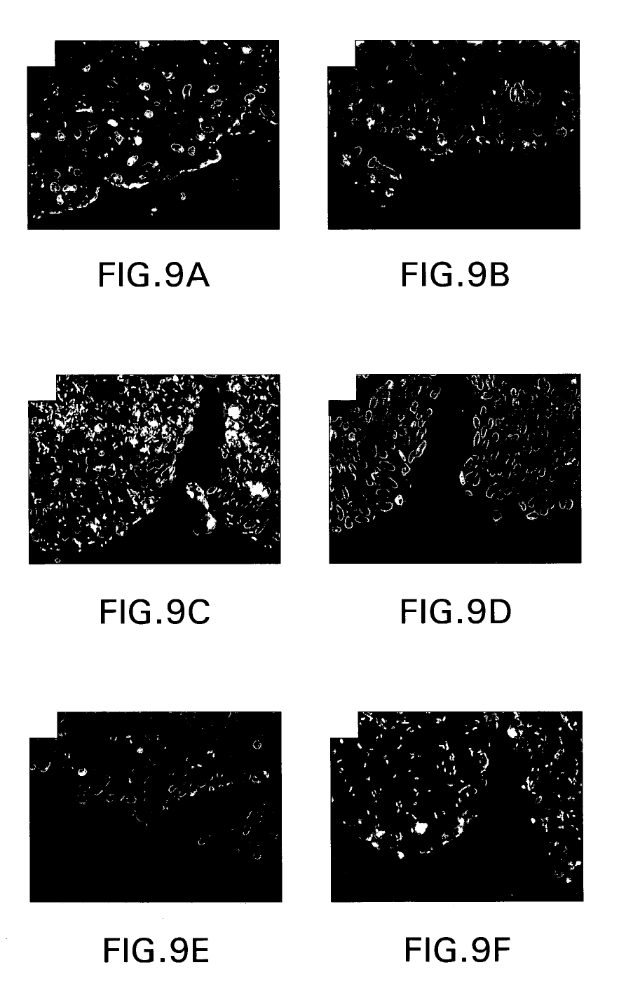

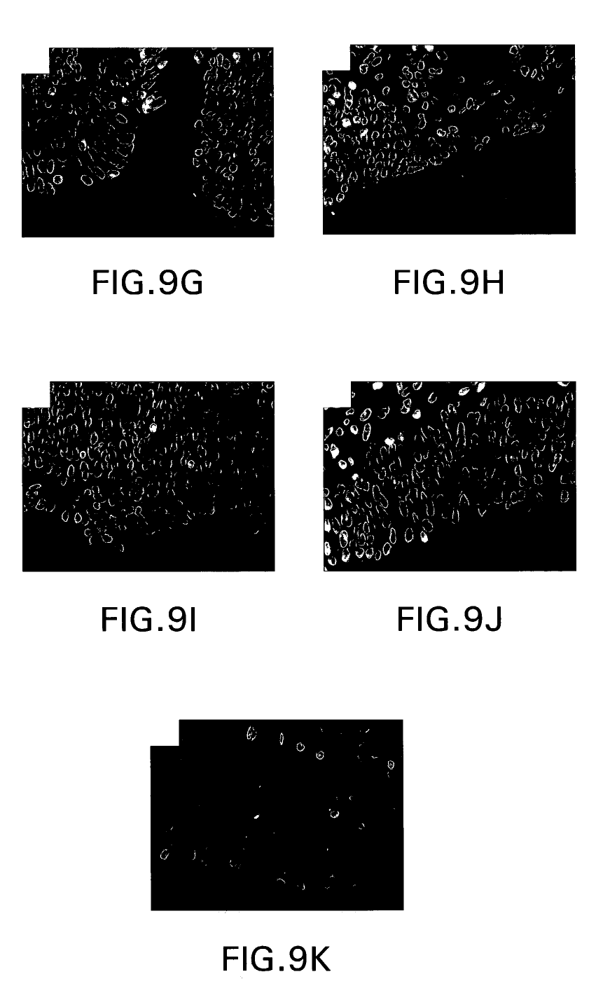

Both WT FimCH and FimCH S62A mediated a high level of tissue binding in a mannose-inhibitable fashion (FIGS. 9A-D). Bacteria were seen binding to the luminal surfaces of the bladder sections as well as the sub-layers of the bladder epithelium. FimCH N46D could adhere and invade the bladder cells albeit it had a 2-fold decrease in ability when compared to WT FimCH (FIGS. 9E-F). Binding mediated by FimCH N46D was inhibited by soluble mannose (FIG. 9G). None of the other mutants tested showed significant binding or invasion. (FIGS. 9H-K). (Data is summarized in Table 8).

TABLE 8

| | | | Mannose Binding | | | | | | | | | |
|---|---|---|---|---|---|---|---|---|---|---|---|---|
| | | | | | | | | | ELISA | | | |
| | | | Affinity | | | | ELISA | | (with FimCH | | | |
| | Pilus | | Chromatography | | Beads | | (with purified FimCH) | | on E. coli) | | Bladder | |
| FimH | Local- | | Tri- | Mono- | Tri- | Mono- | Tri- | Mono- | Tri- | Mono- | Cell | |
| protein | ization | HA | mannose | mannose | mannose | mannose | mannose | mannose | mannose | mannose | Adherence | Invasion |
| WT | + | + | + | + | + | + | +(3) | + | + | + | + | + |
| I13A | + | nd | nd | nd | nd | | + | + | nd | nd | nd | nd |
| S62A | + | + | nd | nd | nd | + | nd | nd | +(7) | +(7) | + | + |
| N46D | + | + | nd | nd | nd | +(1) | + | +(5) | + | +/− | +(5) | + |
| N46A | + | − | nd | nd | nd | +(1) | nd | − | − | − | − | − |
| Y48A | + | nd | nd | nd | nd | nd | + | + | nd | nd | nd | nd |
| I52A | + | nd | nd | nd | nd | nd | + | +/−(6) | nd | nd | nd | nd |
| D54A | + | − | − | − | − | − | − | − | nd | nd | − | − |
| D54N | + | − | − | − | − | − | − | − | nd | nd | − | − |
| Q133K | + | − | − | − | − | − | − | − | − | − | − | − |
| Q133A | + | − | + | nd | + | +/−(2) | nd | − | nd | nd | − | − |
| Q133N | + | − | − | − | − | − | − | − | nd | nd | − | − |
| Q133E | + | nd | nd | nd | nd | nd | + | +/−(6) | nd | nd | nd | nd |
| Q133H | + | nd | nd | nd | nd | nd | − | − | nd | nd | nd | nd |
| Q133R | + | nd | nd | nd | nd | nd | − | − | nd | nd | nd | nd |
| N135A | + | − | + | nd | + | +/−(2) | nd | − | nd | nd | − | − |
| N135D | + | − | − | − | − | − | − | − | nd | nd | − | − |
| Y137A | + | nd | nd | nd | nd | nd | + | +/−(6) | nd | nd | nd | nd |
| D140E | + | − | + | nd | nd | +(1) | +(4) | − | − | − | − | − |
| D140A | + | − | + | nd | nd | +(1) | +(4) | − | nd | nd | − | − |
| D140N | + | − | + | nd | nd | +(1) | +(4) | +/− | nd | nd | − | − |

nd indicates not determined
(1)WT and mutant protein bind to mono-mannose beads in equal amounts; mutant protein elutes with a-D-mannopyranoside 2-5 fold more easily
(2)mutant protein binds mono-mannose beads less well than WT protein and elutes with α-D-mannopyranoside 4-5 fold more easily
(3)WT protein binds tri-mannose 10 fold better than mono-mannose as assayed by ELISA
(4)mutant proteins bind tri-mannose at reduced levels when compared to WT protein (D140N binds tri-mannose as well as WT binds mono-mannose)
(5)WT protein binds 2 fold better than mutant protein
(6)mutant protein binds higher concentration of mono-mannose at 30%-50% WT levels
(7)mutant protein binds mono- and tri-mannose equally well but decreased from WT levels

6.1.5 Naturally Occurring FimH Mutant

All of the mutations in the mono-mannose binding pocket completely abolished binding to bladder epithelium except for the N46D mutation. The N46D mutation reduced binding to bladder cells by about 50%. It retained the ability to bind tri-mannose with the same relative affinity as wild type FimH but had approximately a 50% reduced affinity for mono-mannose. Thus, mono-mannose but not tri-mannose binding appears to be strictly correlated with the physiologically relevant function of FimH in binding bladder epithelium. Since the amide oxygen that binds O6 is left intact in the N46D mutant, the 50% reduction in mono-mannose and bladder binding is presumably a result of the inability to stabilize the pocket to the same degree as the wild type. Thus, even the slightest change in the mannose binding pocket, in an atom that does not directly bind mannose, still significantly reduces binding, emphasizing why the pocket is invariant mannose 200 uropathogenic isolates (see, e.g., FIG. 3).

Enterohemorragic E. coli (EHEC) are the cause of hemolytic uremic syndrome which results in acute kidney failure (Noel et al., 1997, Dig. Dis. 15:67-91). This syndrome is thought to be the effect of the Shiga toxin, that enters the blood stream and locates to the kidney due to its receptor binding specificity (Kiyokawa et al., 1998, J. Infect. Dis. 178:178-184; Cooling et al., 1998, Infect. Immun. 66:4355-4366). Although EHEC possess the type 1 pilus gene cluster, there is a lack of an association of EHEC strains with urinary tract infections. Interestingly, an inspection of the FimH gene sequences from three different enterohemorragic strains revealed that the binding pocket residue Asn135 was changed to a lysine (this sequence is depicted in FIG. 3 as EC189). A lysine at this position would be predicted to exclude mannose from the binding pocket. A dysfunctional mono-mannose binding pocket would render EHEC unable to colonize the bladder and establish an infection. This may represent a natural selection for a less virulent phenotype since colonization of the urinary tract would lead to a direct delivery of the toxin to the kidney causing drastic and rapid consequences to the host.

### 6.2 Example 2

### Production of Antibodies

6.2.1 Polyclonal Antibodies

The immunogenicity of purified FimCH variant proteins were assessed by measuring immunoglobulin G (IgG) titer to FimH T3. FimH T3 is a hisitidine-tagged fusion protein composed of the first 165 amino acids of the mature (279 amino acids) FimH protein.

C3H/HeJ mice were immunized on day 0 (primary immunization) and booster immunized during week 4 with one of the 7 purified antigens: wild type FimCH (from strain J96), wild type FimCH (vaccine composition), FimCH D140E, FimCH N46D, FimCH Q133K, FimCH Q133E, and FimCH Q133H. Injections were at doses of 4.0, 1.6, 0.64, and 0.26 μg in MF59 adjuvant (Chiron, Emeryville, Calif.).

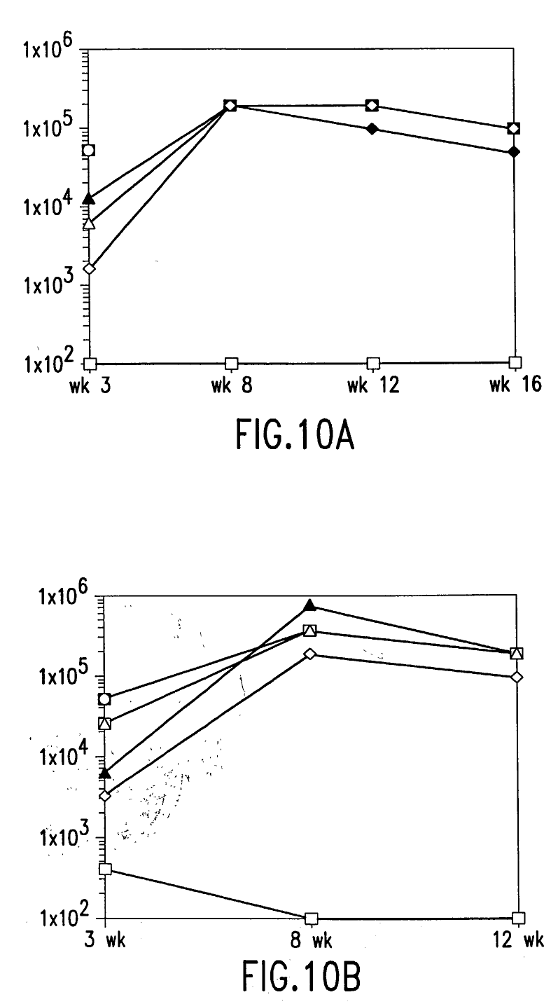

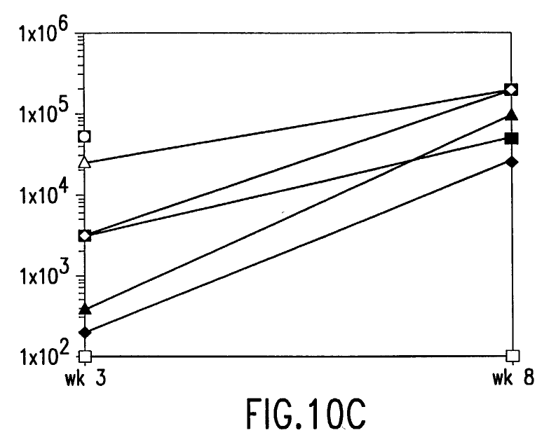

Samples from individual mice treated identically were pooled for serological analysis and diluted 1:100 before serial dilution. Antibody responses were assessed by an ELISA with purified FimH T3 as the capture antigens. The purity of the protein preparations of the capture antigen was 95% pure for FimH T3. In all cases the protein preparations were free of any lipopolysaccharide contaminants. Data for immune responses of such mice to the various FimH adhesins is in FIGS. 10A-C.

Mice vaccinated with FimCH N46D and FimCH D140E showed comparable response to FimCH T3 by ELISA both at 3 weeks (pre-boost) and at 8 weeks (4 weeks post boost) at all doses when compared to wild type FimCH (FIG. 10A and 10B).

Interestingly, mice vaccinated with FimCH Q133K protein induced titers to FimH T3 at 3 weeks (pre-boost) that were approximately 20 fold lower than titers to wild type FimH at all doses. However, titers from the FimCH Q133K immunized mice did increase following the boost at 4 weeks and were now comparable to the wild type protein (FIG. 10C). This was true at all doses.

6.2.2 Monoclonal Antibodies

Monoclonal antibodies (MAB) were made directed against purified WT FimCH or FimCH Q133K protein using standard techniques well known in the art. Various proteins were used at a 1 μg/ml concentration as capture antigens in an ELISA assay to determine the epitope of each monoclonal antibody clone. Capture antigens included FimC alone (Table, row 1), wild type and mutant FimCH complexes (Table 9 rows 2-8), and truncated FimH proteins (rows 9-11; T3 is a histidine tagged N-terminal lectin binding domain of FimH from amino acid residues 1-184; T2B is the N-terminal lectin binding domain of FimH from amino acid residues 1-178). FimH specific clones were identified based on positive reactivity with the FimCH or FimCH Q133K complex and a negative reactivity with FimC alone by ELISA (Table 9, compare rows 1-3). Clones 1A7, 1C10, 3E11, and 1F2 bind an epitope on FimH while clones 2B2 and 4G3 bind an epitope on FimC. Interestingly, not all MAB clones that bind to FimH do so equally well. For example, clone 1A7 bound FimCH Q133K better than WT FimCH and did not bind FimCH N135D and FimCH D54A at all (Table 9, rows 2-5) while clone 1C10 bound all FimH-containing complexes equally well (Table 9, rows 2-8).

TABLE 9
Binding specificity of monoclonal antibodies

| | | | | | | | positive |
|---|---|---|---|---|---|---|---|
| | 1A7 | 1C10 | 3E11 | 1F2 | 4G3 | 2B2 | control |
| 1 - FimC | 0.038 | 0.037 | 0.039 | 0.04 | 0.553 | 0.697 | 0.982 |
| 2 - FimCH | 0.328 | 0.624 | 0.098 | 0.845 | 0.793 | 1.04 | 1.1 |
| WT | | | | | | | |
| 3 - FimCH | 0.504 | 0.710 | 0.318 | 0.555 | 0.616 | 0.900 | 1.1 |
| Q133K | | | | | | | |
| 4 - FimCH | 0.04 | 0.668 | 0.038 | 0.643 | 0.694 | 0.951 | 1.1 |
| N135D | | | | | | | |
| 5 - FimCH | 0.055 | 0.600 | 0.042 | 0.735 | 0.752 | 1.02 | 1.17 |
| D54A | | | | | | | |
| 6 - FimCH | 0.476 | 0.734 | 0.370 | 0.761 | 0.734 | 0.988 | 1.1 |
| Q133A | | | | | | | |
| 7 - FimCH | 0.351 | 0.757 | 0.160 | 0.700 | 0.705 | 0.948 | 1.1 |
| Q133N | | | | | | | |
| 8 - FimCH | 0.093 | 0.710 | 0.05 | 0.828 | 0.750 | 1.01 | 1.15 |
| D140A | | | | | | | |
| 9 - FimH | 0.616 | 0.995 | 0.204 | 0.104 | 0.469 | 0.047 | 1.1 |
| T3 | | | | | | | |
| 10 - FimH | 0.283 | 1.0 | 0.180 | 0.187 | 0.621 | 0.046 | 1.1 |
| T2B Q133K | | | | | | | |
| 11 - FimH | 0.334 | 1.04 | 0.092 | 0.092 | 0.116 | 0.045 | 1.2 |
| T2B WT | | | | | | | |

Further information regarding the type of epitope recognized by each MAB clone was obtained by western blot analysis as well as by ELISA under urea-denaturing conditions. Western blotting was carried out according standard laboratory protocols also. Briefly, proteins in SDS-PAGE gels were transferred to PVDF membranes (Schleicher & Schuel) and blocked for one hour in blocking buffer consisting of TBST (500 mM NaCl, 0.05% Tween-20, 20 mM Tri-HCL, pH 7.5)/5% nonfat dry milk/3% bovine serum albumin (BSA). Blots were washed briefly in TBST and incubated with anti-FimC/FimH mouse antiserum diluted 1000-fold in blocking buffer for one hour. Following primary antiserum incubation, blots were washed three times for 5 min each with TBST and incubated for another hour with alkaline phosphatase (AP)-conjugated goat α-mouse IgG (whole molecule) secondary antibody (Sigma) diluted 2000-fold in blocking buffer. Subsequently, blots were washed four times for 5 min each with TBST and once with developer buffer (100 mM NaCl, 5 mM MgCl, 100 mM Tri-HCl, pH 9.5) and then developed with 0.04% NBT+0.02% BCIP (diluted in developer buffer).

The results are summarized in Table 10. Briefly, 1A7 and 1C10 cannot recognize FimCH Q133K protein when the protein is denatured indicating that a conformational epitope is recognized. Alternatively, 1F2 can recognize denatured protein indicating that a linear epitope is recognized.

TABLE 10
Characterization of MAB against FimCH Q133K

| | | | ELISA with urea-denatured |
|---|---|---|---|
| MAB clone | epitope | western blot | protein |
| 1A7 | bind FimH | no | no |
| 1C10 | bind FimH | weak | no |
| 3E11 | bind FimH | nd | nd |
| 2B2 | bind FimC | nd | nd |
| 1C8 | bind FimC | strong | nd |
| 1F2 | bind FimH | strong | yes |

nd indicates not determined

### 6.3 Example 3

### Inhibitory Properties of Polyclonal Anitibodies

6.3.1 In Vitro

Functional inhibitory properties of polyclonal antibodies were measured by the ability to block binding of type 1 piliated bacteria (E. coli strain NU14) to guinea pig erythrocytes in a hemagglutination assay and by the ability to inhibit E. coli binding to block binding of type 1 piliated bacteria (E. coli strain NU14) to transformed human bladder J82 cell line.

Hemagglutination Assay

The bacteria were directly labeled with fluorescein isothiocyanate (FITC) and incubated with the antibody to be assayed for 30 minutes at 37° C. The bacteria/antibody mixture was then added to the erythrocytes and allowed to incubate. After multiple washes, mean channel fluorescence was used as an indicator of the amount of FITC-labeled bacteria remaining (and thereby is an indication of the strength of the interaction between the FimCH complex on the E. coli and mannose). Lysis II software (Becton Dickinson Immunocytometry Systems) was used for analysis of data.

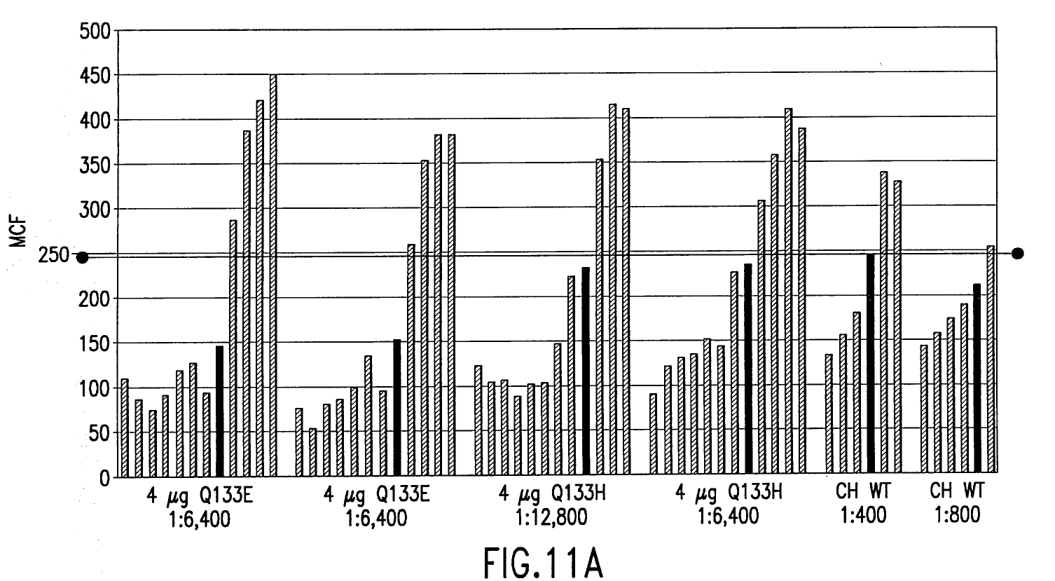

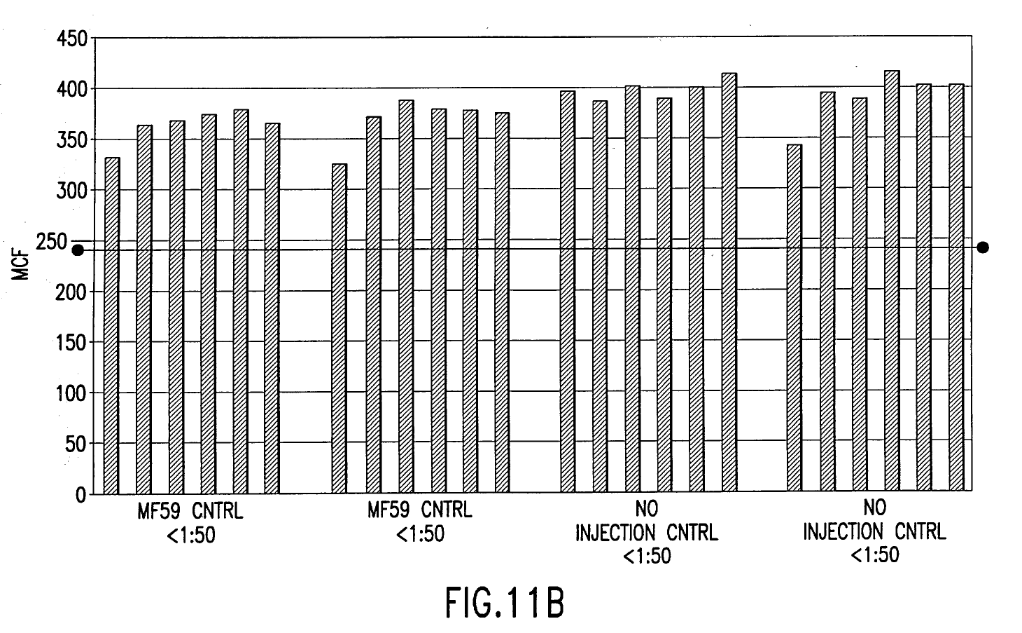

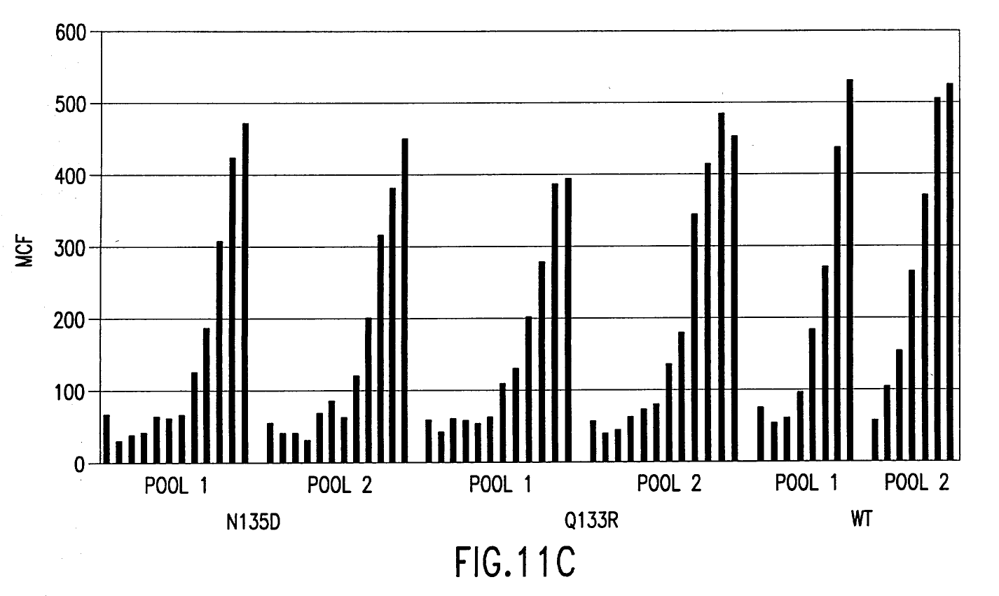

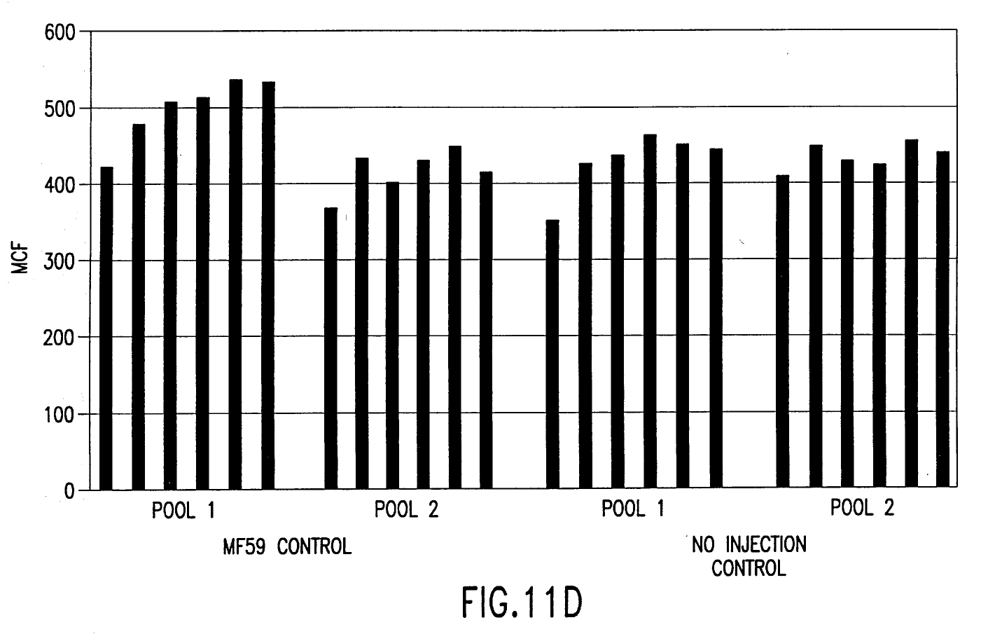

FIG. 11 shows the results from the hemagglutination assay. Increasing dilutions of polyclonal antibodies were used in a set of parallel experiments. Preincubation with polyclonal antibodies raised against FimCH Q133 E, FimCH Q133H, FimCH Q133R, FimCH N135D, and WT FimCH inhibited bacteria binding to the erythrocytes very strongly. Polyclonal antibodies raised against FimCH Q133E and FimCH Q133H were inhibitory at greater dilutions than those used for polyclonal antibodies raised against wild type protein (8-32 times more diluted). Control antiserum from animals that were either not immunized or immunized with MF59 adjuvant alone showed no inhibition.

Inhibition of Binding to Bladder Cells

Functional inhibitory properties of antibodies were measured by the ability to block binding of type 1 piliated bacteria (E. coli strain NU14) to transformed human bladder J82 cell line (American Type Culture Collection Accession Number HTB1). The bacteria were directly labeled with fluorescein isothiocyanate (FITC) and incubated with the antibody to be assayed for 30 minutes at 37° C. The bacteria/antibody mixture was then added to 1×10⁶ bladder cells at a ratio of 250 bacteria/cell. After multiple washes. samples were assayed by flow cytometry (FACStar PLUS; Becton Dickinson, San Jose, Calif.) as described in Langermann et al. (1997, Science 276:607-11; which is hereby incorporated by reference in its entirety). Mean channel fluorescence was used as an indicator of FITC-labeled bacteria bound to the J82 bladder cells. Lysis II software (Becton Dickinson Immunocytometry Systems) was used for analysis of data.

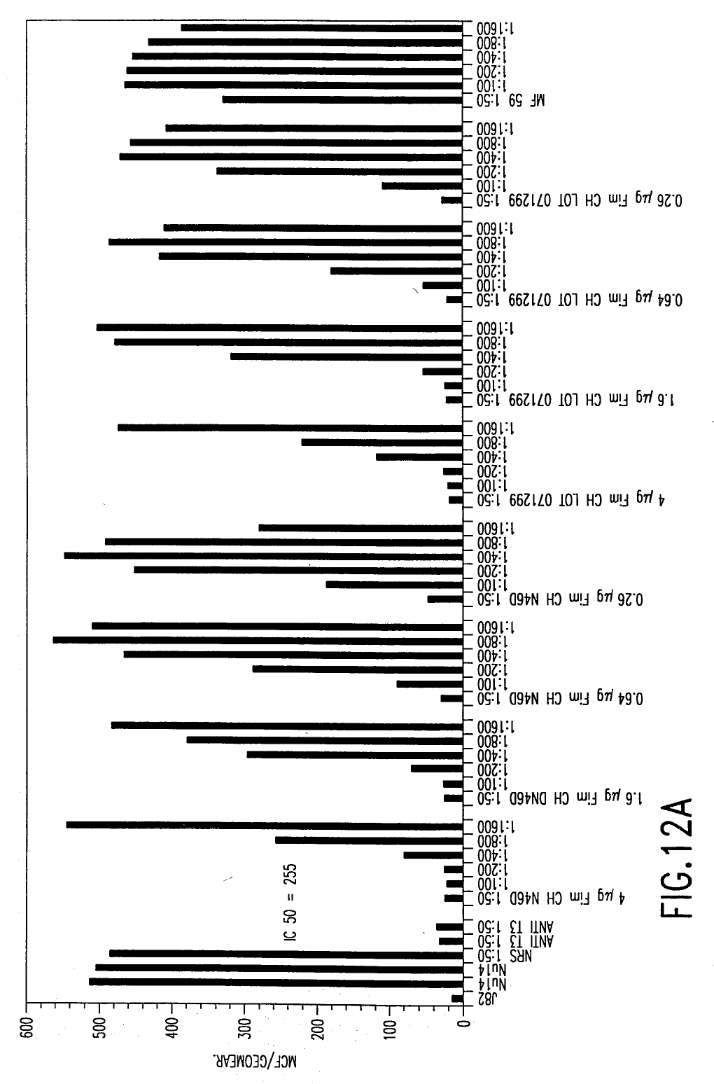

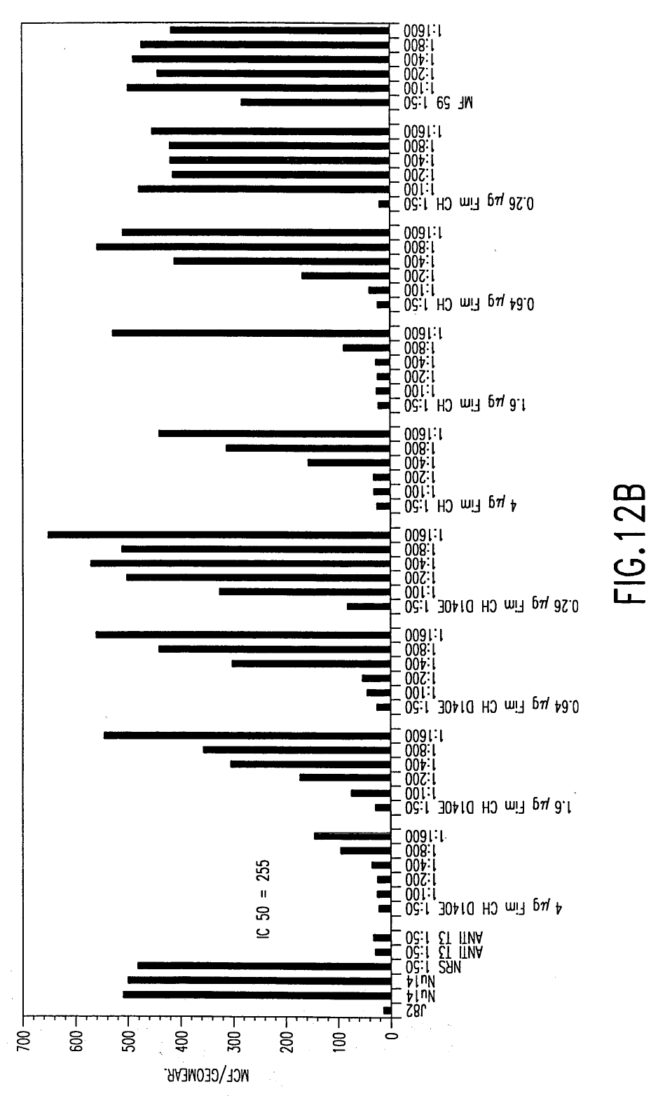

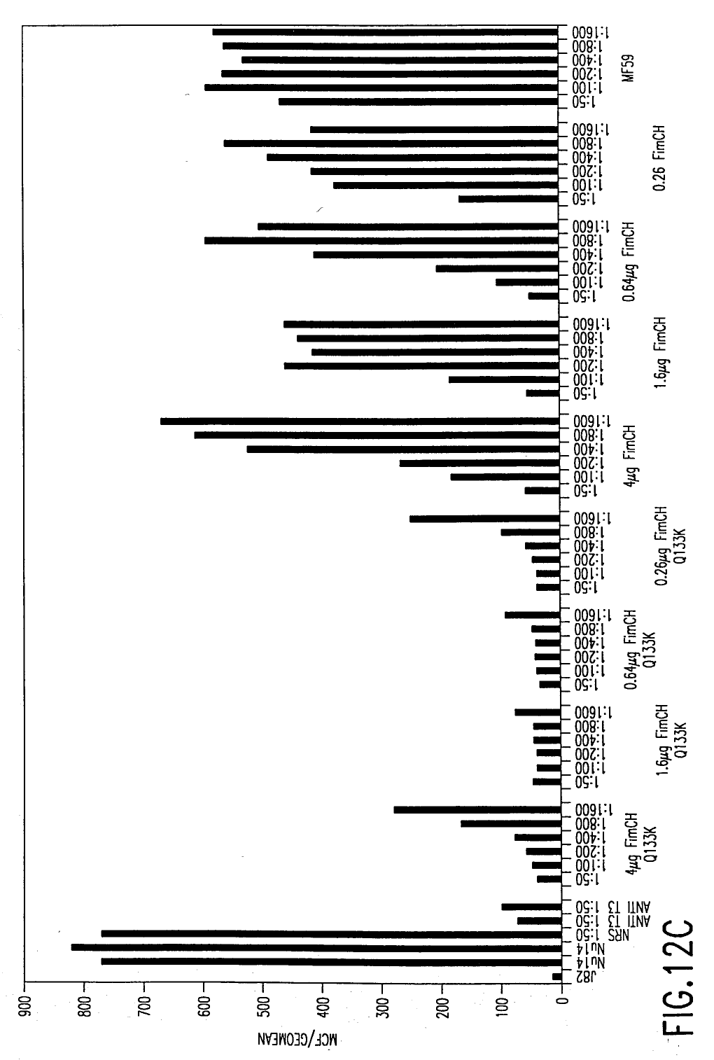

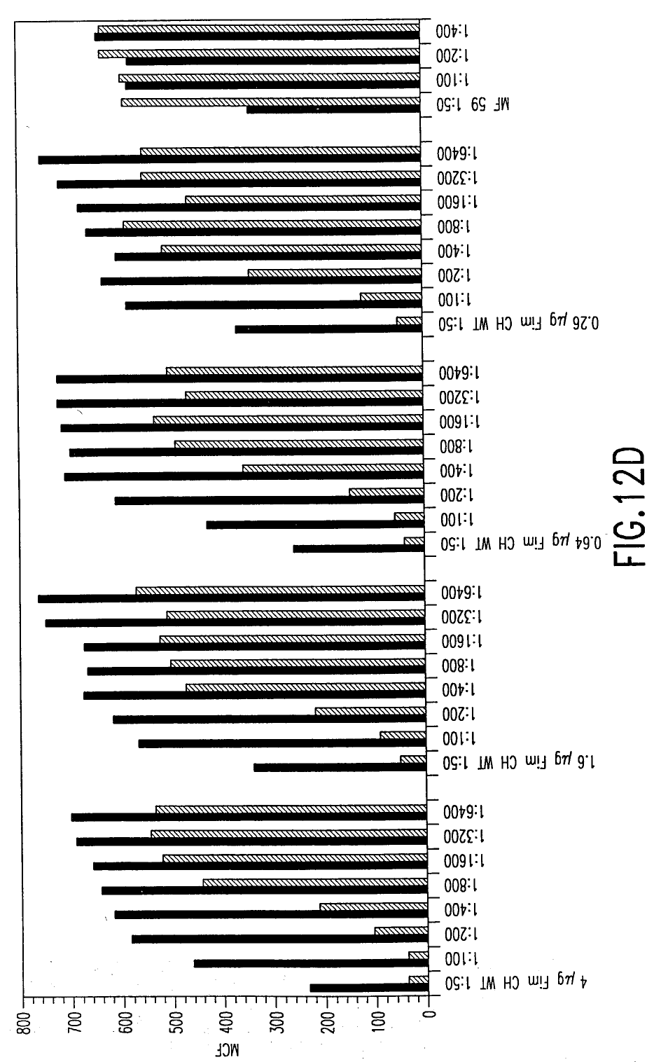

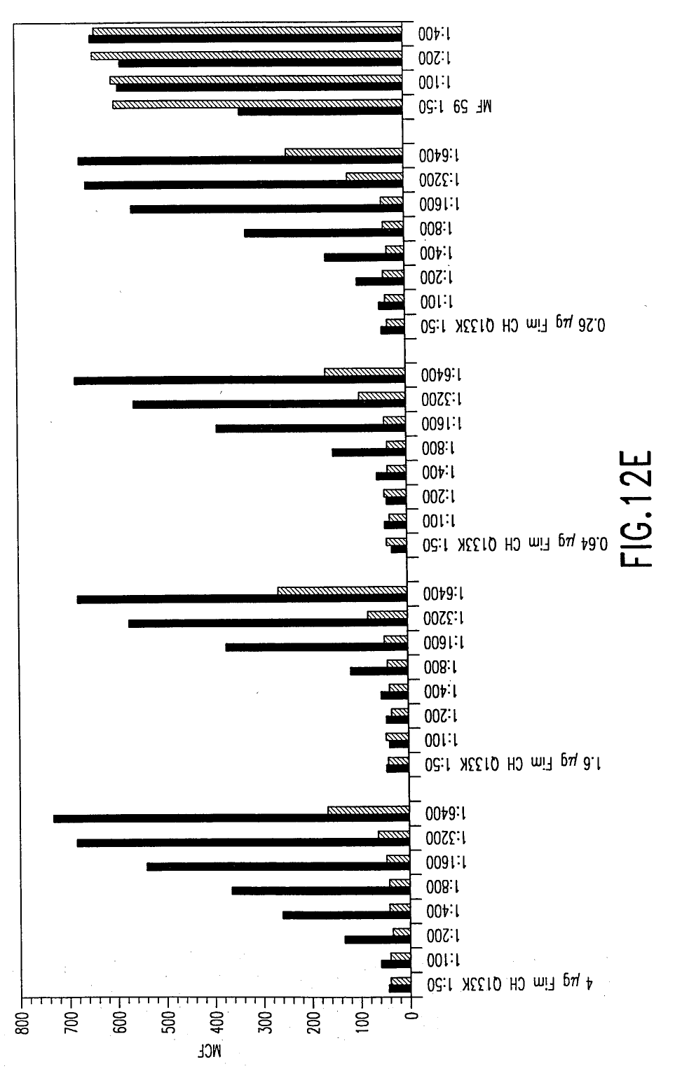

The above functional inhibitory assay was performed using the mutant FimH proteins of the invention. Inhibitory assays were run with the 8 week antisera (4 weeks post boost) from mice vaccinated with FimCH N46D and FimCH D140E and the antisera showed comparable inhibitory titers to the anti-FimH wild type antisera. (FIG. 12A and 12B).

Although the absolute titers were low, antibodies to FimCH Q133K had a better in vitro functional inhibitory activity when compared to wild type FimH antibodies (FIG. 12C). This trend toward superior inhibitory function continued past the 4 week boost. Antisera from mice receiving the 4.0, 1.6, and 0.64 doses of the FimCH Q133K protein was still 100% inhibitory at a 1:1600 dilution. Antisera from mice receiving the 0.26 dose of the mutant protein was still 75% inhibitory at the 1:1600 dilution. This is contrasted with the endpoint inhibitory titer of 1:400-1:800 dilution seen at the highest dose (4.0 μg) for wild type FimCH protein.

For wild type FimCH and FimCH Q133K, an additional boost at week 18 was given. Inhibitory assays were done with antisera from week 16 and week 20. At week 16 (before the second boost), anti-wild type FimCH antibodies did not inhibit bacteria binding to the bladder cells well (FIG. 12D). This is contrasted with anti-FimCH Q133K antibodies. At higher concentrations of antibodies (i.e. 1:50, 1:100, and 1:200 dilutions), the pre-second boost anti-FimCH Q133K still retain inhibitory ability (FIG. 12E). At 20 weeks (2 weeks post second boost), the anti-wild type FimCH antibody does regain some inhibitory ability but it is not as dramatic as the anti-FimCH Q 133K antibody.

Polyclonal antibodies to WT FimCH can inhibit bacteria binding to uroepithelial cells from diabetic women. Uroepithelial cells were isolated from the urine of diabetic women. FITC-labeled E. coli strain NU14 (expressing WT FimCH) was incubated with polyclonal antibodies to FimC, FimH or FimCH. This decreased bacterial binding to the uroepithelial cells by 65% (data not shown).

6.3.2 In Vivo

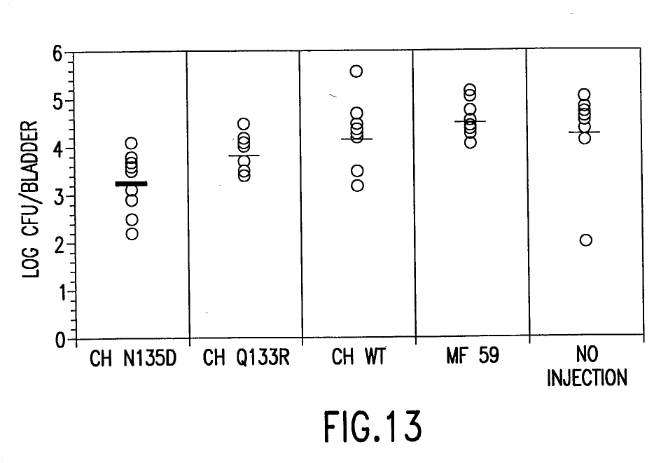

Mice were passively immunized with polyclonal antibodies generated with either WT FimCH or mutant protein (FimCH N135D or FimCH Q133R). Mice were administered 1 mg of polyclonal antibody 4 hours prior to a large bolus challenge live uropathogenic E. coli. Type 1 piliated E. coli strain (NU14) bacteria were collected, washed and re-suspended in phosphate buffered saline (PBS) and cell concentration adjusted to OD=1.8 (at 600 nm). This bacterial cell suspension was then diluted 1:10 in PBS and used as inoculum. Mice were anaesthetized and then inoculated intraurethrally with 50 ml of E. coli suspension containing about 3×10⁷ CFU (colony forming units). CFU determination was done by plating the bacterial suspension on TCA plates and examining cell viability. Two days post-inoculation, the mice were sacrificed and bladders were removed and collected into 500 ml PBS supplemented with 1% mannose. The number of CFUs per bladder was determined by grinding the bladders with a tissue tearer and then plating the suspension on TSA plates after dilution. The mean number of colony forming units per bladder was determined and data was transformed to log CFU/bladder. A decrease in the number of CFUs indicates that the passive immunization had a protective ability. Polyclonal antibodies to both mutant proteins were more protective than those raised against wild type protein (FIG. 13). The decrease in CFUs per bladder obtained by administration of polyclonal antibodies raised against mutant FimCH was significant when compared to CFUs per bladder obtained when no antibody was administered as indicated by a T-test (see Table 11).

TABLE 11
T-test Results

| antigen polyclonal | | |
|---|---|---|
| antibody raised against | MF 59 alone | no injection |
| FimCH | 0.190 | 0.581 |
| FimCH N135D | 0.00003 | 0.0043 |
| FimCH Q133R | 0.0004 | 0.080 |

### 6.4 Example 4

### Inhibitory Properties of Monoclonal Antibodies

6.4.1 in vitro

Functional inhibitory properties of antibodies were measured by the ability to block binding of type 1 piliated bacteria (E. coli strain NU14) to guinea pig erythrocytes in a hemagglutination assay and by the ability to inhibit E. coli binding to an ELISA plate when tri-mannose was the capture antigen. Fab fragments were also assayed for inhibitory activity.

Hemagglutination Assay

The bacteria were directly labeled with fluorescein isothicyanate (FITC) and incubated with the antibody to be assayed for 30 minutes at 37° C. The bacteria/antibody mixture was then added to the erythrocytes and allowed to incubate. After multiple washes, mean channel fluorescence was used as an indicator of the amount of FITC-labeled bacteria remaining (and thereby is an indication of the strength of the interaction between the FimCH complex on the E. coli and mannose). Lysis II software (Becton Dickinson Immunocytometry Systems) was used for analysis of data.

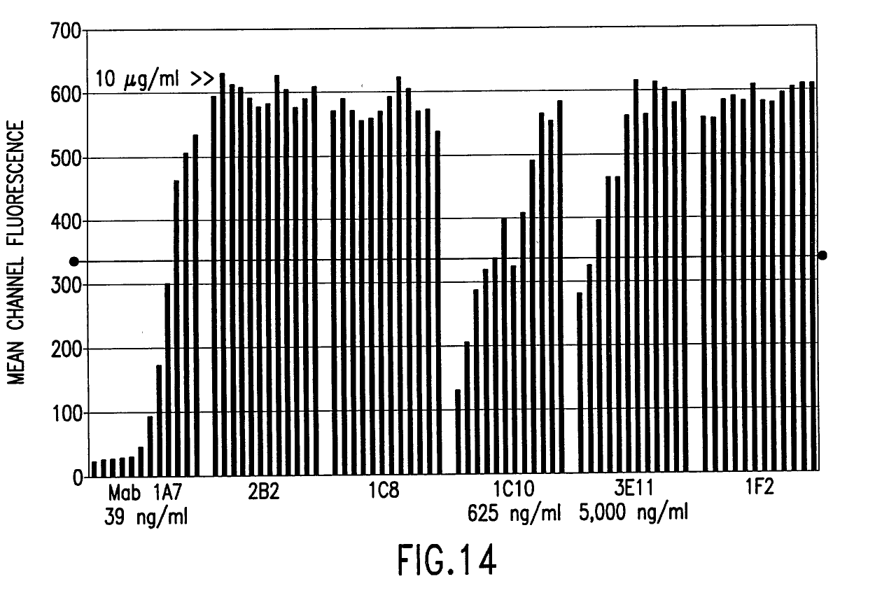

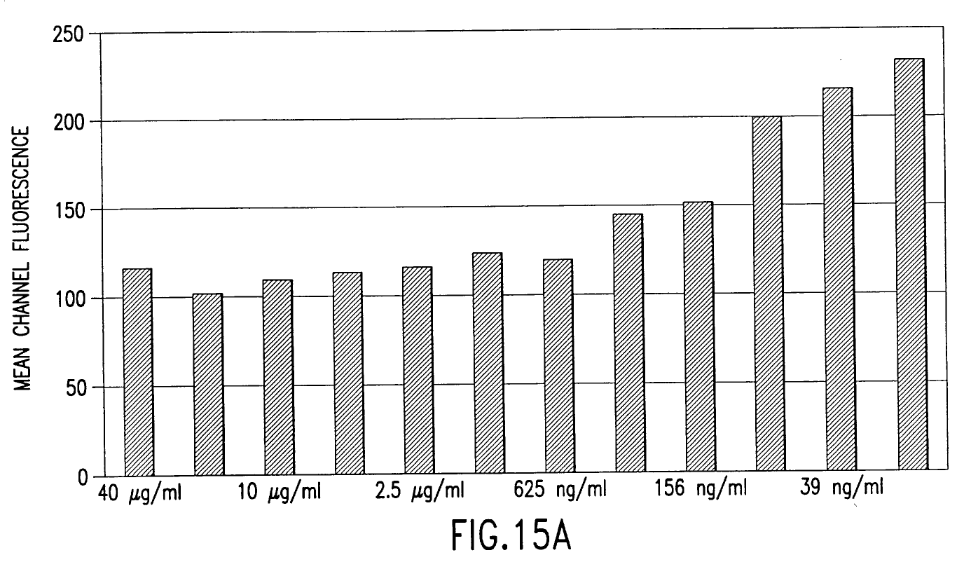

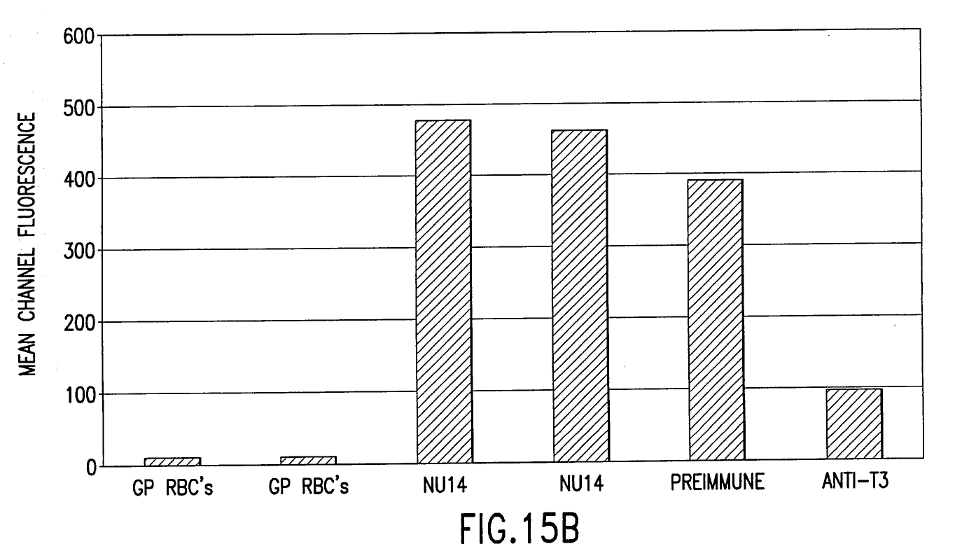

FIG. 14 shows the results from the hemagglutination assay. Increasing dilutions of MAB clone were used in a set of parallel experiments. Preincubation with clone 1A7 inhibited bacteria binding to the erythrocytes very strongly. Clones 1C10 and 3E11 also inhibited bacteria binding when the MABs were supplied in larger quantities. Clones 1F2, 2B2, and 1C8 did not show an inhibitory activity. FIG. 15A shows the results of various concentrations of clone 1A7 used in the hemagglutination assay. FIG. 15B shows various controls that indicate that this inhibitory activity was due to preincubation with MAB clone 1A7. Guinea pig red blood cells alone do not fluoresce. E. coli Nu14 bind to guinea pig red blood cells in the absence of antibody pre-incubation. Pre-incubation of E. coli with pre-immune serum does not inhibit binding to guinea pig red blood cells. As expected, pre-incubation with antibodies raised against T3 (a histidine tagged N-terminal lectin binding domain of FimH from amino acid residues 1-184) does inhibit E. coli binding to guinea pig red blood cells.

ELISA Binding Assay

Immulon 4 plates were coated overnight at 4° C. with 0.1 μg/well of tri-mannose-BSA. On the following day, wells were blocked with 300 ul/well of PBS+1% BSA+0.02% Sodium azide for 1 hour at 37° C. followed by three washes with PBS+0.05% Tween-20 (PBST). E. coli that had been pre-incubated with the antibody to be assayed (for 30 minutes at 37° C.) was added to the tri-mannose coated well. After incubation, the wells were washed extensively. Optical density at 450 nm (OD₄₅₀) was recorded and used as an indicator of the amount of bacteria attached to the tri-mannose.

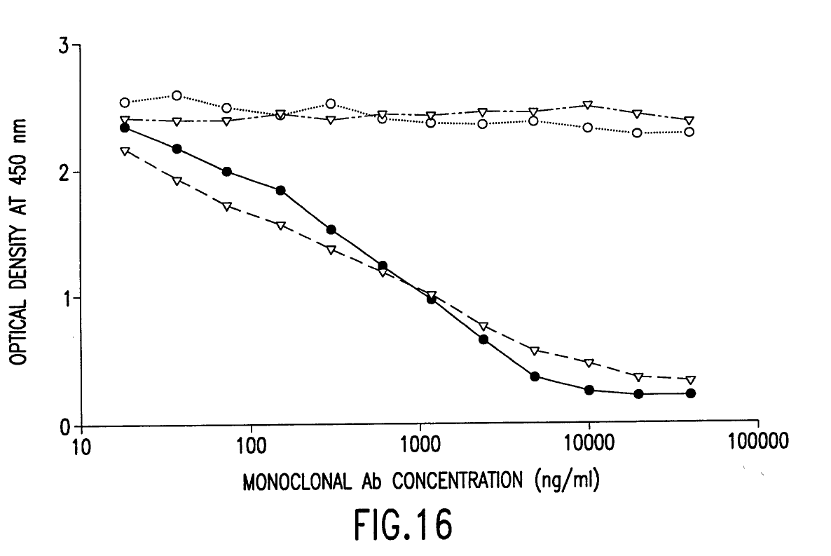

FIG. 16 shows the results from the ELISA assay. Pre-incubation of bacteria with either MAB clone 1A7 or 1C10 did inhibit binding to tri-mannose as evidenced by the decrease in OD₄₅₀ with increasing MAB antibody used. MAB clone 1C8 (which recognizes an epitope on FimC) did not demonstrate any inhibitory effect at any amount of MAB used and thus mimicked the negative control data.

Characterization of Fab Fragments

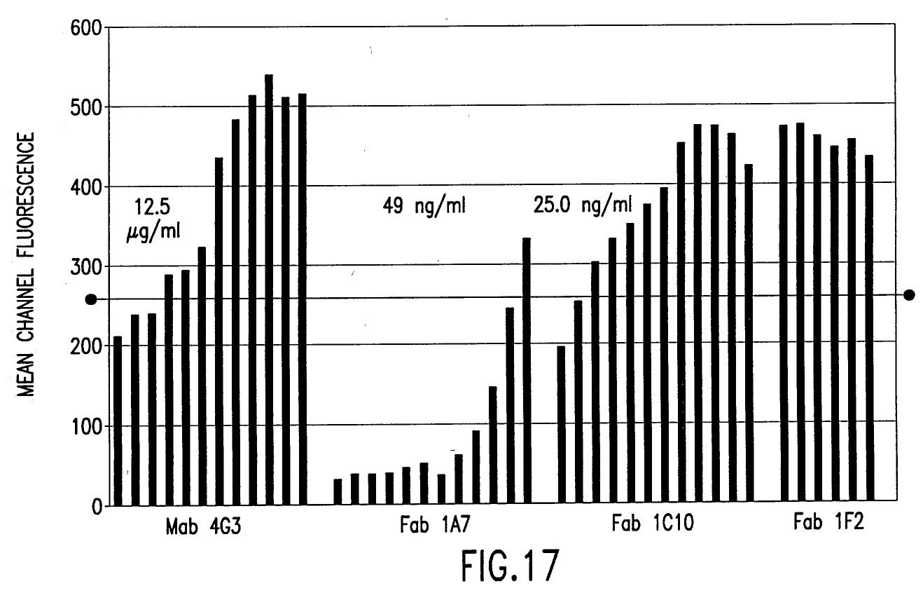

Fab fragments were generated for MAB clones 1A7, 1C10 and 1F2. Fabs were purified before use as potential inhibitors of FimCH-mannose binding in a hemagglutination assay. The assay was done as previously, with results shown in FIG. 17. Fab fragments of clone 1A7 inhibited bacteria binding as well as intact MAB clone 1A7. This suggests that clone 1A7 inhibits FimCH binding through a steric hindrance of binding versus agglutination. However, Fab fragments of clone 1C10 displayed a drastic decrease in inhibitory ability when compared with its intact MAB counterpart. This suggests that agglutinating activity is an important part of clone 1C10 MAB's inhibitors. activity.

6.4.2 In Vivo

Passive immunization protection studies were done with MAB clones 1A7, 1C10, and 1F12. One mg of purified MAB was administered by IP injection to a C3H/HeJ mouse. Four hours after MAB administration, the mouse was challenged inatraurethrally with 8.2×10⁷ CFU of uropathogenic E. coli NU14. After 48 hours, the animal was sacrificed and the bladder was harvested to determine the resulting CFU per bladder.

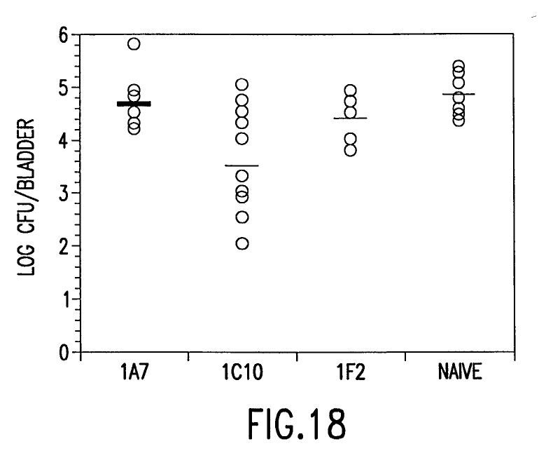

FIG. 18 shows the results of the passive immunization experiment. MAB clone 1C10 provided significant protection (1.4 log reduction in CFU) against E. coli infection. However, neither MAB clone 1A7 or 1F2 showed the ability to protect against the large bolus challenge. The decrease in CFUs per bladder obtained by 1C10 administration was significant when compared to CFUs per bladder obtained when no MAB was administered as indicated by a T-test (see Table 12).

TABLE 12
T-test Results

| MAB clone | no injection |
|---|---|
| 1A7 | 0.271 |
| 1C10 | 0.002 |
| 1F2 | 0.024 |

### 6.5 Example 5

### Use of Mutant Proteins as Vaccines

The purpose of these studies is to examine the efficacy of FimCH mutant to induce a protective immune response in primates.

6.5.1 Monkey Vaccination

A recombinant FimC and a mutant FimH complex is purified to over 99% purity from the periplasm of E. coli K12 strain 600 as described in Jones et al. (1993, Proc. Natl. Acad. Sci. USA 90:8397-401).

Bacteria is cultivated in LB agar. Expression of type 1 pili is induced by two 48 hour passages in static brain-heart infusion broth (Difco Labs, Detroit) culture at 37° C. Before infection, expression of type 1 pili is quantitated by titration of bacterial suspension and mixing of equal volumes of 3% yeast cells and bacteria in microtiter cells to assay agglutination titers (titers equal to or over 30-60 indicate type 1 pili expression). After bacterial challenge in the monkeys, urine samples from days 2, 4, 7 and 12 after challenge are counted by streaking 100 L of serial 10 step dilution onto cystine-lactose-electrolyte deficient agar plates by means of sterile plastic disposable loops. After incubation overnight at 37° C., E. coli colonies are counted to establish the number of CFU/ml in the urine. A urine specimen is considered positive when it contains at least 100 CFU/ml. To establish that inoculating strain was recovered in urine, urinary bacteria are biochemically analyzed on prepared microplates for rapid typing of coli form bacteria using PhenePlate systems.

The surfactant stabilized emulsion adjuvant MF59 is used to emulsify the mutant FimCH complex and for adjuvant administration. Cynomolgus monkeys receive either 100 μg of mutant FimCH in MF59 adjuvant at a 1:1 ratio, or MF59 plus diluent at weeks 0, 4, and 48. Each 1 ml injection is administered intramuscularly in the thigh (legs are alternated for each injection). Serum samples are collected once a month after vaccination for assessment of immune responses.

Vaginal wash and serum samples are also collected before and after the last boost (weeks 47 and 50). The vaginal wash samples are diluted 1:2 in 0.5% bovine serum albumin, 0.5% milk and 0.2% azide before analysis. Antibody levels are recorded as actual OD at 405 nm (values <2× background were considered negative).

In addition, functional assays are performed with the serum and vaginal washes to demonstrate the efficacy of the vaccine to induce an anti-FimH immunoglobulin response.

With respect to the serum samples, type 1 piliated NU14 E. coli are directly labeled with fluorescein isothiocyanate and incubated with 10⁶ J82 bladder cells at a ratio of 250 bacteria/cell in the presence of preimmune or immunized serum and in cubated for 30 minutes at 37° C. After multiple washes, samples are assayed by flow cytometry, and percent inhibition is determined relative to preimmune samples from each monkey.

Vaginal washes are also tested to determine if the titer of antibodies in the washes of vaccinated subjects are sufficient to inhibit E. coli binding to trimannose. Briefly, 2.5 μg/ml of trimannose-bovine serum albumin is coated on Immulon-4 plates (Dynex Technologies, Chantilly, Va.). Type 1 piliated NU14 bacteria (8.0×10⁷ CFU/ml) is added to each well, incubated at 37° C. for one hour, washed extensively and bound bacteria are detected with 1:400 dilution of anti-E. coli horseradish peroxidase conjugated antibody (BioDesign, Kennebunk, Me.). Percent inhibition is assessed as a ratio, where % inhibition=[(full signal values—sample value)/full signal value]×100.

All test monkeys are infected 18 days after the final immunization with E. coli. Bladder infection is induced by inoculation of bacterial suspension (1 ml, 10⁸ CFU/ml) via urethral catheter. Urine samples are obtained on days 2, 4, 7, 12 and 14 after challenge to determine the number of bacteria per milliliter of urine, as a measure of injection. Urine samples are also tested for leukocytes as an indicator of inflammation.

Normal flora is also tested to determine whether systemic vaccination with the mutant FimCH adhesin polypeptide affects the normal intestinal flora. E. coli recovered from fecal suspensions from each monkey is tested in the PhP assay. All monkeys in both vaccine groups showed normal coliform bacterial growth.

6.5.2 Human Vaccination

Recombinant highly purified mutant FimCH is formulated in the squalene-based adjuvant MF59C.1 to examine safety and immunogenicity in a randomized, controlled, double blind Phase I clinical trial in healthy adult women who are seronegative for anti-FimH antibodies.

Methods

The soluble 52 kDa recombinant protein complex of FimC and mutant FimH, FimCH, is recovered from lysed bacteria using a three step chromatographic, process. The bulk product is sterile filtered and vialed in a citrate buffer. Shortly before injection into a subject, the FimCH composition is mixed with a squalene-based emulsion adjuvant known as MF59C.1 (Chiron Corp., Calif.).

In vitro binding to human tissues, purified receptors or receptor homologues is often used to elucidate the roles in virulence of many different adhesins, including pilus-associated adhesins. Similarly, assaying for the ability of such antibodies to block attachment of bacteria to cells or specific receptors can assess the functionality of antibodies to adhesins. This allows for rapid in vitro assessment of serological cross-reactivity between antibodies raised to a single adhesin, such as FimCH purified from one strain of E. coli, against a wide range of E. coli clinical isolates expressing highly homologous, yet phenotypically distinct FimH adhesins.

The ability of the anti-FimH adhesin antibodies to block bacterial binding to bladder epithelial cells is investigated in vitro using a flow cytometric method originally developed for evaluating Rickettsia-cell attachment (Li and Walker, 1992, Infect Immun. 60:2030-5, which is incorporated herein in its entirety).

The bacterial binding inhibition assay is run as follows. Type 1-piliated E. coli (cystitis, pyelonephritis, gut etc.) isolates are directly labeled with FITC and incubated with 2×10⁶ J82 bladder cells, at a ratio of 250 bacteria/cell, in the presence of pre-immune or hyper-immune serun (murine, rabbit, primate or human antisera) and allowed to mix with the bacteria for 30 minutes at 37° C. Antisera are added at dilutions typically ranging from 1:50 to 1:6400 (two-fold serial dilutions). After multiple washes, samples are assayed by flow cytometry in a FACStar PLUS (Becton Dickinson) according to previously published methods (Langermann et al., 1997, Science, 276:607-11). Mean channel fluorescence is used as an indicator of FITC-labeled bacteria bound to J82 bladder cells.

Endpoint inhibitory titers are defined as the titer, after serial two fold dilutions, at which the MCF value (representing bacteria bound to cells) is less than or equal to 50% of the MCF value for the control samples (where control is bacteria incubated with pre-immune serum). To confirm binding and inhibition, J82 bladder cells can be sorted From the flow cytometric adherence assay described and analyzed by fluorescent microscopy and the number of fluorescent bacteria attached to 40 bladder cells visually quantitated.

This assay can be run with vaginal wash samples as long as the samples are collected by straight lavage (“PBS washes”). For vaginal wash samples, inhibitory titer ratios are measured for all samples at a 1:2 dilution. Inhibition cannot be run with vaginal antibody samples collected by the cel-wec method, as this method relies upon a detergent-based extraction buffer which interferes with the binding assay.

Functional inhibitory antibodies to FimCH are also evaluated in an assay called the E. coli trimannose-binding assay. Briefly, Immulon-4 plates (Dynex Technologies, Inc., Chantilly, Va.) are coated with 2.5 μg/ml (100 ml/well) of tri-mannose-BSA (V-Labs, Covington, La.). Type 1-piliated NU14 (8.0×10⁷ CFU/ml) are added to each well, incubated at 37° C. for 1 hour and after extensive washing, bound bacteria are detected with a 1:400 dilution of an anti-E. coli-HRP conjugated antibody (BioDesign, Kennebunk, Me.). OD₄₅₀ readings of these samples establish the full signal values (FSV) for binding to trimannose (approximately 2.0). Additional samples are run in the presence of 1:50 dilutions of serum to assess inhibition, where percent inhibition equals the FSV—the sample value/FSV×100. All samples are run in triplicate.

Antibody sampling of vaginal secretions from primates was performed with a sterile cotton swab. The swab was then suspended in 1 ml of PBS, yielding the solution to test for antibodies. The samples were centrifuged at 2,000 × g for 10 minutes it 4° C. The supernatant was treated with Nonidet P-40, aliquoted and stored at −70° C. Antibody sampling of cervical secretions from humans was performed using an absorbent sponge called a Cel-Wec. Cervical secretions (Immunoglobulin) were eluted from sponges “Weck-Cel Spears” with elution buffer: 1 × PBS, 0.5% IGEPAL® (nonionic detergent), Protease inhibitors (1 mg/ml Aprotinin, 1 mM Leupeptin, Bestatin). Antibody sampling of urine samples was done on straight, undiluted urine samples from “clean catch” specimens.

Quantitation of Human IgG in Serum/Urine/Cervical Secretion Samples ELISA Procedure

96 well ELISA plates are coated with capture antibody:

mouse anti human IgG (1 μg/ml CO3 buffer)

Standard*: Human IgG whole molecule (1000 ng-977 pg/ml)

Samples: Human urine or cervical secretions in PBS (diluted two fold 1:2 to 1:64)

Secondary: Biotin labeled goat F(ab′2) anti-human IgG

Tertiary: streptavidin Horse Radish Peroxidase

Substrate: TMB

Plates are read at 450nm and quantity determined by SOFTmax software * to generate a standard curve this is run along with the urine, cervical secretion samples

In order to determine IgG quantity, each urine and cervical secretion sample is run in duplicate at six different dilutions (for all individuals tested). The quantity for each dilution is automatically calculated by SOFTmax using a 4 parameter standard curve (range 1000 ng-977 pg/ml). Only the quantities derived from OD values that fall within the linear range of the standard curve are used to determine the amount of IgG in a serum sample. These quantities are averaged to determine amount of IgG in a sample.

Clinical Procedures

Four cohorts of 12 subjects are randomized at a ratio of 3:1 ( i.e., four groups where nine subjects receive the vaccine and 3 subjects receive the adjuvant alone) and, in a sequential fashion, given intramuscular doses of vaccine or control. Mutant FimCH is prepared for injection into a subject immediately prior to the injection, i.e., mixed with diluent and adjuvant. Doses of either 1, 5, 25 or 123 μg of mutant FimCH in 0.5 ml of MF59C.1, or the control (MF59C.1 alone) are injected slowly, i.e., 20 to 30 seconds, into the deltoid muscle of the upper arm of the subjects at day 0, followed by a booster dose at about 28 days followed by a second booster dose at about 180 days.

To test if the mutant FimCH vaccine is immunogenic in the human subjects, evidence of a clear dose response is looked for. Serum, urine, and vaginal secretions of vaccine recipients is used in Western blot and ELISA assays to determine levels of anti-mutant FimH antibodies. Also, immune serum from vaccine recipients is assayed for inhibitory activity by addition to uropathogenic E. coli before exposure to J82 human uroepithelial cell line (bladder cells) in vitro. Inhibition of binding of E. coil to J82 cells indicates the presence of inhibitory antibodies.

### 6.6 Example 6

### Preparation of Co-Crystals of FimCH and α-D-Mannopyranoside

The subsections below describe the production of the FimCH complex and the preparation and characterization of diffraction quality co-crystals of FimCH with α-D-mannopyranoside.

6.6.1 Production and Purification of FimCH

Plasmid pHACW18 was constructed by cloning fimH into the EcoR I and BamH I sites of pUC18 (Norrander et al., 1983 Gene 26:101-6). Briefly, the fimH gene was amplified from pHJ20 (Jones et al., 1995 Proc Natl Acad Sci U S A. 92:2081-5) by polymerase chain reaction (PCR) using Vent Polymerase (New England Biolabs). The resulting fimH gene was confirmed by sequencing. Plasmid pHJ9205 contained the fimC open reading frame driven by the inducible arabinose promoter and was used for the co-expression of FimH proteins. The plasmid pUT2002 having a fimH deleted type 1 operon driven by the natural promoter was described previously (Minion et al., 1989, J Bacteriol 165:1033-6).

The E. coli strain C600 (Sambrook et al., Molecular Cloning: A Laboratory Manual, 2^{nd} ed., Cold Spring Harbor Laboratory Press, New York (1989)) was used in this study. All bacteria used were grown in Luria Broth (LB) with appropriate antibiotics. Periplasmic extracts were isolated as described (Slonim et al., 1992, EMBO J. 11:4747-56). Bacterial strain C600/pHJ9205 transformed with FimH expression constructs was used to produce large quantities of FimH proteins. These transformants were grown in LB in the presence of 0.1% arabinose and 0.1 mM IPTG to induce FimC and FimH expression, respectively. The protocol for the purification of FimCH complexes from bacterial periplasm has been described previously and was followed in this study (Barnhart et al., 2000, Proc. Natl. Acad. Sci. USA 97:7670-2), which is hereby incorporated by reference in its entirety. Purified FimCH complexes were dialyzed into 20 mM MES, pH 6.8 and stored at 4° C.

6.6.2 Preparation of FimCH-α-D-Mannopyranoside Co-Crystals

The FimCH complex was co-crystallized with α-D-mannopyranoside by vapor diffusion in 4 ml hanging drops. 2 ml of FimCH at OD 5.9 (4.7 mg/ml) in 20 mM MES pH 6.5 and 7 mM α-D-mannose was mixed with 2 ml of 1.0 M (NH₄)₂SO₄ and 100 mM TRIS-HCl pH 8.2 and equilibrated against the latter solution. After 1 week the drops were streak seeded from drops containing small crystalline FimCH. Single bipyramidyl crystals about 0.4 mm large in each dimension were fully grown after 2 weeks. The crystals were frozen in after sequentially washing in 1.2 M (NH₄)₂SO₄, 100 mM Tris pH 8.2 complemented up to a final 25% glycerol in steps of 5% glycerol.

6.6.3 Analysis and Characterization of FimCH-α-D-Mannopyranoside Co-Crystals

Diffraction Data Collection

Diffraction data sets were collected at beamline 19BM at Advanced Photon Source, Argonne, USA. Processing of the data was performed with an HKL2000 (Otwinowski & Minor,1997, Methods in Enzymology 276:307-326). The crystals were frozen after sequentially soaking in 5% up to a final 25% glycerol in 1.2 M (NH₄)₂SO₄ and 100 mM Tris pH 8.2. The space group was C2 with strong pseudotetragonal features. Unit cell dimensions were a=138.077, b=138.130, c=215.352, b=90.005 for FimCH mannose.

Structure Determination

Rigid body refinement was performed using the FimCH structure (PDB entry code 1QUN) as the model. The refinement was started using a high temperature (3500 K) slowcool stage to remove model bias. Subsequent positional and individual B-factor refinements were performed without s cut-off, using CNS (Brunger et al., 1998, Acta Crystallogr D Biol Crystallogr. 54:905-21). At this stage, electron densities were inspected and four of the eight molecules in the asymmetric unit were found to have good electron density, in contrast with their four non-crystallographically related partners that had a significant part of the pilin domain of the adhesin and the chaperon disordered. The electron density of the receptor binding domain of the adhesin of all eight of the FimCH molecules was clearly defined and showed a mannoside in the carbohydrate binding pocket. Refinement and model building led to final R_{free} and R factors of 0.279 and 0.239 (50-2.8 Å) for FimCH mannose.

Structure Analyses

Table 13 summarizes the X-ray crystallography refinement parameters of the structure of the crystalline FimCH-α-D-mannopyranoside co-complex of the invention.

TABLE 13
Data Collection and Refinement Summary

| space group | | C2 |
|---|---|---|
| unit cell | a (Å) | 138.077 |
| | b (Å) | 138.130 |
| | c (Å) | 215.352 |
| | b (°) | 90.005 |

| | |
|---|---|
| Molecules per asymmetric unit | 8 |
| Resolution | 50.0-2.8 |
| number of observed reflections | 370.427 |
| number of unique reflections | 99.138 |
| highest resolution shell | 2.9-2.8 |
| R-merge (%) | 6.9 (47.8) |
| completeness (%) | 99.8 (99.9) |
| <I/s(I)> | 13 (2.7) |
| reflections with I > 2 | 83.8 (52.4) |
| Number of protein atoms | 29.168 |
| Number of water molecules | 636 |
| sigma cut-off used in refinement | None |
| crystallographic R-factor | 0.239 (0.35) |
| R_{free} | 0.279 (0.42) |
| r.m.s. bond lengths (Å) | 0.007 |
| r.m.s. bond angles (deg.) | 1.4 |

Table 14 provides the atomic structure coordinates of the crystalline FimCHα-D-mannopyranoside co-complex in Protein Database Format. The amino acid residue numbers coincide with those used in FIG. 2.

Structures coordinates for the crystalline FimCH-α-D-mammopyranoside co-complex according to Table 13 may be modified by mathematical manipulation. Such manipulations include, but are not limited to, crystallographic permutations of the raw structure coordinates, fractionalization of the raw structure coordinates, integer additions or subtractions to sets of the raw structure coordinates, inversion of the raw structure coordinates and any combination of the above.

6.6.4 Mutant FimCH-α-D-Mannopyranoside Co-Crystals

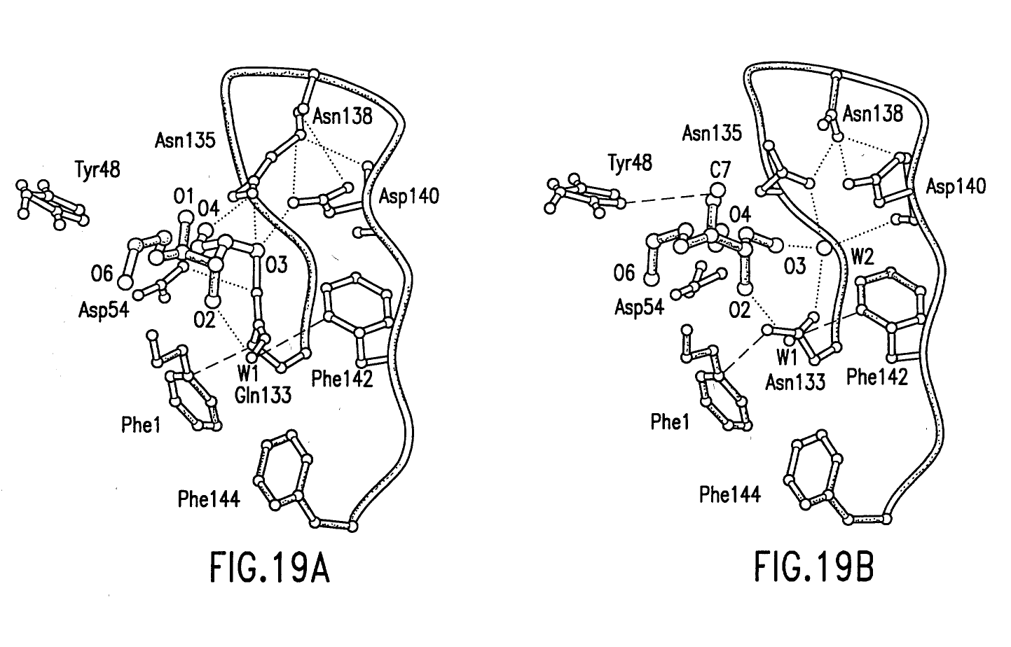

The structure of the FimCH complex containing the Q133N mutation, derived from crystals grown in the presence of methyl-α-D-mannopyranoside, shows binding of the receptor (FIG. 19B). The electron density is strongest at positions C4, C5 and C6 of the sugar. The Clinked methyl group on the anomeric O1 of mannose points outwards away from the pocket and makes a hydrophobic contact with Tyr48 (at 3.7 Å). Asn133 does not link to O3 of the mannose. Interestingly, the Q133N mutation not only affects the interactions originally made by Gln133, but the mannose also loses interaction with Asp140 and Asn135 (FIG. 19). The mannose has shifted 0.7 Å from its position in the wild type. A shift in the protein backbone at Asp140 of about 0.7 Å together with changes in the side chain conformations of the Asn133, Asn135, Asn138 and Asp140 enables these residues to take part in a very different hydrogen bonding network (FIG. 19B) than was present in the wild type FimCH-mannose structure (FIG. 19A). This new hydrogen bonding network includes a new water molecule, W2, that interacts directly with O3. In contrast, the O2 ligand residues remained conserved including W1. W1 interacts both with O2 and the amide group of amino acid 133, as in the wild type complex. The hydrophobic part of the Gln133 side chain makes close van der Waals contacts with the Phel aromatic ring (FIG. 19A). The shorter Asn133 side chain compensates for the lack of the penultimate carbon Cγ of Gln133 by establishing an amino-aromatic stacking interaction: Asn133 points its amide nitrogen atom towards the Phe 1 ring (FIG. 19B). Phe1 further stacks with Phe 144. These stacking interactions in the strands holding the loop between Gln133 and Phe142 support the bottom part of the binding site formed by Asn46, Asp47 and Asp54. These results therefore explain how mutating a side chain can dramatically affect the structure of the mannose binding pocket.

TABLE 15
Data Collection and Refinement Summary

| space group | | C2 |
|---|---|---|
| unit cell | a (Å) | 138.349 |
| | b (Å) | 138.334 |
| | c (Å) | 213.212 |
| | b (°) | 89.983 |

| | |
|---|---|
| Molecules per asymmetric unit | 8 |
| Resolution | 45-3.0 |
| number of observed reflections | 197,848 |
| number of unique reflections | 72,289 |
| highest resolution shell | 3.11-3.0 |
| R-merge (%) | 8.7 (51.0) |
| completeness (%) | 87.1 (65.9) |
| <I/s(I)> | 10.6 |
| reflections with I > 2 | 82.3 (60.8) |
| Number of protein atoms | 29,160 |
| Number of water molecules | 377 |
| sigma cut-off used in refinement | None |
| crystallographic R-factor | 0.236 (0.36) |
| R_{free} | 0.280 (0.39) |
| r.m.s. bond lengths (Å) | 0.007 |
| r.m.s. bond angles (deg.) | 1.3 |

Table 16 provides the atomic structure coordinates of the crystalline FimCH Q133N-α-D-mannopyranoside co-complex in Protein Database Format. The amino acid residue numbers coincide with those used in FIG. 19.

Structures coordinates for the crystalline FimCH-α-D-mammopyranoside co-complex according to Table 15 may be modified by mathematical manipulation. Such manipulations include, but are not limited to, crystallographic permutations of the raw structure coordinates, fractionalization of the raw structure coordinates, integer additions or subtractions to sets of the raw structure coordinates, inversion of the raw structure coordinates and any combination of the above.

Tables 14 and 16 are submitted on compact disc pursuant to 37 C.F.R. § 1.52 (e)(1)(iii) and are hereby incorporated by reference.

Equivalents

Those skilled in the art will recognize, or be able to ascertain using no more than routine experimentation, many equivalents to the specific embodiments; of the invention described herein. Such equivalents are intended to be encompassed by the following claims, without undue experimentation, to make such substitutions as will optimally serve their purposes in using the methods and procedures disclosed herein.

We claim:

1. A co-crystal comprising FimC, FimH and mannopyranoside in crystalline form.

2. The co-crystal of claim 1 in which the FimC or FimH is a mutant.

3. The co-crystal of claim 2 in which the mutant is a conservative mutant.

4. The co-crystal of claim 2 in which the FimH is FimH Q133N

5. The co-crystal of claim 2 in which the FimH comprises amino acids 1 to 158 of SEQ ID NO: 4.

6. The co-crystal of claim 1, which is diffraction quality.

7. The co-crystal of claim 1, which is a native crystal.

8. The co-crystal of claim 1, which is a heavy-atom derivative crystal.

9. The co-crystal of claim 1, which is characterized by a unit cell of a=138.077±0.2 Å, b=138.130±0.2 Å, c=215.352±0.2 Å, α=90, β=90.005, and γ=90.

10. The co-crystal of claim 1, which is produced by a method comprising the steps of:
(a) mixing a volume of a solution comprising FimC, FimH and mannopyranoside with a volume of a reservoir solution comprising a precipitant; and
(b) incubating the mixture obtained in step (a) over the reservoir solution in a closed container, under conditions suitable for crystallization until the crystal forms.

11. The co-crystal of claim 10, wherein the precipitant is present in a concentration between 0.6 M and 1.2 M.

12. The co-crystal of claim 10 wherein the precipitant is ammonium sulfate.

13. The co-crystal of claim 10, wherein the solution further comprises between 50 mM and 100 mM Tris HCl.

14. The co-crystal of claim 10, wherein the solution comprises between 0.5 mM and 30 mM mannopyranoside.

15. The co-crystal of claim 10, wherein the solution has a pH of between 7.8 and 8.6.

16. A method of making the crystal of claim 1, comprising:
(a) mixing a volume of a solution comprising the FimC, FimH and mannopyranoside with a volume of a reservoir solution comprising a precipitant; and
(b) incubating the mixture obtained in step (a) over the reservoir solution in a closed container, under conditions suitable for crystallization until the crystal forms.

17. The method of claim 16, wherein the precipitant is present in a concentration between 0.6 M and 1.2 M.

18. The method of claim 16, wherein the precipitant is ammonium sulfate.

19. The method of claim 16, wherein the solution further comprises between 50 mM and 100 mM Tris HCl.

20. The method of claim 16, wherein the solution comprises between 0.5 mM and 30 mM mannopyranoside.

21. The method of claim 16, wherein the solution has a pH of between 7.8 and 8.6.

22. A machine-readable medium embedded with information that corresponds to a three-dimensional structural representation of a co-crystal comprising FimC, FimH, or a fragment or portion thereof, and a mannose sugar in crystalline form.

23. The machine readable medium of claim 22, in which the crystal is diffraction quality.

24. The machine readable medium of claim 22, in which the crystal is a native crystal.

25. The machine readable medium of claim 22, in which the crystal is a heavy-atom derivative crystal.

26. The machine readable medium of claim 22, in which the FimC or FimH is a mutant.

27. The machine readable medium of claim 26, in which the mutant is a selenomethionine or selenocysteine mutant.

28. The machine readable medium of claim 27, in which the mutant is a conservative mutant.

29. A machine-readable medium embedded with the atomic structure coordinates of FIG. 2, or a subset thereof.

30. A method of identifying a FimC or FimH binding compound, comprising the step of using a three-dimensional structural representation of complex comprising FimC, FimH and mannopyranoside, or a fragment thereof, to computationally screen a candidate compound for an ability to bind FimC or FimH.

31. A method of identifying a FimC or FimE binding compound, comprising the step of using a three-dimensional structural representation of complex comprising FimC, FimH and mannopyranoside, or a fragment thereof, to computationally design a synthesizable candidate compound that binds FimC or FimH.

32. A machine-readable medium embedded with the atomic structure of Table 14 or Table 16, or a subset thereof.

33. A co-crystal comprising FimC, FimH ,and a saccharide.


SEQUENCE LISTING (50 sequences)

SEQ ID NO: 1 - DNA, 726 bp, E. coli
  CDS (1)...(723)
atgagtaataaaaacgtcaatgtaaggaaatcgcaggaaataacattctgcttgctggcaggtatcctgatgttcatggcaatgatggttgccggacgcgctgaagcgggagtggccttaggtgcgactcgcgtaatttatccggcagggcaaaaacaagtgcaacttgccgtgacaaataatgatgaaaatagtacctatttaattcaatcatgggtggaaaatgccgatggtgtaaaggatggtcgttttatcgtgacgcctcctctgtttgcgatgaagggaaaaaaagagaataccttacgtattcttgatgcaacaaataaccaattgccacaggaccgggaaagtttattctggatgaacgttaaagcgattccgtcaatggataaatcaaaattgactgagaatacgctacagctcgcaattatcagccgcattaaactgtactatcgcccggctaaattagcgttgccacccgatcaggccgcagaaaaattaagatttcgtcgtagcgcgaattctctgacgctgattaacccgacaccctattacctgacggtaacagagttgaatgccggaacccgggttcttgaaaatgcattggtgcctccaatgggcgaaagcacggttaaattgccttctgatgcaggaagcaatattacttaccgaacaataaatgattatggcgcacttacccccaaaatgacgggcgtaatggaataa

SEQ ID NO: 2 - protein, 241 aa, E. coli
MSNKNVNVRKSQEITFCLLAGILMFMAMMVAGRAEAGVALGATRVIYPAGQKQVQLAVTNNDENSTYLIQSWVENADGVKDGRFIVTPPLFAMKGKKENTLRILDATNNQLPQDRESLFWMNVKAIPSMDKSKLTENTLQLAIISRIKLYYRPAKLALPPDQAAEKLRFRRSANSLTLINPTPYYLTVTELNAGTRVLENALVPPMGESTVKLPSDAGSNITYRTINDYGALTPKMTGVME

SEQ ID NO: 3 - DNA, 903 bp, E. coli
  CDS (1)...(900)
atgaaacgagttattaccctgtttgctgtactgctgatgggctggtcggtaaatgcctggtcattcgcctgtaaaaccgccaatggtaccgctatccctattggcggtggcagcgccaatgtttatgtaaaccttgcgcccgtcgtgaatgtggggcaaaacctggtcgtggatctttcgacgcaaatcttttgccataacgattatccggaaaccattacagactatgtcacactgcaacgaggctcggcttatggcggcgtgttatctaatttttccgggaccgtaaaatatagtggcagtagctatccatttcctaccaccagcgaaacgccgcgcgttgtttataattcgagaacggataagccgtggccggtggcgctttatttgacgcctgtgagcagtgcgggcggggtggcgattaaagctggctcattaattgccgtgcttattttgcgacagaccaacaactataacagcgatgatttccagtttgtgtggaatatttacgccaataatgatgtggtggtgcctactggcggctgcgatgtttctgctcgtgatgtcaccgttactctgccggactaccctggttcagtgccaattcctcttaccgtttattgtgcgaaaagccaaaacctggggtattacctctccggcacaaccgcagatgcgggcaactcgattttcaccaataccgcgtcgttttcacctgcacagggcgtcggcgtacagttgacgcgcaacggtacgattattccagcgaataacacggtatcgttaggagcagtagggacttcggcggtgagtctgggattaacggcaaattatgcacgtaccggagggcaggtgactgcagggaatgtgcaatcgattattggcgtgacttttgtttatcaataa

SEQ ID NO: 4 - protein, 300 aa, E. coli
MKRVITLFAVLLMGWSVNAWSFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 5 - DNA, 30 bp, Artificial Sequence
  Description of Artificial Sequence Primer
ggggggaattcacccggagggatgattgta

SEQ ID NO: 6 - DNA, 28 bp, Artificial Sequence
  Description of Artificial Sequence Primer
ccagtaggcaccaccacatcattattgg

SEQ ID NO: 7 - DNA, 48 bp, Artificial Sequence
  Description of Artificial Sequence Primer
ctggtcggtaaatgcctggtcagcggcctgtaaaaccgccaatggtac

SEQ ID NO: 8 - DNA, 48 bp, Artificial Sequence
  Description of Artificial Sequence Primer
gtaccattggcggttttacaggccgctgaccaggcatttaccgaccag

SEQ ID NO: 9 - DNA, 43 bp, Artificial Sequence
  Description of Artificial Sequence Primer
gccaatggtaccgctatccctgcgggcggtggcagcgccaatg

SEQ ID NO: 10 - DNA, 43 bp, Artificial Sequence
  Description of Artificial Sequence Primer
cattggcgctgccaccgcccgcagggatagcggtaccattggc

SEQ ID NO: 11 - DNA, 44 bp, Artificial Sequence
  Description of Artificial Sequence Primer
ccataacgattatccggaaaccgcgacagactatgtcacactgc

SEQ ID NO: 12 - DNA, 44 bp, Artificial Sequence
  Description of Artificial Sequence Primer
gcagtgtgacatagtctgtcgcggtttccggataatcgttatgg

SEQ ID NO: 13 - DNA, 24 bp, Artificial Sequence
  Description of Artificial Sequence Primer
gcaacgaggcgccgcttatggcgg

SEQ ID NO: 14 - DNA, 24 bp, Artificial Sequence
  Description of Artificial Sequence Primer
ccgccataagcggcgcctcgttgc

SEQ ID NO: 15 - DNA, 28 bp, Artificial Sequence
  Description of Artificial Sequence Primer
cttttgccatgctgattatccggaaacc

SEQ ID NO: 16 - DNA, 28 bp, Artificial Sequence
  Description of Artificial Sequence Primer
ggtttccggataatcagcatggcaaaac

SEQ ID NO: 17 - DNA, 28 bp, Artificial Sequence
  Description of Artificial Sequence Primer
cttttgccatgatgattatccggaaacc

SEQ ID NO: 18 - DNA, 28 bp, Artificial Sequence
  Description of Artificial Sequence Primer
ggtttccggataatcatcatggcaaaac

SEQ ID NO: 19 - DNA, 55 bp, Artificial Sequence
  Description of Artificial Sequence Primer
gcaaatcttttgccataacgatgcgccggaaaccattacagactatgtcacactg

SEQ ID NO: 20 - DNA, 55 bp, Artificial Sequence
  Description of Artificial Sequence Primer
cagtgtgacatagtctgtaatggtttccggcgcatcgttatggcaaaagatttgc

SEQ ID NO: 21 - DNA, 24 bp, Artificial Sequence
  Description of Artificial Sequence Primer
accattacagcttatgtcacactg

SEQ ID NO: 22 - DNA, 24 bp, Artificial Sequence
  Description of Artificial Sequence Primer
cagtgtgacataagctgtaatggt

SEQ ID NO: 23 - DNA, 24 bp, Artificial Sequence
  Description of Artificial Sequence Primer
accattacaaactatgtcacactg

SEQ ID NO: 24 - DNA, 24 bp, Artificial Sequence
  Description of Artificial Sequence Primer
cagtgtgacatagtttgtaatggt

SEQ ID NO: 25 - DNA, 24 bp, Artificial Sequence
  Description of Artificial Sequence Primer
cttattttgcgcgctaccaacaac

SEQ ID NO: 26 - DNA, 24 bp, Artificial Sequence
  Description of Artificial Sequence Primer
gttgttggtagcgcgcaaaataag

SEQ ID NO: 27 - DNA, 24 bp, Artificial Sequence
  Description of Artificial Sequence Primer
cttattttgcgaaataccaacaac

SEQ ID NO: 28 - DNA, 24 bp, Artificial Sequence
  Description of Artificial Sequence Primer
gttgttggtatttcgcaaaataag

SEQ ID NO: 29 - DNA, 24 bp, Artificial Sequence
  Description of Artificial Sequence Primer
cttattttgcggaagaccaacaac

SEQ ID NO: 30 - DNA, 24 bp, Artificial Sequence
  Description of Artificial Sequence Primer
gttgttggtcttccgcaaaataag

SEQ ID NO: 31 - DNA, 43 bp, Artificial Sequence
  Description of Artificial Sequence Primer
gccgtgcttattttgcgagaaaccaacaactataacagcgatg

SEQ ID NO: 32 - DNA, 43 bp, Artificial Sequence
  Description of Artificial Sequence Primer
catcgctgttatagttgttggtttctcgcaaaataagcacggc

SEQ ID NO: 33 - DNA, 43 bp, Artificial Sequence
  Description of Artificial Sequence Primer
gccgtgcttattttgcgacgcaccaacaactataacagcgatg

SEQ ID NO: 34 - DNA, 43 bp, Artificial Sequence
  Description of Artificial Sequence Primer
catcgctgttatagttgttggtgcgtcgcaaaataagcacggc

SEQ ID NO: 35 - DNA, 43 bp, Artificial Sequence
  Description of Artificial Sequence Primer
gccgtgcttattttgcgacataccaacaactataacagcgatg

SEQ ID NO: 36 - DNA, 43 bp, Artificial Sequence
  Description of Artificial Sequence Primer
catcgctgttatagttgttggtatgtcgcaaaataagcacggc

SEQ ID NO: 37 - DNA, 25 bp, Artificial Sequence
  Description of Artificial Sequence Primer
gcgacagacggccaactataacagc

SEQ ID NO: 38 - DNA, 25 bp, Artificial Sequence
  Description of Artificial Sequence Primer
gctgttatagttggccgtctgtcgc

SEQ ID NO: 39 - DNA, 25 bp, Artificial Sequence
  Description of Artificial Sequence Primer
gcgacagaccgataactataacagc

SEQ ID NO: 40 - DNA, 25 bp, Artificial Sequence
  Description of Artificial Sequence Primer
gctgttatagttstcggtctgtcgc

SEQ ID NO: 41 - DNA, 43 bp, Artificial Sequence
  Description of Artificial Sequence Primer
gcgacagaccaacaacgcgaacagcgatgatttccagtttgtg

SEQ ID NO: 42 - DNA, 43 bp, Artificial Sequence
  Description of Artificial Sequence Primer
cacaaactggaaatcatcgctgttcgcgttgttggtctgtcgc

SEQ ID NO: 43 - DNA, 22 bp, Artificial Sequence
  Description of Artificial Sequence Primer
ctataacagtgcagatttccag

SEQ ID NO: 44 - DNA, 22 bp, Artificial Sequence
  Description of Artificial Sequence Primer
ctggaaatctgcactgttatag

SEQ ID NO: 45 - DNA, 22 bp, Artificial Sequence
  Description of Artificial Sequence Primer
ctataacagcaatgatttccag

SEQ ID NO: 46 - DNA, 22 bp, Artificial Sequence
  Description of Artificial Sequence Primer
ctggaaatcattgctgttatag

SEQ ID NO: 47 - DNA, 22 bp, Artificial Sequence
  Description of Artificial Sequence Primer
ctataacagcgaagacttccag

SEQ ID NO: 48 - DNA, 22 bp, Artificial Sequence
  Description of Artificial Sequence Primer
ctggaagtcttcgctgttatag

SEQ ID NO: 49 - DNA, 43 bp, Artificial Sequence
  Description of Artificial Sequence Primer
gcgacagaccaacaactataacagcgatgatgcgcagtttgtg

SEQ ID NO: 50 - DNA, 43 bp, Artificial Sequence
  Description of Artificial Sequence Primer
cacaaactgcgcatcatcgctgttatagttgttggtctgtcgc